(19) United States
(12) Patent Application Publication
(10) Pub. No.: US 2015/0189884 A1
(43) Pub. Date: Jul. 9, 2015

(54) METHODS OF CONTROLLING INSECTS

(71) Applicant: Syngenta Participations AG, Basel (CH)

(72) Inventors: Myriem El Qacemi, Stein (CH); Jerome Yves Cassayre, Stein (CH)

(73) Assignee: Syngenta Participations AG, Basel (CH)

(21) Appl. No.: 14/422,847

(22) PCT Filed: Aug. 16, 2013
(86) PCT No.: PCT/EP2013/067182

§ 371 Date: Feb. 20, 2015

(30) Foreign Application Priority Data
Aug. 24, 2012 (EP) 12181771.2
Mar. 7, 2013 (EP) 13158160.5

(51) Int. Cl. A01N 43/80; C07D 207/08; C07D 413/10; A01N 43/653; C07D 403/10; C07D 207/20; A01N 43/36; C07D 261/04

(52) U.S. Cl. CPC A01N 43/80; A01N 43/36; C07D 207/08; C07D 261/04; A01N 43/653; C07D 403/10; C07D 207/20; C07D 413/10

(57) ABSTRACT

The present invention provides a method comprising applying to a crop of cotton plants, the locus thereof, or propagation material thereof, a compound of formula (I) wherein -B¹-B²-B³- is —C═N—O—, —C═N—CH₂—, or —N—CH₂—CH₂—; R¹ is trifluoromethyl, difluoromethyl or chlorodifluoromethyl; R² is group X formula (X) X² is C—X⁶ or nitrogen; X¹, X³ and X⁶ are independently hydrogen, halogen or trihalomethyl, wherein at least one of X¹, X³ and X⁶ is not hydrogen; A is selected from A1 to A (A5) B is C—R⁶, CH or nitrogen; 1Y2 and Y3 are independently CH or nitrogen; wherein no more than two of Y¹, Y² and Y³ are nitrogen and wherein Y² and Y³ are not both nitrogen; R is hydrogen, halogen, cyano, nitro, NH₂, C₁-C₄alkyl, C₁-C₄haloalkyl, C₃-C₅ cycloalkyl, C₃-C₅ halocycloalkyl, C₁-C₂ alkoxy, or C₁-C₂ haloalkoxy; provided that when A is A3 or A4 R⁵ is not hydrogen; R⁶ when present together with R⁵ forms a —CH═CH—CH═CH-bridge; R⁷ is C₁-C₄ alkyl;) R⁸ is C₁-C₄ alkyl, C₁-C₄ haloalkyl, C₁-C₄ alkoxy(C₁-C₄)alkyl, C₁-C₄ alkylthio(C₁-C₄)alkyl, C₁-C₄ alkylsulfinyl(C₁-C₄)alkyl, alkylsulfonyl(C₁-C₄) alkyl, C₃-C₆ cycloalkyl, C₃-C₆ cycloalkyl(C₁-C₄)alkyl-, or tetrahydrofuranyl; R⁹ is C₁-C₄alkyl, C₁-C₄haloalkyl, C₁-C₄ alkyl-O—CH₂—, C₁-C₄ haloalkyl-O—CH₂—, C₃-C₆ cycloalkyl, C₃-C₆ cycloalkyl-CH₂—, alkyl-S—CH₂—, C₁-C₄ alkyl-S(O)—CH₂—, or C₁-C₄ alkyl-S(O₂)—CH₂; each Z is independently halogen, C₁-C₁₂ alkyl or C₁-C₁₂ alkyl substituted by one to five R¹², nitro, C₁-C₁₂ alkoxy or C₁-C₁₂ alkoxy substituted by one to five R¹² cyano, C₁-C₁₂alkylsulfinyl, C₁-C₁₂alkylsulfonyl, C₁-C₁₂haloalkylsulfinyl, C₁-C₁₂haloalkylsulfonyl, hydroxyl or thiol; each R¹² is halogen, cyano, nitro, hydroxy, C₁-C₈alkoxy-, C₁-C₈haloalkoxy-, mercapto, C₁-C₈alkylthio-, or C₁-C₈haloalkylthio; and k is 0, 1, 2 or 3. The methods are preferably for the control of Anthonomus grandis.

The present invention relates to a method of controlling insects, in particular insects of the family Curculionidae, and in particular in cotton.

Insects of the Curculionidae family (commonly known as weevils) are almost entirely plant feeders. Several members of this family, in particular members of the genus Anthonomus, are known to be important agronomic pests in a variety of commercially important flowering crops and flowering ornamental plants. Weevils are known to attack, inter alia, cotton, apple, hazelnut, oil seed rape, strawberry and pepper crops.

Benchmark active ingredients which have so far been used to control members of the Curculionidae, in particular Anthonomus, suffer several disadvantages. One of these, endosulfan, has been banned in over 80 countries due to its threat to human health and the environment. Pyrethroids such as cyfluthrin and betacyfluthrin are less effective now due to the appearance of resistance. Thus, there is a clear need to find insecticides which can be used to control Curculionidae, in particular Anthonomus.

Compounds that are insecticidally, acaricidally, nematicidally and/or moluscicidally active by antagnonism of the gamma-aminobutyric acid (GABA)-gated chloride channel, and which comprise a partially saturated heterocycle that is substituted by a haloalkyl substituent and one or two optionally substituted aromatic or heteroaromatic rings, represent a new class of pesticides that are described for example in Ozoe et al. Biochemical and Biophysical Research Communications, 391 (2010) 744-749. Compounds from this class are broadly described in WO 2005/085216 (EP1731512), WO 2007/123853, WO 2007/075459, WO 2009/002809, WO 2008/019760, WO 2008/122375, WO 2008/128711, WO 2009/097992, WO 2010/072781, WO 2010/072781, WO 2008/126665, WO 2007/125984, WO 2008/130651, JP2008110971, JP2008133273, JP2009108046, WO 2009/022746, WO 2009/022746, WO 2010/032437, WO 2009/080250, WO 2010/020521, WO 2010/025998, WO 2010/020522, WO 2010/084067, WO 2010/086225, WO 2010/149506 and WO 2010/108733.

It has now surprisingly been found that particular insecticides from this new class of gamma-aminobutyric acid (GABA)-gated chloride channel antagonists (disclosed in e.g. WO 2009/080250, WO 2010/020522, WO 2010/149506, WO 2011/101229 and WO 2012/045700) are highly effective at controlling Anthonomus.

These compounds therefore represent an important new solution for safeguarding crops of useful plants, particularly cotton crops, from insects from the family Curculionidae, particularly where the insects are resistant to current methods.

In a first aspect the invention provides a method comprising applying to a crop of cotton plants, the locus thereof, or propagation material thereof, a compound of formula I

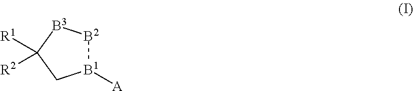

wherein -B¹-B²-B³- is —C═N—O—, —C═N—CH₂—, or —N—CH₂—CH₂—;
R¹ is trifluoromethyl, difluoromethyl or chlorodifluoromethyl;
R² is group X

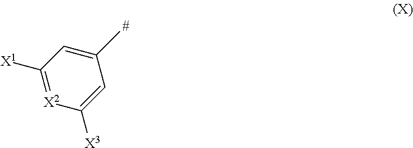

X² is C—X⁶ or nitrogen;
X¹, X³ and X⁶ are independently hydrogen, halogen or trihalomethyl, wherein at least one of X¹, X³ and
X⁶ is not hydrogen;
A is selected from A1 to A5

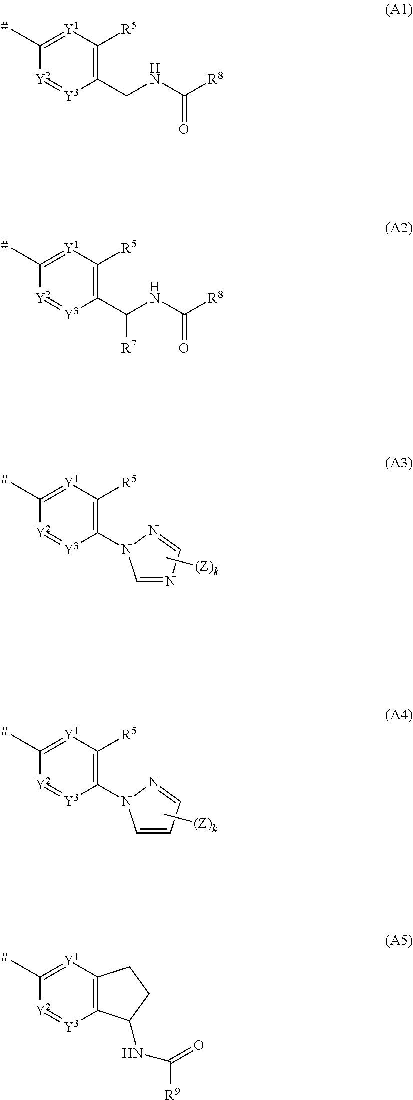

Y¹ is C—R⁶, CH or nitrogen;
Y² and Y³ are independently CH or nitrogen;
wherein no more than two of Y¹, Y² and Y³ are nitrogen and wherein Y² and Y³ are not both nitrogen; R⁵ is hydrogen, halogen, cyano, nitro, NH₂, C₁-C₄alkyl, C₁-C₄haloalkyl, C₃-C₅cycloalkyl, C₃-C₅halocycloalkyl, C₁-C₂alkoxy, or C₁-C₂haloalkoxy; provided that when A is A3 or A4 R⁵ is not hydrogen;
R⁶ when present together with R⁵ forms a —CH═CH—CH═CH— bridge;
R⁷ is C₁-C₄alkyl;
R⁸ is C₁-C₄alkyl, C₁-C₄haloalkyl, C₁-C₄alkoxy(C₁-C₄)alkyl, C₁-C₄alkylthio(C₁-C₄)alkyl, C₁-C₄alkylsulfinyl(C₁-C₄)alkyl, C₁-C₄alkylsulfonyl(C₁-C₄)alkyl, C₃-C₆cycloalkyl, C₃-C₆cycloalkyl(C₁-C₄)alkyl-, or tetrahydrofuranyl;
R⁹ is C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂—, C₁-C₄alkyl-S—CH₂—, C₁-C₄alkyl-S(O)—CH₂—, or C₁-C₄alkyl-S(O₂)—CH₂;
each Z is independently halogen, C₁-C₁₂alkyl or C₁-C₁₂alkyl substituted by one to five R¹², nitro, C₁-C₁₂alkoxy or C₁-C₁₂alkoxy substituted by one to five R¹², cyano, C₁-C₁₂alkylsulfinyl, C₁-C₁₂alkylsulfonyl, C₁-C₁₂haloalkylsulfinyl, C₁-C₁₂haloalkylsulfonyl, hydroxyl or thiol;
each R¹² is halogen, cyano, nitro, hydroxy, C₁-C₈alkoxy-, C₁-C₈haloalkoxy-, mercapto, C₁-C₈alkylthio-, or C₁-C₈haloalkylthio; and
k is 0, 1, 2 or 3.

In a further aspect the invention provides a method of controlling and/or preventing infestation of insects of the family Curculionidae in cotton comprising applying to a crop of cotton plants, the locus thereof, or propagation material thereof, a compound that of formula I. The insects of the family Curculionidae may be those that are resistant to one or more other insecticides.

In a further aspect the invention provides a method of controlling and/or preventing infestation of insects of the family Curculionidae in a crop of useful plants comprising applying to a crop of useful plants, the locus thereof, or propagation material thereof, a compound of formula I. The insects of the family Curculionidae may be those that are resistant to one or more other insecticides.

In a further aspect the invention provides use of a compound of formula I for control of insects of the family Curculionidae in useful plants. The use may be for controlling Curculionidae that are resistant to one or more other insecticides.

In a further aspect the invention provides a method of controlling and/or preventing infestation of Anthonomus grandis in a crop of useful plants comprising applying to a crop of useful plants, the locus thereof, or propagation material thereof, a compound of formula I. The Anthonomus grandis may be those that are resistant to one or more other insecticides.

In a further aspect the invention provides use of a compound of formula I for control of Anthonomus grandis in useful plants. The use may be for controlling Anthonomus grandis that are resistant to one or more other insecticides.

In a further aspect the invention provides a method of controlling and/or preventing infestation of Anthonomus grandis in a crop of cotton comprising applying to a crop of cotton, the locus thereof, or propagation material thereof, a compound of formula I. The Anthonomus grandis may be those that are resistant to one or more other insecticides.

In a further aspect the invention provides use of a compound of formula I for control of Anthonomus grandis in cotton. The use may be for controlling Anthonomus grandis that are resistant to one or more other insecticides.

Protection of a crop in a plot can be achieved by applying one or more pesticides to only a designated area of the plot. The designated part of the plot is usually the outer most part of the plot, and is generally a border area of sufficient size that penetration of the insects/pests is prevented to the remaining part of the plot. The benefit of spraying the border areas of the plot include a greater ability to control pest infestation because said insects/pests predominantly approach the plot from the periphery of the plot (i.e. from the sides of the plot). There is also the benefit that a lower volume of active ingredient could be applied whilst still controlling the insects/pests pressure for the entire plot. Therefore, the designated plot part forms an outer border of the plot, which can uniformly encompass the remaining plot, but embodiments wherein the border is not uniform and wherein the border doesn't completely encompass the remaining plot is also envisaged provided the said insects/pests are controlled to an acceptable level in the plot. Further, the designated plot part doe not have to form the outer most part of the plot, but that is preferred to minimize the crop damage by the said insects/pests. The area of the designated plot is of such size that acceptable control of the insects/pests is achieved. A skilled person would understand that a plot containing a crop would be applied with a number of pesticides during its growth. The application of the pesticides to a designated plot can be applicable to all pesticide application, but would be more appropriate to those pesticides controlling those insects/pests that predominantly approach the plot from the periphery of the plot; accordingly, the remaining pesticides would be applied as normal, for example, throughout the plot. For example, pesticide A is for control of insects/pests that pests that predominantly approach the plot from the periphery of the plot while pesticide B is broad spectrum fungicide; therefore, pesticide A & B, either simultaneously or in any sequence, would be applied to the designated plot part (i.e. a border region), while pesticide B would applied be applied throughout the plot.

Accordingly, in a further aspect, the invention provides a method of protecting a plot of at least five hectares comprising a crop of useful plants, preferably cotton, susceptible to and/or under attack by insects/pests, preferably of the family Curculionidae, said method comprising applying to said plant and/or said insects/pests a compound of formula I in a pre-defined plot part that is between 0.5 to 20%, based on area, of the plot, provided said pre-defined plot part forms the outer border of the plot, wherein said insects/pests predominantly approach the plot from the periphery of the plot.

In a preferred embodiment, the pre-defined plot part that is applied with a compound of formula I is between 1 to 20%, based on area, of the plot, especially 5 to 15%. In an embodiment, the plot is between 5 to 10,000, such as at most 50 to 5,000, especially 100 to 1,000, particularly 150 to 500, hectares.

In a further aspect the invention provides a method for obtaining regulatory approval for the use of one or more of a compound of formula I to control, preferably in cotton, an insect of the family Curculionidae, in particular the species Anthonomus grandis, Conotrachelus nenuphar, Ceutorhynchus spp., or Curculio nucum, preferably Anthonomus grandis, comprising at least one step of referring to, submitting or relying on biological data showing that said active ingredient reduces insect pressure.

The compounds of the invention may exist in different geometric or optical isomers or tautomeric forms. This invention covers all such isomers and tautomers and mixtures thereof in all proportions as well as isotopic forms such as deuterated compounds. The compounds of the invention may contain one or more asymmetric carbon atoms, for example, at the C(#2)#3 group, and may exist as enantiomers (or as pairs of diastereoisomers) or as mixtures of such. Reference to compounds of the invention also includes reference to salts and N-oxides.

The preferences for B¹, B², B³, R¹, R², Y¹, Y², Y³, R⁵, R⁶, R⁷, R⁸, R⁹, R¹², X¹, X², X³, X⁶, A and k are, in any combination, as described below.

Preferably R¹ is trifluoromethyl, or chlorodifluoromethyl, most preferably trifluoromethyl.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen. Preferably at least two of X¹, X³ and X⁶ are chloro, bromo or trifluoromethyl.

In one group of compounds R² is 3,5-dichlorophenyl-, 3-chloro-4-fluorophenyl-, 3-fluoro-4-chlorophenyl-, 3,4-dichlorophenyl-, 3-chloro-4-bromophenyl-, 3,5-dichloro-4-fluorophenyl-, 3,4,5-trichlorophenyl-, 3,5-dichloro-4-iodophenyl-, 3,4,5-trifluorophenyl-, 3-chloro-5-bromophenyl-, 3-chloro-5-fluorophenyl-, 3-chloro-5-(trifluoromethyl)phenyl-, 3,4-dichloro-5-(trifluoromethyl)phenyl-, 3,5-bis(trifluoromethyl)phenyl-, 4-chloro-3,5-bis(trifluoromethyl)phenyl-, 3-(trifluoromethyl)phenyl-, 2,6-dichloro-4-pyridyl-, 2,6-bis(trifluoromethyl)-4-pyridyl-, 3-bromo-5-(trifluoromethyl)phenyl-, more preferably 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, more preferably R² is 3,5-dichloro-phenyl, 3,5-dichloro-4-fluorophenyl- or 3,4,5-trichloro-phenyl, most preferably 3,5-dichloro-phenyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Most preferably Y¹ is CH, Y² is CH, and Y³ is CH.

Preferably R⁵ is hydrogen, chloro, bromo, fluoro, trifluoromethyl, methyl, ethyl, methoxy, nitro, trifluoromethoxy, cyano, cyclopropyl, more preferably R⁵ is hydrogen, chloro, bromo, fluoro, trifluoromethyl, methyl, ethyl, nitro, cyano, cyclopropyl, most preferably R⁵ is hydrogen, cyano, chloro, bromo, fluoro, methyl, or trifluoromethyl.

Preferably R⁸ is C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂—, C₁-C₄alkyl-S(O)—CH₂—, C₁-C₄alkyl-S(O₂)—CH₂—, more preferably C₁-C₄alkyl, C₁-C₄alkyl-O—CH₂—, C₁-C₄alkyl-S—CH₂—, C₁-C₄alkyl-SO—CH₂—, C₁-C₄alkyl-SO₂—CH₂—, C₃-C₄cycloalkyl, or C₃-C₄cycloalkyl-CH₂—, most preferably R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—.

Preferably R⁹ is C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂—, C₁-C₄alkyl-S(O)—CH₂—, C₁-C₄alkyl-S(O₂)—CH₂—, more preferably C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂-, more preferably C₁-C₄alkyl, C₁-C₄haloalkyl or C₃-C₄cycloalkyl, more preferably methyl, ethyl, n-propyl, CF₃CH₂— or cyclopropyl, even more preferably ethyl, CF₃CH₂— or cyclopropyl.

Preferably each Z is independently halogen, cyano, C₁-C₄alkyl, C₁-C₄haloalkyl, C₁-C₄alkoxy, or C₁-C₄haloalkoxy, most preferably each Z is independently hydrogen, cyano, halogen, methyl, halomethyl, methoxy or halomethoxy, most preferably cyano or trifluoromethyl.

Each R¹² is preferably bromo, chloro, fluoro, methoxy, or methylthio, most preferably chloro, fluoro, or methoxy.

Preferably k is 0 or 1.

In one group of compounds -B¹-B²-B³- is —C═N—O—.

In another group of compounds -B¹-B²-B³- is —C═N—CH₂—.

In another group of compounds -B¹-B²-B³- is —N—CH₂—CH₂—.

In another group of compounds Y¹ is C—R⁶ and R⁶ together with R⁵ forms a —CH═CH—CH═CH— bridge.

In one embodiment the compound of formula I is a compound of formula IA

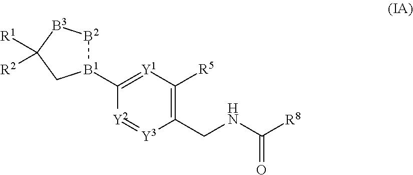

wherein B¹, B², B³, R¹, R², Y¹, Y², Y³, R⁵ and R⁸ are as defined for a compound of formula I.

In compounds of formula IA preferred definitions of B¹, B², B³, R¹, R², Y¹, Y², Y³, R⁵ and R⁸ are, in any combination, as set out below:

Preferably R¹ is trifluoromethyl, or chlorodifluoromethyl, most preferably trifluoromethyl.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen. Preferably at least two of X¹, X³ and X⁶ are chloro, bromo or trifluoromethyl.

In one group of compounds R² is 3,5-dichlorophenyl-, 3-chloro-4-fluorophenyl-, 3-fluoro-4-chlorophenyl-, 3,4-dichlorophenyl-, 3-chloro-4-bromophenyl-, 3,5-dichloro-4-fluorophenyl-, 3,4,5-trichlorophenyl-, 3,5-dichloro-4-iodophenyl-, 3,4,5-trifluorophenyl-, 3-chloro-5-bromophenyl-, 3-chloro-5-fluorophenyl-, 3-chloro-5-(trifluoromethyl)phenyl-, 3,4-dichloro-5-(trifluoromethyl)phenyl-, 3,5-bis(trifluoromethyl)phenyl-, 4-chloro-3,5-bis(trifluoromethyl)phenyl-, 3-(trifluoromethyl)phenyl-, 2,6-dichloro-4-pyridyl-, 2,6-bis(trifluoromethyl)-4-pyridyl-, 3-bromo-5-(trifluoromethyl)phenyl-, more preferably 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, more preferably R² is 3,5-dichloro-phenyl or 3,4,5-trichloro-phenyl, most preferably 3,5-dichloro-phenyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Most preferably Y¹ is CH, Y² is CH, and Y³ is CH.

Preferably R⁵ is hydrogen, chloro, bromo, fluoro, trifluoromethyl, methyl, ethyl, methoxy, nitro, trifluoromethoxy, cyano, cyclopropyl, more preferably R⁵ is hydrogen, chloro, bromo, fluoro, trifluoromethyl, methyl, ethyl, nitro, cyano, cyclopropyl, more preferably R⁵ is hydrogen, cyano, chloro, bromo, fluoro, methyl, or trifluoromethyl, even more preferably hydrogen, chloro, bromo, methyl or trifluoromethyl, most preferably chloro, bromo, fluoro or methyl.

Preferably R⁸ is C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂—, C₁-C₄alkyl-S(O)—CH₂—, C₁-C₄alkyl-S(O₂)—CH₂—, more preferably C₁-C₄alkyl, C₁-C₄alkyl-O—CH₂—, C₁-C₄alkyl-S—CH₂—, C₁-C₄alkyl-SO—CH₂—, C₁-C₄alkyl-SO₂—CH₂—, C₃-C₄cycloalkyl, or C₃-C₄cycloalkyl-CH₂—, more preferably R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, most preferably n-propyl or cyclopropyl.

In one group of compounds of formula IA -B¹-B²-B³- is —C═N—O—.

In another group of compounds of formula IA -B¹-B²-B³- is —C═N—O— and Y¹ is CH, Y² is CH, Y³ is CH.

In another group of compounds of formula IA -B¹-B²-B³- is —C═N—O—, Y¹ is CH, Y² is CH, Y³ is CH and R¹ is CF₃.

In another group of compounds of formula IA -B¹-B²-B³- is —C═N—O—, Y¹ is CH, Y² is CH, Y³ is CH, R¹ is CF₃ and R⁵ is chloro, bromo, fluoro or methyl.

In another group of compounds of formula IA -B¹-B²-B³- is —C═N—O—, Y¹ is CH, Y² is CH, Y³ is CH, R¹ is CF₃, and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IA -B¹-B²-B³- is —C═N—O—, Y¹ is CH, Y² is CH, Y³ is CH, R¹ is CF₃, R⁵ is chloro, bromo, fluoro or methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IA -B¹-B²-B³- is —C═N—O—, Y¹ is CH, Y² is CH, Y³ is CH, R¹ is CF₃, R⁵ is chloro, bromo, fluoro or methyl, R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichloro-phenyl-.

In one group of compounds of formula IA -B¹-B²-B³- is —C═N—CH₂—.

In another group of compounds of formula IA -B¹-B²-B³- is —C═N—CH₂— and Y¹ is CH, Y² is CH, Y³ is CH.

In another group of compounds of formula IA -B¹-B²-B³- is —C═N—CH₂—, Y¹ is CH, Y² is CH, Y³ is CH and R¹ is CF₃.

In another group of compounds of formula IA -B¹-B²-B³- is —C═N—CH₂—, Y¹ is CH, Y² is CH, Y³ is CH, R¹ is CF₃ and R⁵ is chloro, bromo, fluoro or methyl.

In another group of compounds of formula IA -B¹-B²-B³- is —C═N—CH₂—, Y¹ is CH, Y² is CH, Y³ is CH, R¹ is CF₃, and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IA -B¹-B²-B³- is —C═N—CH₂—, Y¹ is CH, Y² is CH, Y³ is CH, R¹ is CF₃, R⁵ is chloro, bromo, fluoro or methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IA -B¹-B²-B³- is —C═N—CH₂—, Y¹ is CH, Y² is CH, Y³ is CH, R¹ is CF₃, R⁵ is chloro, bromo, fluoro or methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IA -B¹-B²-B³- is —N—CH₂—CH₂—.

In another group of compounds of formula IA -B¹-B²-B³- is —N—CH₂—CH₂—, Y¹ is CH, Y² is CH, Y³ is CH.

In another group of compounds of formula IA -B¹-B²-B³- is —N—CH₂—CH₂—, Y¹ is CH, Y² is CH, Y³ is CH and R¹ is CF₃.

In another group of compounds of formula IA -B¹-B²-B³- is —N—CH₂—CH₂—, Y¹ is CH, Y² is CH, Y³ is CH, R¹ is CF₃ and R⁵ is chloro, bromo, fluoro or methyl.

In another group of compounds of formula IA -B¹-B²-B³- is —N—CH₂—CH₂—, Y¹ is CH, Y² is CH,

Y³ is CH, R¹ is CF₃, and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IA -B¹-B²-B³- is —N—CH₂—CH₂—, Y¹ is CH, Y² is CH, Y³ is CH, R¹ is CF₃, R⁵ is chloro, bromo, fluoro or methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂, preferably n-propyl or cyclopropyl.

In another group of compounds of formula IA -B¹-B²-B³- is —N—CH₂—CH₂—, Y¹ is CH, Y² is CH, Y³ is CH, R¹ is CF₃, R⁵ is chloro, bromo, fluoro or methyl, R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In another group of compounds of formula IA R⁵ is chloro, bromo, fluoro or methyl and R⁸ methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IA R⁵ is chloro, bromo, fluoro or methyl; R⁸ methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, R¹ is CF₃, -B¹-B²-B³ is —C═N—O— or —C═N—CH₂—, Y¹, Y² and Y³ are CH, and R⁵ is chloro or methyl.

In a further preferred embodiment of compounds of formula IA -B¹-B²-B³- is —C═N—O—, R¹ is trifluoromethyl, Y¹, Y² and Y³ are CH, R⁵ is chloro, and R⁸ is n-propyl.

In a further preferred embodiment of compounds of formula IA -B¹-B²-B³- is —C═N—O—, R¹ is trifluoromethyl, Y¹, Y² and Y³ are CH, R⁵ is chloro, and R⁸ is n-propyl.

In a further preferred embodiment of compounds of formula IA -B¹-B²-B³- is —C═N—O—, R¹ is trifluoromethyl, R² is 3-bromo-5-trifluoromethylphenyl, Y¹, Y² and Y³ are CH and R⁸ is n-propyl.

In a further preferred embodiment of compounds of formula IA -B¹-B²-B³- is —C═N—O—, R¹ is trifluoromethyl, R² is 3-bromo-5-trifluoromethylphenyl, Y¹, Y² and Y³ are CH and R⁵ is chloro.

In a further preferred embodiment of compounds of formula IA -B¹-B²-B³- is —C═N—O—, R¹ is trifluoromethyl, R² is 3-bromo-5-trifluoromethylphenyl, Y¹, Y² and Y³ are CH, R⁵ is chloro, and R⁸ is n-propyl.

In one embodiment the compound of formula I is a compound of formula IB

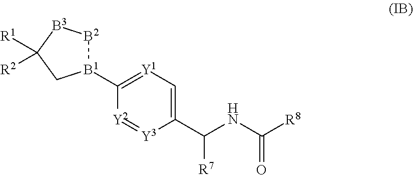

wherein B¹, B², B³, R¹, R², Y¹, Y², Y³, R⁷ and R⁸ are as defined for a compound of formula I.

In compounds of formula IB preferred definitions of B¹, B², B³, R¹, R², Y¹, Y², Y³, R⁷ and R⁸ are, in any combination, as set out below.

Preferably R¹ is trifluoromethyl, or chlorodifluoromethyl, most preferably trifluoromethyl.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen. Preferably at least two of X¹, X³ and X⁶ are chloro, bromo or trifluoromethyl.

In one group of compounds R² is 3,5-dichlorophenyl-, 3-chloro-4-fluorophenyl-, 3-fluoro-4-chlorophenyl-, 3,4-dichlorophenyl-, 3-chloro-4-bromophenyl-, 3,5-dichloro-4-fluorophenyl-, 3,4,5-trichlorophenyl-, 3,5-dichloro-4-iodophenyl-, 3,4,5-trifluorophenyl-, 3-chloro-5-bromophenyl-, 3-chloro-5-fluorophenyl-, 3-chloro-5-(trifluoromethyl)phenyl-, 3,4-dichloro-5-(trifluoromethyl)phenyl-, 3,5-bis(trifluoromethyl)phenyl-, 4-chloro-3,5-bis(trifluoromethyl)phenyl-, 3-(trifluoromethyl)phenyl-, 2,6-dichloro-4-pyridyl-, 2,6-bis(trifluoromethyl)-4-pyridyl-, 3-bromo-5-(trifluoromethyl)phenyl-, more preferably 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, more preferably R² is 3,5-dichloro-phenyl or 3,4,5-trichloro-phenyl, most preferably 3,5-dichloro-phenyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Most preferably Y¹ is CH, Y² is CH, and Y³ is CH.

Preferably R⁷ is methyl.

Preferably R⁸ is C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂—, C₁-C₄alkyl-S(O)—CH₂—, C₁-C₄alkyl-S(O₂)—CH₂—, more preferably C₁-C₄alkyl, C₁-C₄alkyl-O—CH₂—, C₁-C₄alkyl-S—CH₂—, C₁-C₄alkyl-SO—CH₂—, C₁-C₄alkyl-SO₂—CH₂—, C₃-C₄cycloalkyl, or C₃-C₄cycloalkyl-CH₂—, more preferably R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, most preferably n-propyl or cyclopropyl.

In one group of compounds of formula IB -B¹-B²-B³- is —C═N—O—.

In another group of compounds of formula IB -B¹-B²-B³- is —C═N—O— and Y¹ is CH, Y² is CH, Y³ is CH.

In another group of compounds of formula IB -B¹-B²-B³- is —C═N—O—, Y¹ is CH, Y² is CH, Y³ is CH and R¹ is CF₃.

In another group of compounds of formula IB -B¹-B²-B³- is —C═N—O—, Y¹ is CH, Y² is CH, Y³ is CH, R¹ is CF₃ and R⁷ is methyl.

In another group of compounds of formula IB -B¹-B²-B³- is —C═N—O—, Y¹ is CH, Y² is CH, Y³ is CH, R¹ is CF₃, and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of -B¹-B²-B³- is —C═N—O—, Y¹ is CH, Y² is CH, Y³ is CH, R¹ is CF₃, R⁷ is methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IB -B¹-B²-B³- is —C═N—O—, Y¹ is CH, Y² is CH, Y³ is CH, R¹ is CF₃, R⁷ is methyl, R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂, preferably n-propyl or cyclopropyl, —and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IB -B¹-B²-B³- is —C═N—CH₂—.

In another group of compounds of formula IB -B¹-B²-B³- is —C═N—CH₂— and Y¹ is CH, Y² is CH, Y³ is CH.

In another group of compounds of formula IB -B¹-B²-B³- is —C═N—CH₂—, Y¹ is CH, Y² is CH, Y³ is CH and R¹ is CF₃.

In another group of compounds of formula IB -B¹-B²-B³- is —C═N—CH₂—, Y¹ is CH, Y² is CH, Y³ is CH, R¹ is CF₃ and R⁷ is methyl.

In another group of compounds of formula IB -B¹-B²-B³- is —C═N—CH₂—, Y¹ is CH, Y² is CH, Y³ is CH, R¹ is CF₃, and R⁸ is methyl, ethyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably cyclopropyl.

In another group of compounds of formula IB -B¹-B²-B³- is —C═N—CH₂—, Y¹ is CH, Y² is CH, Y³ is CH, R¹ is CF₃, R⁷ is methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IB -B¹-B²-B³- is —C═N—CH₂—, Y¹ is CH, Y² is CH, Y³ is CH, R¹ is CF₃, R⁷ is methyl, R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IB -B¹-B²-B³- is —N—CH₂—CH₂—.

In another group of compounds of formula IB -B¹-B²-B³- is —N—CH₂—CH₂— and Y¹ is CH, Y² is CH, Y³ is CH.

In another group of compounds of formula IB -B¹-B²-B³- is —N—CH₂—CH₂—, Y¹ is CH, Y² is CH, Y³ is CH and R¹ is CF₃.

In another group of compounds of formula IB -B¹-B²-B³- is —N—CH₂—CH₂—, Y¹ is CH, Y² is CH, Y³ is CH, R¹ is CF₃ and R⁷ is methyl.

In another group of compounds of formula IB -B¹-B²-B³- is —N—CH₂—CH₂—, Y¹ is CH, Y² is CH, Y³ is CH, R¹ is CF₃, and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IB -B¹-B²-B³- is —N—CH₂—CH₂—, Y¹ is CH, Y² is CH, Y³ is CH, R¹ is CF₃, R⁷ is methyl and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IB -B¹-B²-B³- is —N—CH₂—CH₂—, Y¹ is CH, Y² is CH, Y³ is CH, R¹ is CF₃, R⁷ is methyl, R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In another group of compounds of formula IB R⁷ is methyl, and R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl.

In another group of compounds of formula IB R⁷ is methyl, R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—, preferably n-propyl or cyclopropyl, R¹ is CF₃, -B¹-B²-B³- is —C═N—O—, —C═N—CH₂— or —N—CH₂—CH₂—, and Y¹, Y² and Y³ are CH.

In a further preferred embodiment of compounds of formula IB -B¹-B²-B³- is —C═N—O—, R¹ is trifluoromethyl, Y¹, Y² and Y³ are CH; R⁵ is chloro, R⁷ is methyl and R⁸ is n-propyl.

In a further preferred embodiment of compounds of formula IB -B¹-B²-B³- is —C═N—O—, R¹ is trifluoromethyl, Y¹, Y² and Y³ are CH; R⁵ is chloro, R⁷ is methyl and R⁸ is n-propyl.

In a further preferred embodiment of compounds of formula IB -B¹-B²-B³- is —C═N—O—, R¹ is trifluoromethyl, R² is 3-bromo-5-trifluoromethylphenyl, Y¹, Y² and Y³ are CH, R⁷ is methyl and R⁸ is n-propyl.

In a further preferred embodiment of compounds of formula IB -B¹-B²-B³- is —C═N—O—, R¹ is trifluoromethyl, R² is 3-bromo-5-trifluoromethylphenyl, Y¹, Y² and Y³ are CH, R⁵ is chloro and R⁷ is methyl.

In a further preferred embodiment of compounds of formula IB -B¹-B²-B³- is —C═N—O—, R¹ is trifluoromethyl, R² is 3-bromo-5-trifluoromethylphenyl, Y¹, Y² and Y³ are CH, R⁵ is chloro, R⁷ is methyl and R⁸ is n-propyl.

In one embodiment the compound of formula I is a compound of formula IC

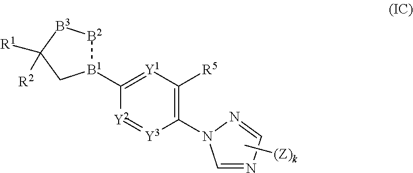

wherein B¹, B², B³, R¹, R², Y¹, Y², Y³, R⁵, Z and k are as defined for a compound of formula I.

In compounds of formula IC preferred definitions of B¹, B², B³, R¹, R², Y¹, Y², Y³, R⁵, Z and k are, in any combination, as set out below.

Preferably R¹ is trifluoromethyl, or chlorodifluoromethyl, most preferably trifluoromethyl.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen. Preferably at least two of X¹, X³ and X⁶ are chloro, bromo or trifluoromethyl.

In one group of compounds R² is 3,5-dichlorophenyl-, 3-chloro-4-fluorophenyl-, 3-fluoro-4-chlorophenyl-, 3,4-dichlorophenyl-, 3-chloro-4-bromophenyl-, 3,5-dichloro-4-fluorophenyl-, 3,4,5-trichlorophenyl-, 3,5-dichloro-4-iodophenyl-, 3,4,5-trifluorophenyl-, 3-chloro-5-bromophenyl-, 3-chloro-5-fluorophenyl-, 3-chloro-5-(trifluoromethyl)phenyl-, 3,4-dichloro-5-(trifluoromethyl)phenyl-, 3,5-bis(trifluoromethyl)phenyl-, 4-chloro-3,5-bis(trifluoromethyl)phenyl-, 3-(trifluoromethyl)phenyl-, 2,6-dichloro-4-pyridyl-, 2,6-bis(trifluoromethyl)-4-pyridyl-, 3-bromo-5-(trifluoromethyl)phenyl-, more preferably 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, more preferably R² is 3,5-dichloro-phenyl or 3,4,5-trichloro-phenyl, most preferably 3,5-dichloro-phenyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Most preferably Y¹ is CH, Y² is CH, and Y³ is CH.

Preferably R⁵ is chloro, bromo, fluoro, trifluoromethyl, methyl, ethyl, methoxy, nitro, trifluoromethoxy, cyano, cyclopropyl, more preferably R⁵ is chloro, bromo, fluoro, trifluoromethyl, methyl, ethyl, nitro, cyano, cyclopropyl, even more preferably R⁵ is cyano, chloro, bromo, fluoro, methyl, or trifluoromethyl, most preferably cyano.

Preferably each Z is independently halogen, cyano, C₁-C₄alkyl, C₁-C₄haloalkyl, C₁-C₄alkoxy, or C₁-C₄haloalkoxy, more preferably each Z is independently hydrogen, cyano, halogen, methyl, halomethyl, methoxy or halomethoxy, most preferably cyano or trifluoromethyl.

Preferably k is 0 or 1.

In one group of compounds of formula IC -B¹-B²-B³- is —C═N—O—.

In another group of compounds of formula IC -B¹-B²-B³- is —C═N—O— and Y¹ is CH, Y² is CH, Y³ is CH.

In another group of compounds of formula IC -B¹-B²-B³- is —C═N—O—, Y¹ is CH, Y² is CH, Y³ is CH and R¹ is CF₃.

In another group of compounds of formula IC -B¹-B²-B³- is —C═N—O—, Y¹ is CH, Y² is CH, Y³ is CH, R¹ is CF₃ and R⁵ is cyano.

In another group of compounds of formula IC -B¹-B²-B³- is —C═N—O—, Y¹ is CH, Y² is CH, Y³ is CH, R¹ is CF₃, and k is 0 or k is 1 and Z is cyano or trifluoromethyl.

In another group of compounds of formula IC -B¹-B²-B³- is —C═N—O—, Y¹ is CH, Y² is CH, Y³ is CH, R¹ is CF₃, R⁵ is cyano and k is 0 or k is 1 and Z is cyano or trifluoromethyl.

In another group of compounds of formula IC -B¹-B²-B³- is —C═N—O—, Y¹ is CH, Y² is CH, Y³ is CH, R¹ is CF₃, R⁵ is cyano and k is 0 or k is 1 and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IC -B¹-B²-B³- is —C═N—CH₂—.

In another group of compounds of formula IC -B¹-B²-B³- is —C═N—CH₂—, Y¹ is CH, Y² is CH, Y³ is CH.

In another group of compounds of formula IC -B¹-B²-B³- is —C═N—CH₂—, Y¹ is CH, Y² is CH, Y³ is CH and R¹ is CF₃.

In another group of compounds of formula IC -B¹-B²-B³- is —C═N—CH₂—, Y¹ is CH, Y² is CH, Y³ is CH, R¹ is CF₃ and R⁵ is cyano.

In another group of compounds of formula IC -B¹-B²-B³- is —C═N—CH₂—, Y¹ is CH, Y² is CH, Y³ is CH, R¹ is CF₃, and k is 0 or k is 1 and Z is cyano or trifluoromethyl.

In another group of compounds of formula IC -B¹-B²-B³- is —C═N—CH₂—, Y¹ is CH, Y² is CH, Y³ is CH, R¹ is CF₃, R⁵ is cyano and k is 0 or k is 1 and Z is cyano or trifluoromethyl.

In another group of compounds of formula IC -B¹-B²-B³- is —C═N—CH₂—, Y¹ is CH, Y² is CH, Y³ is CH, R¹ is CF₃, R⁵ is cyano and k is 0 or k is 1 and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IC -B¹-B²-B³- is —N—CH₂—CH₂—.

In another group of compounds of formula IC -B¹-B²-B³- is —N—CH₂—CH₂— and Y¹ is CH, Y² is CH, Y³ is CH.

In another group of compounds of formula IC -B¹-B²-B³- is —N—CH₂—CH₂—, Y¹ is CH, Y² is CH, Y³ is CH and R¹ is CF₃.

In another group of compounds of formula IC -B¹-B²-B³- is —N—CH₂—CH₂—, Y¹ is CH, Y² is CH, Y³ is CH, R¹ is CF₃ and R⁵ is cyano.

In another group of compounds of formula IC -B¹-B²-B³- is —N—CH₂—CH₂—, Y¹ is CH, Y² is CH, Y³ is CH, R¹ is CF₃, and k is 0 or k is 1 and Z is cyano or trifluoromethyl.

In another group of compounds of formula IC -B¹-B²-B³- is —N—CH₂—CH₂—, Y¹ is CH, Y² is CH, Y³ is CH, R¹ is CF₃, R⁵ is cyano and k is 0 or k is 1 and Z is cyano or trifluoromethyl.

In another group of compounds of formula IC -B¹-B²-B³- is —N—CH₂—CH₂—, Y¹ is CH, Y² is CH, Y³ is CH, R¹ is CF₃, R⁵ is cyano and k is 0 or k is 1 and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In another group of compounds of formula IC R⁵ is cyano, k is 0 or 1 and Z is cyano or trifluoromethyl.

In another group of compounds of formula IC R⁵ is cyano, R¹ is CF₃, -B¹-B²-B³- is —C═N—O— or —C═N—CH₂—, Y¹, Y² and Y³ are CH, and k is 0.

In one embodiment the compound of formula I is a compound of formula ID

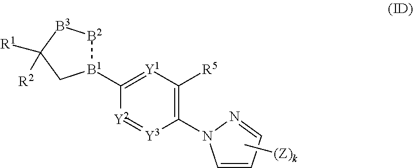

wherein B¹, B², B³, R¹, R², Y¹, Y², Y³, R⁵ Z and k are as defined for a compound of formula I

In compounds of formula ID preferred definitions of B¹, B², B³, R¹, R², Y¹, Y², Y³, R⁵ Z and k are, in any combination, as set out below.

Preferably R¹ is trifluoromethyl, or chlorodifluoromethyl, most preferably trifluoromethyl.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen. Preferably at least two of X¹, X³ and X⁶ are chloro, bromo or trifluoromethyl.

In one group of compounds R² is 3,5-dichlorophenyl-, 3-chloro-4-fluorophenyl-, 3-fluoro-4-chlorophenyl-, 3,4-dichlorophenyl-, 3-chloro-4-bromophenyl-, 3,5-dichloro-4-fluorophenyl-, 3,4,5-trichlorophenyl-, 3,5-dichloro-4-iodophenyl-, 3,4,5-trifluorophenyl-, 3-chloro-5-bromophenyl-, 3-chloro-5-fluorophenyl-, 3-chloro-5-(trifluoromethyl)phenyl-, 3,4-dichloro-5-(trifluoromethyl)phenyl-, 3,5-bis(trifluoromethyl)phenyl-, 4-chloro-3,5-bis(trifluoromethyl)phenyl-, 3-(trifluoromethyl)phenyl-, 2,6-dichloro-4-pyridyl-, 2,6-bis(trifluoromethyl)-4-pyridyl-, 3-bromo-5-(trifluoromethyl)phenyl-, more preferably 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, more preferably R² is 3,5-dichloro-phenyl or 3,4,5-trichloro-phenyl, most preferably 3,5-dichloro-phenyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Most preferably Y¹ is CH, Y² is CH, and Y³ is CH.

Preferably R⁵ is chloro, bromo, fluoro, trifluoromethyl, methyl, ethyl, methoxy, nitro, trifluoromethoxy, cyano, cyclopropyl, more preferably R⁵ is chloro, bromo, fluoro, trifluoromethyl, methyl, ethyl, nitro, cyano, cyclopropyl, even more preferably R⁵ is cyano, chloro, bromo, fluoro, methyl, or trifluoromethyl, most preferably cyano.

Preferably each Z is independently halogen, cyano, C₁-C₄alkyl, C₁-C₄haloalkyl, C₁-C₄alkoxy, or C₁-C₄haloalkoxy, more preferably each Z is independently hydrogen, cyano, halogen, methyl, halomethyl, methoxy or halomethoxy, most preferably cyano or trifluoromethyl.

Preferably k is 0 or 1, more preferably 1. When k is 1 preferably Z is attached to the 4 position of the imidazole moiety as indicated below:

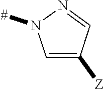

In one group of compounds of formula ID -B¹-B²-B³- is —C═N—O—.

In another group of compounds of formula ID -B¹-B²-B³- is —C═N—O— and Y¹ is CH, Y² is CH, Y³ is CH.

In another group of compounds of formula ID -B¹-B²-B³- is —C═N—O—, Y¹ is CH, Y² is CH, Y³ is CH and R¹ is CF₃.

In another group of compounds of formula ID -B¹-B²-B³- is —C═N—O—, Y¹ is CH, Y² is CH, Y³ is CH, R¹ is CF₃ and R⁵ is cyano.

In another group of compounds of formula ID -B¹-B²-B³- is —C═N—O—, Y¹ is CH, Y² is CH, Y³ is CH, R¹ is CF₃, and k is 0 or k is 1 and Z is cyano or trifluoromethyl.

In another group of compounds of formula ID -B¹-B²-B³- is —C═N—O—, Y¹ is CH, Y² is CH, Y³ is CH, R¹ is CF₃, R⁵ is cyano and k is 0 or k is 1 and Z is cyano or trifluoromethyl.

In another group of compounds of formula ID -B¹-B²-B³- is —C═N—O—, Y¹ is CH, Y² is CH, Y³ is CH, R¹ is CF₃, R⁵ is cyano and k is 0 or k is 1, preferably 1, and Z is cyano or trifluoromethyl, and when k is 1, Z is attached to the 4 position of the imidazole moiety.

In another group of compounds of formula ID -B¹-B²-B³- is —C═N—O—, Y¹ is CH, Y² is CH, Y³ is CH, R¹ is CF₃, R⁵ is cyano and k is 0 or k is 1 and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In another group of compounds of formula ID -B¹-B²-B³- is —C═N—O—, Y¹ is CH, Y² is CH, Y³ is CH, R¹ is CF₃, R⁵ is cyano and k is 0 or 1 or k is 1, preferably 1, and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, and when k is 1, Z is attached to the 4 position of the imidazole moiety.

In one group of compounds of formula ID -B¹-B²-B³- is —C═N—CH₂—.

In another group of compounds of formula ID -B¹-B²-B³- is —C═N—CH₂— and Y¹ is CH, Y² is CH, Y³ is CH.

In another group of compounds of formula ID -B¹-B²-B³- is —C═N—CH₂—, Y¹ is CH, Y² is CH, Y³ is CH and R¹ is CF₃.

In another group of compounds of formula ID -B¹-B²-B³- is —C═N—CH₂—, Y¹ is CH, Y² is CH, Y³ is CH, R¹ is CF₃ and R⁵ is cyano.

In another group of compounds of formula ID -B¹-B²-B³- is —C═N—CH₂—, Y¹ is CH, Y² is CH, Y³ is CH, R¹ is CF₃, and k is 0 or k is 1 and Z is cyano or trifluoromethyl.

In another group of compounds of formula ID -B¹-B²-B³- is —C═N—CH₂—, Y¹ is CH, Y² is CH, Y³ is CH, R¹ is CF₃, R⁵ is cyano and k is 0 or k is 1 and Z is cyano or trifluoromethyl.

In another group of compounds of formula ID -B¹-B²-B³- is —C═N—CH₂—, Y¹ is CH, Y² is CH, Y³ is CH, R¹ is CF₃, R⁵ is cyano and k is 0 or k is 1, preferably 1, and Z is cyano or trifluoromethyl, and when k is 1, Z is attached to the 4 position of the imidazole moiety.

In another group of compounds of formula ID -B¹-B²-B³- is —C═N—CH₂—, Y¹ is CH, Y² is CH, Y³ is CH, R¹ is CF₃, R⁵ is cyano and k is 0 or k is 1 and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In another group of compounds of formula ID -B¹-B²-B³- is —C═N—CH₂—, Y¹ is CH, Y² is CH, Y³ is CH, R¹ is CF₃, R⁵ is cyano and k is 0 or k is 1, preferably 1, and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, and when k is 1, Z is attached to the 4 position of the imidazole moiety.

In one group of compounds of formula ID -B¹-B²-B³- is —N—CH₂—CH₂—.

In another group of compounds of formula ID -B¹-B²-B³- is —N—CH₂—CH₂— and Y¹ is CH, Y² is CH, Y³ is CH.

In another group of compounds of formula ID -B¹-B²-B³- is —N—CH₂—CH₂—, Y¹ is CH, Y² is CH, Y³ is CH and R¹ is CF₃.

In another group of compounds of formula ID -B¹-B²-B³- is —N—CH₂—CH₂—, Y¹ is CH, Y² is CH, Y³ is CH, R¹ is CF₃ and R⁵ is cyano.

In another group of compounds of formula ID -B¹-B²-B³- is —N—CH₂—CH₂—, Y¹ is CH, Y² is CH, Y³ is CH, R¹ is CF₃, and k is 0 or k is 1 and Z is cyano or trifluoromethyl.

In another group of compounds of formula ID -B¹-B²-B³- is —N—CH₂—CH₂—, Y¹ is CH, Y² is CH,

Y³ is CH, R¹ is CF₃, R⁵ is cyano and k is 0 or k is 1 and Z is cyano or trifluoromethyl.

In another group of compounds of formula ID -B¹-B²-B³- is —N—CH₂—CH₂—, Y¹ is CH, Y² is CH, Y³ is CH, R¹ is CF₃, R⁵ is cyano and k is 0 or k is 1, preferably 1, and Z is cyano or trifluoromethyl, and when k is 1, Z is attached to the 4 position of the imidazole moiety.

In another group of compounds of formula ID -B¹-B²-B³- is —N—CH₂—CH₂—, Y¹ is CH, Y² is CH, Y³ is CH, R¹ is CF₃, R⁵ is cyano and k is 0 or k is 1 and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In another group of compounds of formula ID -B¹-B²-B³- is —N—CH₂—CH₂—, Y¹ is CH, Y² is CH, Y³ is CH, R¹ is CF₃, R⁵ is cyano and k is 0 or k is 1, preferably 1, and Z is cyano or trifluoromethyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, and when k is 1, Z is attached to the 4 position of the imidazole moiety.

In another group of compounds of formula D R⁵ is cyano, k is 0 or 1 and Z is cyano or trifluoromethyl.

In another group of compounds of formula ID R⁵ is cyano, k is 0 or 1, preferably 1, and Z is cyano or trifluoromethyl, and when k is 1, Z is attached to the 4 position of the imidazole moiety.

In another group of compounds of formula ID R⁵ is cyano, k is 0 or 1 and Z is cyano or trifluoromethyl, R¹ is CF₃, -B¹-B²-B³- is —C═N—O— or —C═N—CH₂—, Y¹, Y² and Y³ are CH.

In another group of compounds of formula ID R⁵ is cyano, k is 0 or 1, preferably 1, and Z is cyano or trifluoromethyl, R¹ is CF₃, -B¹-B²-B³- is —C═N—O— or —C═N—CH₂—, Y¹, Y² and Y³ are CH, and when k is 1, Z is attached to the 4 position of the imidazole moiety.

In one embodiment the compound of formula I is a compound of formula IE

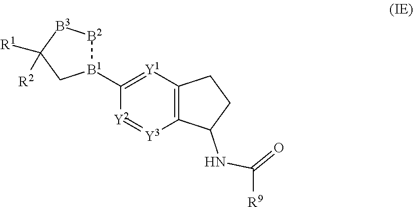

wherein B¹, B², B³, R¹, R², R⁹, Y¹, Y², and Y³ are as defined for a compound of formula I.

In compounds of formula ID preferred definitions of B¹, B², B³, R¹, R², R⁹, Y¹, Y², Y³, are, in any combination, as set out below.

Preferably R¹ is trifluoromethyl, or chlorodifluoromethyl, most preferably trifluoromethyl.

Preferably X¹, X³ and X⁶ are independently hydrogen, halogen or trifluoromethyl, wherein at least two of X¹, X³ and X⁶ are not hydrogen. More preferably X¹, X³ and X⁶ are independently hydrogen, chloro, bromo or trifluoromethyl, wherein at least two of X¹, X³ and X⁶ are not hydrogen. Preferably at least two of X¹, X³ and X⁶ are chloro, bromo or trifluoromethyl.

In one group of compounds R² is 3,5-dichlorophenyl-, 3-chloro-4-fluorophenyl-, 3-fluoro-4-chlorophenyl-, 3,4-dichlorophenyl-, 3-chloro-4-bromophenyl-, 3,5-dichloro-4-fluorophenyl-, 3,4,5-trichlorophenyl-, 3,5-dichloro-4-iodophenyl-, 3,4,5-trifluorophenyl-, 3-chloro-5-bromophenyl-, 3-chloro-5-fluorophenyl-, 3-chloro-5-(trifluoromethyl)phenyl-, 3,4-dichloro-5-(trifluoromethyl)phenyl-, 3,5-bis(trifluoromethyl)phenyl-, 4-chloro-3,5-bis(trifluoromethyl)phenyl-, 3-(trifluoromethyl)phenyl-, 2,6-dichloro-4-pyridyl-, 2,6-bis(trifluoromethyl)-4-pyridyl-, 3-bromo-5-(trifluoromethyl)phenyl-, more preferably 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-, more preferably R² is 3,5-dichloro-phenyl or 3,4,5-trichloro-phenyl, most preferably 3,5-dichloro-phenyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Most preferably Y¹ is CH, Y² is CH, and Y³ is CH.

Preferably R⁹ is C₁-C₄alkyl, C₁-C₄haloalkyl, C₁-C₄alkyl-O—CH₂—, C₁-C₄haloalkyl-O —CH₂—, C₃-C₆cycloalkyl, C₃-C₆cycloalkyl-CH₂—, C₁-C₄alkyl-S(O)—CH₂—, C₁-C₄alkyl-S(O₂)—CH₂—, more preferably C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂-, more preferably C₁-C₄alkyl, C₁-C₄haloalkyl or C₃-C₄cycloalkyl, more preferably methyl, ethyl, propyl, CF₃CH₂— or cyclopropyl, even more preferably ethyl, CF₃CH₂— or cyclopropyl.

Preferably Y¹ is CH, Y² is CH, Y³ is CH, or Y¹ is N, Y² is CH, Y³ is CH, or Y¹ is N, Y² is N, Y³ is CH, or Y¹ is CH, Y² is N, Y³ is CH, or Y¹ is CH, Y² is CH, Y³ is N. Preferably Y¹ is CH, Y² is CH, and Y³ is CH.

In one group of compounds of formula IE -B¹-B²-B³- is —C═N—O—.

In another group of compounds of formula IE -B¹-B²-B³- is —C═N—O— and Y¹ is CH, Y² is CH, Y³ is CH.

In another group of compounds of formula IE -B¹-B²-B³- is —C═N—O—, Y¹ is CH, Y² is CH, Y³ is CH and R¹ is CF₃.

In another group of compounds of formula IE -B¹-B²-B³- is —C═N—O—, Y¹ is CH, Y² is CH, Y³ is CH, R¹ is CF₃ and R⁹ is C₁-C₄alkyl.

In another group of compounds of formula IE -B¹-B²-B³- is —C═N—O—, Y¹ is CH, Y² is CH, Y³ is CH, R¹ is CF₃, and R⁹ is C₁-C₄haloalkyl.

In another group of compounds of formula IE -B¹-B²-B³- is —C═N—O—, Y¹ is CH, Y² is CH, Y³ is CH, R¹ is CF₃, R⁹ is C₃-C₄cycloalkyl.

In another group of compounds of formula IE -B¹-B²-B³- is —C═N—O—, Y¹ is CH, Y² is CH, Y³ is CH, R¹ is CF₃, R⁹ is C₁-C₄alkyl, C₁-C₄haloalkyl or C₃-C₄cycloalkyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IE -B¹-B²-B³- is —C═N—CH₂—.

In another group of compounds of formula IE -B¹-B²-B³- is —C═N—CH₂— and Y¹ is CH, Y² is CH, Y³ is CH.

In another group of compounds of formula IE -B¹-B²-B³- is —C═N—CH₂—, Y¹ is CH, Y² is CH, Y³ is CH and R¹ is CF₃.

In another group of compounds of formula IE -B¹-B²-B³- is —C═N—CH₂—, Y¹ is CH, Y² is CH, Y³ is CH, R¹ is CF₃ and R⁹ is C₁-C₄alkyl.

In another group of compounds of formula IE -B¹-B²-B³- is —C═N—CH₂—, Y¹ is CH, Y² is CH, Y³ is CH, R¹ is CF₃, and R⁹ is C₁-C₄haloalkyl.

In another group of compounds of formula IE -B¹-B²-B³- is —C═N—CH₂—, Y¹ is CH, Y² is CH, Y³ is CH, R¹ is CF₃, R⁹ is C₃-C₄cycloalkyl.

In another group of compounds of formula IE -B¹-B²-B³- is —C═N—CH₂—, Y¹ is CH, Y² is CH, Y³ is CH, R¹ is CF₃, R⁹ is C₁-C₄alkyl, C₁-C₄haloalkyl or C₃-C₄cycloalkyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

In one group of compounds of formula IE -B¹-B²-B³- is —N—CH₂—CH₂—.

In another group of compounds of formula IE -B¹-B²-B³- is —N—CH₂—CH₂— and Y¹ is CH, Y² is CH, Y³ is CH.

In another group of compounds of formula IE -B¹-B²-B³- is —N—CH₂—CH₂—, Y¹ is CH, Y² is CH, Y³ is CH and R¹ is CF₃.

In another group of compounds of formula IE -B¹-B²-B³- is —N—CH₂—CH₂—, Y¹ is CH, Y² is CH, Y³ is CH, R¹ is CF₃ and R⁹ is C₁-C₄alkyl.

In another group of compounds of formula IE -B¹-B²-B³- is —N—CH₂—CH₂—, Y¹ is CH, Y² is CH, Y³ is CH, R¹ is CF₃, and R⁹ is C₁-C₄haloalkyl.

In another group of compounds of formula IE -B¹-B²-B³- is —N—CH₂—CH₂—, Y¹ is CH, Y² is CH, Y³ is CH, R¹ is CF₃, R⁹ is C₃-C₄cycloalkyl.

In another group of compounds of formula IE -B¹-B²-B³- is —N—CH₂—CH₂—, Y¹ is CH, Y² is CH, Y³ is CH, R¹ is CF₃, R⁹ is C₁-C₄alkyl, C₁-C₄haloalkyl or C₃-C₄cycloalkyl and R² is 3-chloro-5-bromophenyl-, 3-chloro-5-(trifluoromethyl)phenyl-, 3,5-dichloro-4-fluorophenyl-, 3,4,5-trichlorophenyl-, 3,5-bis(trifluoromethyl)phenyl-, 3-(trifluoromethyl)phenyl-, 2,6-dichloro-4-pyridyl-, 2,6-bis(trifluoromethyl)-4-pyridyl-, 3,5-dichloro-4-bromophenyl-, 3-bromo-5-(trifluoromethyl)phenyl-, 3,5-dibromophenyl-, or 3,4-dichlorophenyl-.

The following compounds of formula I-1, I-2 and I-3 illustrate the compounds of formula I when -B¹-B²-B³- is —C═N—O—, —C═N—CH₂—, or —N—CH₂—CH₂— respectively.

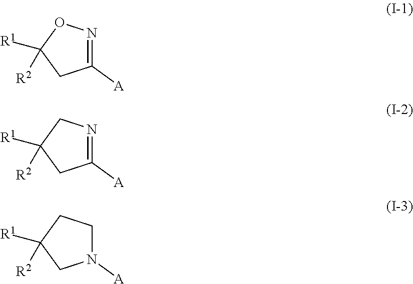

Tables 1 to 30 below illustrate compounds of the invention.

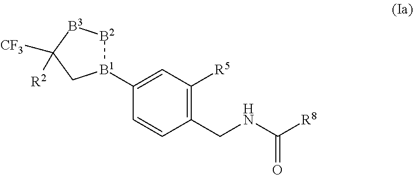

### Table 1

Table 1 provides 304 compounds of formula Ia wherein R5 is hydrogen, B1-B2-B3 is C═N—O, and R2 and R8 are as defined in Table P.

### Table 2

Table 2 provides 304 compounds of formula Ia wherein R5 is methyl, B1-B2-B3 is C═N—O, and R2 and R8 are as defined in Table P.

### Table 3

Table 3 provides 304 compounds of formula Ia wherein R5 is trifluoromethyl, B1-B2-B3 is C═N—O, and R2 and R8 are as defined in Table P.

### Table 4

Table 4 provides 304 compounds of formula Ia wherein R5 is chloro, B1-B2-B3 is C═N—O, and R2 and R8 are as defined in Table P.

### Table 5

Table 5 provides 304 compounds of formula Ia wherein R5 is bromo, B1-B2-B3 is C═N—O, and R2 and R8 are as defined in Table P.

### Table 6

Table 6 provides 304 compounds of formula Ia wherein R5 is hydrogen, B1-B2-B3 is C═N—CH2, and R2 and R8 are as defined in Table P.

### Table 7

Table 7 provides 304 compounds of formula Ia wherein R5 is methyl, B1-B2-B3 is C═N—CH2, and R2 and R8 are as defined in Table P.

### Table 8

Table 8 provides 304 compounds of formula Ia wherein R5 is trifluoromethyl, B1-B2-B3 is C═N—CH2, and R2 and R8 are as defined in Table P.

### Table 9

Table 9 provides 304 compounds of formula Ia wherein R5 is chloro, B1-B2-B3 is C═N—CH2, and R2 and R8 are as defined in Table P.

### Table 10

Table 10 provides 304 compounds of formula Ia wherein R5 is bromo, B1-B2-B3 is C═N—CH2, and R2 and R8 are as defined in Table P.

### Table 11

Table 11 provides 304 compounds of formula Ia wherein R5 is hydrogen, B1-B2-B3 is N—CH2-CH2, and R2 and R8 are as defined in Table P.

### Table 12

Table 12 provides 304 compounds of formula Ia wherein R5 is methyl, B1-B2-B3 is N—CH2-CH2, and R2 and R8 are as defined in Table P.

### Table 13

Table 13 provides 304 compounds of formula Ia wherein R5 is trifluoromethyl, B1-B2-B3 is N—CH2-CH2, and R2 and R8 are as defined in Table P.

### Table 14

Table 14 provides 304 compounds of formula Ia wherein R5 is chloro, B1-B2-B3 is N—CH2-CH2, and R2 and R8 are as defined in Table P.

### Table 15

Table 15 provides 304 compounds of formula Ia wherein R5 is bromo, B1-B2-B3 is N—CH2-CH2, and R2 and R8 are as defined in Table P.

TABLE P

| | R2 | R8 |
|---|---|---|
| 1 | 3,5-dichlorophenyl | methyl |
| 2 | 3-chloro-4-fluorophenyl | methyl |
| 3 | 3-fluoro-4-chlorophenyl | methyl |
| 4 | 3,4-dichlorophenyl | methyl |
| 5 | 3-chloro-4-bromophenyl | methyl |
| 6 | 3,5-dichloro-4-fluorophenyl | methyl |
| 7 | 3,4,5-trichlorophenyl | methyl |
| 8 | 3,5-dichloro-4-iodophenyl | methyl |
| 9 | 3,4,5-trifluorophenyl | methyl |
| 10 | 3-chloro-5-bromophenyl | methyl |
| 11 | 3-chloro-5-fluorophenyl | methyl |
| 12 | 3-chloro-5-(trifluoromethyl)phenyl | methyl |
| 13 | 3,4-dichloro-5-(trifluoromethyl)phenyl | methyl |
| 14 | 3,5-bis(trifluoromethyl)phenyl | methyl |
| 15 | 4-chloro-3,5-bis(trifluoromethyl)phenyl | methyl |
| 16 | 3-(trifluoromethyl)phenyl | methyl |
| 17 | 2,6-dichloro-4-pyridyl | methyl |
| 18 | 2,6-bis(trifluoromethyl)-4-pyridyl | methyl |
| 19 | 3-bromo-5-trifluoromethylphenyl | methyl |
| 20 | 3,5-dichlorophenyl | ethyl |
| 21 | 3-chloro-4-fluorophenyl | ethyl |
| 22 | 3-fluoro-4-chlorophenyl | ethyl |
| 23 | 3,4-dichlorophenyl | ethyl |
| 24 | 3-chloro-4-bromophenyl | ethyl |
| 25 | 3,5-dichloro-4-fluorophenyl | ethyl |
| 26 | 3,4,5-trichlorophenyl | ethyl |
| 27 | 3,5-dichloro-4-iodophenyl | ethyl |
| 28 | 3,4,5-trifluorophenyl | ethyl |
| 29 | 3-chloro-5-bromophenyl | ethyl |
| 30 | 3-chloro-5-fluorophenyl | ethyl |
| 31 | 3-chloro-5-(trifluoromethyl)phenyl | ethyl |
| 32 | 3,4-dichloro-5-(trifluoromethyl)phenyl | ethyl |
| 33 | 3,5-bis(trifluoromethyl)phenyl | ethyl |
| 34 | 4-chloro-3,5-bis(trifluoromethyl)phenyl | ethyl |
| 35 | 3-(trifluoromethyl)phenyl | ethyl |
| 36 | 2,6-dichloro-4-pyridyl | ethyl |
| 37 | 2,6-bis(trifluoromethyl)-4-pyridyl | ethyl |
| 38 | 3-bromo-5-trifluoromethylphenyl | ethyl |
| 39 | 3,5-dichlorophenyl | propyl |
| 40 | 3-chloro-4-fluorophenyl | propyl |
| 41 | 3-fluoro-4-chlorophenyl | propyl |
| 42 | 3,4-dichlorophenyl | propyl |
| 43 | 3-chloro-4-bromophenyl | propyl |
| 44 | 3,5-dichloro-4-fluorophenyl | propyl |
| 45 | 3,4,5-trichlorophenyl | propyl |
| 46 | 3,5-dichloro-4-iodophenyl | propyl |
| 47 | 3,4,5-trifluorophenyl | propyl |
| 48 | 3-chloro-5-bromophenyl | propyl |
| 49 | 3-chloro-5-fluorophenyl | propyl |
| 50 | 3-chloro-5-(trifluoromethyl)phenyl | propyl |
| 51 | 3,4-dichloro-5-(trifluoromethyl)phenyl | propyl |
| 52 | 3,5-bis(trifluoromethyl)phenyl | propyl |
| 53 | 4-chloro-3,5-bis(trifluoromethyl)phenyl | propyl |
| 54 | 3-(trifluoromethyl)phenyl | propyl |
| 55 | 2,6-dichloro-4-pyridyl | propyl |
| 56 | 2,6-bis(trifluoromethyl)-4-pyridyl | propyl |
| 57 | 3-bromo-5-trifluoromethylphenyl | propyl |
| 58 | 3,5-dichlorophenyl | isopropyl |
| 59 | 3-chloro-4-fluorophenyl | isopropyl |
| 60 | 3-fluoro-4-chlorophenyl | isopropyl |
| 61 | 3,4-dichlorophenyl | isopropyl |
| 62 | 3-chloro-4-bromophenyl | isopropyl |
| 63 | 3,5-dichloro-4-fluorophenyl | isopropyl |
| 64 | 3,4,5-trichlorophenyl | isopropyl |
| 65 | 3,5-dichloro-4-iodophenyl | isopropyl |
| 66 | 3,4,5-trifluorophenyl | isopropyl |
| 67 | 3-chloro-5-bromophenyl | isopropyl |
| 68 | 3-chloro-5-fluorophenyl | isopropyl |
| 69 | 3-chloro-5-(trifluoromethyl)phenyl | isopropyl |
| 70 | 3,4-dichloro-5-(trifluoromethyl)phenyl | isopropyl |
| 71 | 3,5-bis(trifluoromethyl)phenyl | isopropyl |
| 72 | 4-chloro-3,5-bis(trifluoromethyl)phenyl | isopropyl |
| 73 | 3-(trifluoromethyl)phenyl | isopropyl |
| 74 | 2,6-dichloro-4-pyridyl | isopropyl |
| 75 | 2,6-bis(trifluoromethyl)-4-pyridyl | isopropyl |
| 76 | 3-bromo-5-trifluoromethylphenyl | isopropyl |
| 77 | 3,5-dichlorophenyl | CH3CH2CH(CH3)— |
| 78 | 3-chloro-4-fluorophenyl | CH3CH2CH(CH3)— |
| 79 | 3-fluoro-4-chlorophenyl | CH3CH2CH(CH3)— |
| 80 | 3,4-dichlorophenyl | CH3CH2CH(CH3)— |
| 81 | 3-chloro-4-bromophenyl | CH3CH2CH(CH3)— |
| 82 | 3,5-dichloro-4-fluorophenyl | CH3CH2CH(CH3)— |
| 83 | 3,4,5-trichlorophenyl | CH3CH2CH(CH3)— |
| 84 | 3,5-dichloro-4-iodophenyl | CH3CH2CH(CH3)— |
| 85 | 3,4,5-trifluorophenyl | CH3CH2CH(CH3)— |
| 86 | 3-chloro-5-bromophenyl | CH3CH2CH(CH3)— |
| 87 | 3-chloro-5-fluorophenyl | CH3CH2CH(CH3)— |
| 88 | 3-chloro-5-(trifluoromethyl)phenyl | CH3CH2CH(CH3)— |
| 89 | 3,4-dichloro-5-(trifluoromethyl)phenyl | CH3CH2CH(CH3)— |
| 90 | 3,5-bis(trifluoromethyl)phenyl | CH3CH2CH(CH3)— |
| 91 | 4-chloro-3,5-bis(trifluoromethyl)phenyl | CH3CH2CH(CH3)— |
| 92 | 3-(trifluoromethyl)phenyl | CH3CH2CH(CH3)— |
| 93 | 2,6-dichloro-4-pyridyl | CH3CH2CH(CH3)— |
| 94 | 2,6-bis(trifluoromethyl)-4-pyridyl | CH3CH2CH(CH3)— |
| 95 | 3-bromo-5-trifluoromethylphenyl | CH3CH2CH(CH3)— |
| 96 | 3,5-dichlorophenyl | CH3OCH2— |
| 97 | 3-chloro-4-fluorophenyl | CH3OCH2— |
| 98 | 3-fluoro-4-chlorophenyl | CH3OCH2— |
| 99 | 3,4-dichlorophenyl | CH3OCH2— |
| 100 | 3-chloro-4-bromophenyl | CH3OCH2— |
| 101 | 3,5-dichloro-4-fluorophenyl | CH3OCH2— |
| 102 | 3,4,5-trichlorophenyl | CH3OCH2— |
| 103 | 3,5-dichloro-4-iodophenyl | CH3OCH2— |
| 104 | 3,4,5-trifluorophenyl | CH3OCH2— |
| 105 | 3-chloro-5-bromophenyl | CH3OCH2— |
| 106 | 3-chloro-5-fluorophenyl | CH3OCH2— |
| 107 | 3-chloro-5-(trifluoromethyl)phenyl | CH3OCH2— |
| 108 | 3,4-dichloro-5-(trifluoromethyl)phenyl | CH3OCH2— |
| 109 | 3,5-bis(trifluoromethyl)phenyl | CH3OCH2— |
| 110 | 4-chloro-3,5-bis(trifluoromethyl)phenyl | CH3OCH2— |
| 111 | 3-(trifluoromethyl)phenyl | CH3OCH2— |
| 112 | 2,6-dichloro-4-pyridyl | CH3OCH2— |
| 113 | 2,6-bis(trifluoromethyl)-4-pyridyl | CH3OCH2— |
| 114 | 3-bromo-5-trifluoromethylphenyl | CH3OCH2— |
| 115 | 3,5-dichlorophenyl | CH3OCH2CH2— |
| 116 | 3-chloro-4-fluorophenyl | CH3OCH2CH2— |
| 117 | 3-fluoro-4-chlorophenyl | CH3OCH2CH2— |
| 118 | 3,4-dichlorophenyl | CH3OCH2CH2— |
| 119 | 3-chloro-4-bromophenyl | CH3OCH2CH2— |
| 120 | 3,5-dichloro-4-fluorophenyl | CH3OCH2CH2— |
| 121 | 3,4,5-trichlorophenyl | CH3OCH2CH2— |
| 122 | 3,5-dichloro-4-iodophenyl | CH3OCH2CH2— |
| 123 | 3,4,5-trifluorophenyl | CH3OCH2CH2— |
| 124 | 3-chloro-5-bromophenyl | CH3OCH2CH2— |
| 125 | 3-chloro-5-fluorophenyl | CH3OCH2CH2— |
| 126 | 3-chloro-5-(trifluoromethyl)phenyl | CH3OCH2CH2— |
| 127 | 3,4-dichloro-5-(trifluoromethyl)phenyl | CH3OCH2CH2— |
| 128 | 3,5-bis(trifluoromethyl)phenyl | CH3OCH2CH2— |
| 129 | 4-chloro-3,5-bis(trifluoromethyl)phenyl | CH3OCH2CH2— |
| 130 | 3-(trifluoromethyl)phenyl | CH3OCH2CH2— |
| 131 | 2,6-dichloro-4-pyridyl | CH3OCH2CH2— |
| 132 | 2,6-bis(trifluoromethyl)-4-pyridyl | CH3OCH2CH2— |
| 133 | 3-bromo-5-trifluoromethylphenyl | CH3OCH2CH2— |
| 134 | 3,5-dichlorophenyl | CH3OCH(CH3)CH2— |
| 135 | 3-chloro-4-fluorophenyl | CH3OCH(CH3)CH2— |
| 136 | 3-fluoro-4-chlorophenyl | CH3OCH(CH3)CH2— |
| 137 | 3,4-dichlorophenyl | CH3OCH(CH3)CH2— |
| 138 | 3-chloro-4-bromophenyl | CH3OCH(CH3)CH2— |
| 139 | 3,5-dichloro-4-fluorophenyl | CH3OCH(CH3)CH2— |
| 140 | 3,4,5-trichlorophenyl | CH3OCH(CH3)CH2— |
| 141 | 3,5-dichloro-4-iodophenyl | CH3OCH(CH3)CH2— |
| 142 | 3,4,5-trifluorophenyl | CH3OCH(CH3)CH2— |
| 143 | 3-chloro-5-bromophenyl | CH3OCH(CH3)CH2— |
| 144 | 3-chloro-5-fluorophenyl | CH3OCH(CH3)CH2— |
| 145 | 3-chloro-5-(trifluoromethyl)phenyl | CH3OCH(CH3)CH2— |
| 146 | 3,4-dichloro-5-(trifluoromethyl)phenyl | CH3OCH(CH3)CH2— |
| 147 | 3,5-bis(trifluoromethyl)phenyl | CH3OCH(CH3)CH2— |
| 148 | 4-chloro-3,5-bis(trifluoromethyl)phenyl | CH3OCH(CH3)CH2— |
| 149 | 3-(trifluoromethyl)phenyl | CH3OCH(CH3)CH2— |
| 150 | 2,6-dichloro-4-pyridyl | CH3OCH(CH3)CH2— |
| 151 | 2,6-bis(trifluoromethyl)-4-pyridyl | CH3OCH(CH3)CH2— |
| 152 | 3-bromo-5-trifluoromethylphenyl | CH3OCH(CH3)CH2— |
| 153 | 3,5-dichlorophenyl | cyclopropyl |
| 154 | 3-chloro-4-fluorophenyl | cyclopropyl |
| 155 | 3-fluoro-4-chlorophenyl | cyclopropyl |
| 156 | 3,4-dichlorophenyl | cyclopropyl |
| 157 | 3-chloro-4-bromophenyl | cyclopropyl |
| 158 | 3,5-dichloro-4-fluorophenyl | cyclopropyl |
| 159 | 3,4,5-trichlorophenyl | cyclopropyl |
| 160 | 3,5-dichloro-4-iodophenyl | cyclopropyl |
| 161 | 3,4,5-trifluorophenyl | cyclopropyl |
| 162 | 3-chloro-5-bromophenyl | cyclopropyl |
| 163 | 3-chloro-5-fluorophenyl | cyclopropyl |
| 164 | 3-chloro-5-(trifluoromethyl)phenyl | cyclopropyl |
| 165 | 3,4-dichloro-5-(trifluoromethyl)phenyl | cyclopropyl |
| 166 | 3,5-bis(trifluoromethyl)phenyl | cyclopropyl |
| 167 | 4-chloro-3,5-bis(trifluoromethyl)phenyl | cyclopropyl |
| 168 | 3-(trifluoromethyl)phenyl | cyclopropyl |
| 169 | 2,6-dichloro-4-pyridyl | cyclopropyl |
| 170 | 2,6-bis(trifluoromethyl)-4-pyridyl | cyclopropyl |
| 171 | 3-bromo-5-trifluoromethylphenyl | cyclopropyl |
| 172 | 3,5-dichlorophenyl | Cyclopropyl-CH2— |
| 173 | 3-chloro-4-fluorophenyl | Cyclopropyl-CH2— |
| 174 | 3-fluoro-4-chlorophenyl | Cyclopropyl-CH2— |
| 175 | 3,4-dichlorophenyl | Cyclopropyl-CH2— |
| 176 | 3-chloro-4-bromophenyl | Cyclopropyl-CH2— |
| 177 | 3,5-dichloro-4-fluorophenyl | Cyclopropyl-CH2— |
| 178 | 3,4,5-trichlorophenyl | Cyclopropyl-CH2— |
| 179 | 3,5-dichloro-4-iodophenyl | Cyclopropyl-CH2— |
| 180 | 3,4,5-trifluorophenyl | Cyclopropyl-CH2— |
| 181 | 3-chloro-5-bromophenyl | Cyclopropyl-CH2— |
| 182 | 3-chloro-5-fluorophenyl | Cyclopropyl-CH2— |
| 183 | 3-chloro-5-(trifluoromethyl)phenyl | Cyclopropyl-CH2— |
| 184 | 3,4-dichloro-5-(trifluoromethyl)phenyl | Cyclopropyl-CH2— |
| 185 | 3,5-bis(trifluoromethyl)phenyl | Cyclopropyl-CH2— |
| 186 | 4-chloro-3,5-bis(trifluoromethyl)phenyl | Cyclopropyl-CH2— |
| 187 | 3-(trifluoromethyl)phenyl | Cyclopropyl-CH2— |
| 188 | 2,6-dichloro-4-pyridyl | Cyclopropyl-CH2— |
| 189 | 2,6-bis(trifluoromethyl)-4-pyridyl | Cyclopropyl-CH2— |
| 190 | 3-bromo-5-trifluoromethylphenyl | Cyclopropyl-CH2— |
| 191 | 3,5-dichlorophenyl | cyclobutyl |
| 192 | 3-chloro-4-fluorophenyl | cyclobutyl |
| 193 | 3-fluoro-4-chlorophenyl | cyclobutyl |
| 194 | 3,4-dichlorophenyl | cyclobutyl |
| 195 | 3-chloro-4-bromophenyl | cyclobutyl |
| 196 | 3,5-dichloro-4-fluorophenyl | cyclobutyl |
| 197 | 3,4,5-trichlorophenyl | cyclobutyl |
| 198 | 3,5-dichloro-4-iodophenyl | cyclobutyl |
| 199 | 3,4,5-trifluorophenyl | cyclobutyl |
| 200 | 3-chloro-5-bromophenyl | cyclobutyl |
| 201 | 3-chloro-5-fluorophenyl | cyclobutyl |
| 202 | 3-chloro-5-(trifluoromethyl)phenyl | cyclobutyl |
| 203 | 3,4-dichloro-5-(trifluoromethyl)phenyl | cyclobutyl |
| 204 | 3,5-bis(trifluoromethyl)phenyl | cyclobutyl |
| 205 | 4-chloro-3,5-bis(trifluoromethyl)phenyl | cyclobutyl |
| 206 | 3-(trifluoromethyl)phenyl | cyclobutyl |
| 207 | 2,6-dichloro-4-pyridyl | cyclobutyl |
| 208 | 2,6-bis(trifluoromethyl)-4-pyridyl | cyclobutyl |
| 209 | 3-bromo-5-trifluoromethylphenyl | cyclobutyl |
| 210 | 3,5-dichlorophenyl | CH3—S—CH2— |
| 211 | 3-chloro-4-fluorophenyl | CH3—S—CH2— |
| 212 | 3-fluoro-4-chlorophenyl | CH3—S—CH2— |
| 213 | 3,4-dichlorophenyl | CH3—S—CH2— |
| 214 | 3-chloro-4-bromophenyl | CH3—S—CH2— |
| 215 | 3,5-dichloro-4-fluorophenyl | CH3—S—CH2— |
| 216 | 3,4,5-trichlorophenyl | CH3—S—CH2— |
| 217 | 3,5-dichloro-4-iodophenyl | CH3—S—CH2— |
| 218 | 3,4,5-trifluorophenyl | CH3—S—CH2— |
| 219 | 3-chloro-5-bromophenyl | CH3—S—CH2— |
| 220 | 3-chloro-5-fluorophenyl | CH3—S—CH2— |
| 221 | 3-chloro-5-(trifluoromethyl)phenyl | CH3—S—CH2— |
| 222 | 3,4-dichloro-5-(trifluoromethyl)phenyl | CH3—S—CH2— |
| 223 | 3,5-bis(trifluoromethyl)phenyl | CH3—S—CH2— |
| 224 | 4-chloro-3,5-bis(trifluoromethyl)phenyl | CH3—S—CH2— |
| 225 | 3-(trifluoromethyl)phenyl | CH3—S—CH2— |
| 226 | 2,6-dichloro-4-pyridyl | CH3—S—CH2— |
| 227 | 2,6-bis(trifluoromethyl)-4-pyridyl | CH3—S—CH2— |
| 228 | 3-bromo-5-trifluoromethylphenyl | CH3—S—CH2— |
| 229 | 3,5-dichlorophenyl | CH3—S(O)—CH2— |
| 230 | 3-chloro-4-fluorophenyl | CH3—S(O)—CH2— |
| 231 | 3-fluoro-4-chlorophenyl | CH3—S(O)—CH2— |
| 232 | 3,4-dichlorophenyl | CH3—S(O)—CH2— |
| 233 | 3-chloro-4-bromophenyl | CH3—S(O)—CH2— |
| 234 | 3,5-dichloro-4-fluorophenyl | CH3—S(O)—CH2— |
| 235 | 3,4,5-trichlorophenyl | CH3—S(O)—CH2— |
| 236 | 3,5-dichloro-4-iodophenyl | CH3—S(O)—CH2— |
| 237 | 3,4,5-trifluorophenyl | CH3—S(O)—CH2— |
| 238 | 3-chloro-5-bromophenyl | CH3—S(O)—CH2— |
| 239 | 3-chloro-5-fluorophenyl | CH3—S(O)—CH2— |
| 240 | 3-chloro-5-(trifluoromethyl)phenyl | CH3—S(O)—CH2— |
| 241 | 3,4-dichloro-5-(trifluoromethyl)phenyl | CH3—S(O)—CH2— |
| 242 | 3,5-bis(trifluoromethyl)phenyl | CH3—S(O)—CH2— |
| 243 | 4-chloro-3,5-bis(trifluoromethyl)phenyl | CH3—S(O)—CH2— |
| 244 | 3-(trifluoromethyl)phenyl | CH3—S(O)—CH2— |
| 245 | 2,6-dichloro-4-pyridyl | CH3—S(O)—CH2— |
| 246 | 2,6-bis(trifluoromethyl)-4-pyridyl | CH3—S(O)—CH2— |
| 247 | 3-bromo-5-trifluoromethylphenyl | CH3—S(O)—CH2— |
| 248 | 3,5-dichlorophenyl | CH3—S(O2)—CH2— |
| 249 | 3-chloro-4-fluorophenyl | CH3—S(O2)—CH2— |
| 250 | 3-fluoro-4-chlorophenyl | CH3—S(O2)—CH2— |
| 251 | 3,4-dichlorophenyl | CH3—S(O2)—CH2— |
| 252 | 3-chloro-4-bromophenyl | CH3—S(O2)—CH2— |
| 253 | 3,5-dichloro-4-fluorophenyl | CH3—S(O2)—CH2— |
| 254 | 3,4,5-trichlorophenyl | CH3—S(O2)—CH2— |
| 255 | 3,5-dichloro-4-iodophenyl | CH3—S(O2)—CH2— |
| 256 | 3,4,5-trifluorophenyl | CH3—S(O2)—CH2— |
| 257 | 3-chloro-5-bromophenyl | CH3—S(O2)—CH2— |
| 258 | 3-chloro-5-fluorophenyl | CH3—S(O2)—CH2— |
| 259 | 3-chloro-5-(trifluoromethyl)phenyl | CH3—S(O2)—CH2— |
| 260 | 3,4-dichloro-5-(trifluoromethyl)phenyl | CH3—S(O2)—CH2— |
| 261 | 3,5-bis(trifluoromethyl)phenyl | CH3—S(O2)—CH2— |
| 262 | 4-chloro-3,5-bis(trifluoromethyl)phenyl | CH3—S(O2)—CH2— |
| 263 | 3-(trifluoromethyl)phenyl | CH3—S(O2)—CH2— |
| 264 | 2,6-dichloro-4-pyridyl | CH3—S(O2)—CH2— |
| 265 | 2,6-bis(trifluoromethyl)-4-pyridyl | CH3—S(O2)—CH2— |
| 266 | 3-bromo-5-trifluoromethylphenyl | CH3—S(O2)—CH2— |
| 267 | 3,5-dichlorophenyl | tetrahydrofuran-2-yl |
| 268 | 3-chloro-4-fluorophenyl | tetrahydrofuran-2-yl |
| 269 | 3-fluoro-4-chlorophenyl | tetrahydrofuran-2-yl |
| 270 | 3,4-dichlorophenyl | tetrahydrofuran-2-yl |
| 271 | 3-chloro-4-bromophenyl | tetrahydrofuran-2-yl |
| 272 | 3,5-dichloro-4-fluorophenyl | tetrahydrofuran-2-yl |
| 273 | 3,4,5-trichlorophenyl | tetrahydrofuran-2-yl |
| 274 | 3,5-dichloro-4-iodophenyl | tetrahydrofuran-2-yl |
| 275 | 3,4,5-trifluorophenyl | tetrahydrofuran-2-yl |
| 276 | 3-chloro-5-bromophenyl | tetrahydrofuran-2-yl |
| 277 | 3-chloro-5-fluorophenyl | tetrahydrofuran-2-yl |
| 278 | 3-chloro-5-(trifluoromethyl)phenyl | tetrahydrofuran-2-yl |
| 279 | 3,4-dichloro-5-(trifluoromethyl)phenyl | tetrahydrofuran-2-yl |
| 280 | 3,5-bis(trifluoromethyl)phenyl | tetrahydrofuran-2-yl |
| 281 | 4-chloro-3,5-bis(trifluoromethyl)phenyl | tetrahydrofuran-2-yl |
| 282 | 3-(trifluoromethyl)phenyl | tetrahydrofuran-2-yl |
| 283 | 2,6-dichloro-4-pyridyl | tetrahydrofuran-2-yl |
| 284 | 2,6-bis(trifluoromethyl)-4-pyridyl | tetrahydrofuran-2-yl |
| 285 | 3-bromo-5-trifluoromethylphenyl | tetrahydrofuran-2-yl |
| 286 | 3,5-dichlorophenyl | tetrahydrofuran-3-yl |
| 287 | 3-chloro-4-fluorophenyl | tetrahydrofuran-3-yl |
| 288 | 3-fluoro-4-chlorophenyl | tetrahydrofuran-3-yl |
| 289 | 3,4-dichlorophenyl | tetrahydrofuran-3-yl |
| 290 | 3-chloro-4-bromophenyl | tetrahydrofuran-3-yl |
| 291 | 3,5-dichloro-4-fluorophenyl | tetrahydrofuran-3-yl |
| 292 | 3,4,5-trichlorophenyl | tetrahydrofuran-3-yl |
| 293 | 3,5-dichloro-4-iodophenyl | tetrahydrofuran-3-yl |
| 294 | 3,4,5-trifluorophenyl | tetrahydrofuran-3-yl |
| 295 | 3-chloro-5-bromophenyl | tetrahydrofuran-3-yl |
| 296 | 3-chloro-5-fluorophenyl | tetrahydrofuran-3-yl |
| 297 | 3-chloro-5-(trifluoromethyl)phenyl | tetrahydrofuran-3-yl |
| 298 | 3,4-dichloro-5-(trifluoromethyl)phenyl | tetrahydrofuran-3-yl |
| 299 | 3,5-bis(trifluoromethyl)phenyl | tetrahydrofuran-3-yl |
| 300 | 4-chloro-3,5-bis(trifluoromethyl)phenyl | tetrahydrofuran-3-yl |
| 301 | 3-(trifluoromethyl)phenyl | tetrahydrofuran-3-yl |
| 302 | 2,6-dichloro-4-pyridyl | tetrahydrofuran-3-yl |
| 303 | 2,6-bis(trifluoromethyl)-4-pyridyl | tetrahydrofuran-3-yl |
| 304 | 3-bromo-5-trifluoromethylphenyl | tetrahydrofuran-3-yl |

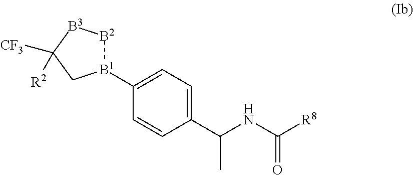

### Table 16

Table 16 provides 304 compounds of formula Ib wherein B1-B2-B3 is C═N—O, and R2 and R8 are as defined in Table P.

### Table 17

Table 17 provides 304 compounds of formula Ib wherein B1-B2-B3 is C═N—CH2, and R2 and R8 are as defined in Table P.

### Table 18

Table 18 provides 304 compounds of formula Ib wherein B1-B2-B3 is N—CH2-CH2, and R2 and R8 are as defined in Table P.

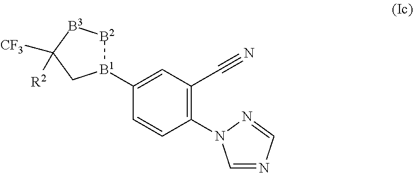

### Table 19

Table 19 provides 19 compounds of formula Ic wherein B1-B2-B3 is C═N—O, and R2 is as defined in Table Q.

### Table 20

Table 20 provides 19 compounds of formula Ic wherein B1-B2-B3 is C═N—CH2, and R2 is as defined in Table Q.

### Table 21

Table 21 provides 19 compounds of formula Ic wherein B1-B2-B3 is N—CH2-CH2, and R2 is as defined in Table Q.

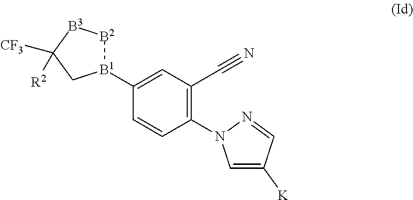

### Table 22

Table 22 provides 19 compounds of formula Id wherein k is CN, B1-B2-B3 is C═N—O and R2 is as defined in Table Q.

### Table 23

Table 23 provides 19 compounds of formula Id wherein k is CF3, B1-B2-B3 is C═N—O and R2 is as defined in Table Q.

### Table 24

Table 24 provides 19 compounds of formula Id wherein k is CN, B1-B2-B3 is C═N—CH2 and R2 is as defined in Table Q.

### Table 25

Table 25 provides 19 compounds of formula Id wherein k is CF3, B1-B2-B3 is C═N—CH2 and R2 is as defined in Table Q.

### Table 26

Table 26 provides 19 compounds of formula Id wherein k is CN, B1-B2-B3 is N—CH2-CH2 and R2 is as defined in Table Q.

### Table 27

Table 27 provides 19 compounds of formula Id wherein k is CF3, B1-B2-B3 is N—CH2-CH2 and R2 is as defined in Table Q.

TABLE Q

| | R2 |
|---|---|
| 1 | 3,5-dichlorophenyl |
| 2 | 3-chloro-4-fluorophenyl |
| 3 | 3-fluoro-4-chlorophenyl |
| 4 | 3,4-dichlorophenyl |
| 5 | 3-chloro-4-bromophenyl |
| 6 | 3,5-dichloro-4-fluorophenyl |
| 7 | 3,4,5-trichlorophenyl |
| 8 | 3,5-dichloro-4-iodophenyl |
| 9 | 3,4,5-trifluorophenyl |
| 10 | 3-chloro-5-bromophenyl |
| 11 | 3-chloro-5-fluorophenyl |
| 12 | 3-chloro-5-(trifluoromethyl)phenyl |
| 13 | 3,4-dichloro-5-(trifluoromethyl)phenyl |
| 14 | 3,5-bis(trifluoromethyl)phenyl |
| 15 | 4-chloro-3,5-bis(trifluoromethyl)phenyl |
| 16 | 3-(trifluoromethyl)phenyl |
| 17 | 2,6-dichloro-4-pyridyl |
| 18 | 2,6-bis(trifluoromethyl)-4-pyridyl |
| 19 | 3-bromo-5-trifluoromethylphenyl |

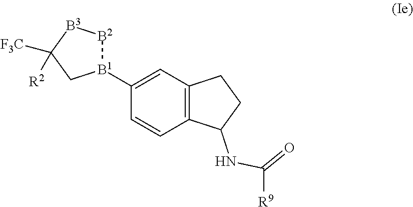

### Table 28

Table 28 provides 114 compounds of formula Ie wherein B1-B2-B3 is C═N—O, and R2 and R9 are as defined in Table S.

### Table 29

Table 29 provides 114 compounds of formula Ie wherein B1-B2-B3 is C═N—CH2, and R2 and R9 are as defined in Table S.

### Table 30

Table 30 provides 114 compounds of formula Ie wherein B1-B2-B3 is N—CH2-CH2, and R2 and R9 are as defined in Table S.

TABLE S

| | R2 | R9 |
|---|---|---|
| 1 | 3,5-dichlorophenyl | methyl |
| 2 | 3-chloro-4-fluorophenyl | methyl |
| 3 | 3-fluoro-4-chlorophenyl | methyl |
| 4 | 3,4-dichlorophenyl | methyl |
| 5 | 3-chloro-4-bromophenyl | methyl |
| 6 | 3,5-dichloro-4-fluorophenyl | methyl |
| 7 | 3,4,5-trichlorophenyl | methyl |
| 8 | 3,5-dichloro-4-iodophenyl | methyl |
| 9 | 3,4,5-trifluorophenyl | methyl |
| 10 | 3-chloro-5-bromophenyl | methyl |
| 11 | 3-chloro-5-fluorophenyl | methyl |
| 12 | 3-chloro-5-(trifluoromethyl)phenyl | methyl |
| 13 | 3,4-dichloro-5-(trifluoromethyl)phenyl | methyl |
| 14 | 3,5-bis(trifluoromethyl)phenyl | methyl |
| 15 | 4-chloro-3,5-bis(trifluoromethyl)phenyl | methyl |
| 16 | 3-(trifluoromethyl)phenyl | methyl |
| 17 | 2,6-dichloro-4-pyridyl | methyl |
| 18 | 2,6-bis(trifluoromethyl)-4-pyridyl | methyl |
| 19 | 3-bromo-5-trifluoromethylphenyl | methyl |
| 20 | 3,5-dichlorophenyl | ethyl |
| 21 | 3-chloro-4-fluorophenyl | ethyl |
| 22 | 3-fluoro-4-chlorophenyl | ethyl |
| 23 | 3,4-dichlorophenyl | ethyl |
| 24 | 3-chloro-4-bromophenyl | ethyl |
| 25 | 3,5-dichloro-4-fluorophenyl | ethyl |
| 26 | 3,4,5-trichlorophenyl | ethyl |
| 27 | 3,5-dichloro-4-iodophenyl | ethyl |
| 28 | 3,4,5-trifluorophenyl | ethyl |
| 29 | 3-chloro-5-bromophenyl | ethyl |
| 30 | 3-chloro-5-fluorophenyl | ethyl |
| 31 | 3-chloro-5-(trifluoromethyl)phenyl | ethyl |
| 32 | 3,4-dichloro-5-(trifluoromethyl)phenyl | ethyl |
| 33 | 3,5-bis(trifluoromethyl)phenyl | ethyl |
| 34 | 4-chloro-3,5-bis(trifluoromethyl)phenyl | ethyl |
| 35 | 3-(trifluoromethyl)phenyl | ethyl |
| 36 | 2,6-dichloro-4-pyridyl | ethyl |
| 37 | 2,6-bis(trifluoromethyl)-4-pyridyl | ethyl |
| 38 | 3-bromo-5-trifluoromethylphenyl | ethyl |
| 39 | 3,5-dichlorophenyl | cyclopropyl |
| 40 | 3-chloro-4-fluorophenyl | cyclopropyl |
| 41 | 3-fluoro-4-chlorophenyl | cyclopropyl |
| 42 | 3,4-dichlorophenyl | cyclopropyl |
| 43 | 3-chloro-4-bromophenyl | cyclopropyl |
| 44 | 3,5-dichloro-4-fluorophenyl | cyclopropyl |
| 45 | 3,4,5-trichlorophenyl | cyclopropyl |
| 46 | 3,5-dichloro-4-iodophenyl | cyclopropyl |
| 47 | 3,4,5-trifluorophenyl | cyclopropyl |
| 48 | 3-chloro-5-bromophenyl | cyclopropyl |
| 49 | 3-chloro-5-fluorophenyl | cyclopropyl |
| 50 | 3-chloro-5-(trifluoromethyl)phenyl | cyclopropyl |
| 51 | 3,4-dichloro-5-(trifluoromethyl)phenyl | cyclopropyl |
| 52 | 3,5-bis(trifluoromethyl)phenyl | cyclopropyl |
| 53 | 4-chloro-3,5-bis(trifluoromethyl)phenyl | cyclopropyl |
| 54 | 3-(trifluoromethyl)phenyl | cyclopropyl |
| 55 | 2,6-dichloro-4-pyridyl | cyclopropyl |
| 56 | 2,6-bis(trifluoromethyl)-4-pyridyl | cyclopropyl |
| 57 | 3-bromo-5-trifluoromethylphenyl | cyclopropyl |
| 58 | 3,5-dichlorophenyl | CF3CH2— |
| 59 | 3-chloro-4-fluorophenyl | CF3CH2— |
| 60 | 3-fluoro-4-chlorophenyl | CF3CH2— |
| 61 | 3,4-dichlorophenyl | CF3CH2— |
| 62 | 3-chloro-4-bromophenyl | CF3CH2— |
| 63 | 3,5-dichloro-4-fluorophenyl | CF3CH2— |
| 64 | 3,4,5-trichlorophenyl | CF3CH2— |
| 65 | 3,5-dichloro-4-iodophenyl | CF3CH2— |
| 66 | 3,4,5-trifluorophenyl | CF3CH2— |
| 67 | 3-chloro-5-bromophenyl | CF3CH2— |
| 68 | 3-chloro-5-fluorophenyl | CF3CH2— |
| 69 | 3-chloro-5-(trifluoromethyl)phenyl | CF3CH2— |
| 70 | 3,4-dichloro-5-(trifluoromethyl)phenyl | CF3CH2— |
| 71 | 3,5-bis(trifluoromethyl)phenyl | CF3CH2— |
| 72 | 4-chloro-3,5-bis(trifluoromethyl)phenyl | CF3CH2— |
| 73 | 3-(trifluoromethyl)phenyl | CF3CH2— |
| 74 | 2,6-dichloro-4-pyridyl | CF3CH2— |
| 75 | 2,6-bis(trifluoromethyl)-4-pyridyl | CF3CH2— |
| 76 | 3-bromo-5-trifluoromethylphenyl | CF3CH2— |
| 77 | 3,5-dichlorophenyl | cyclopropyl-CH2— |
| 78 | 3-chloro-4-fluorophenyl | cyclopropyl-CH2— |
| 79 | 3-fluoro-4-chlorophenyl | cyclopropyl-CH2— |
| 80 | 3,4-dichlorophenyl | cyclopropyl-CH2— |
| 81 | 3-chloro-4-bromophenyl | cyclopropyl-CH2— |
| 82 | 3,5-dichloro-4-fluorophenyl | cyclopropyl-CH2— |
| 83 | 3,4,5-trichlorophenyl | cyclopropyl-CH2— |
| 84 | 3,5-dichloro-4-iodophenyl | cyclopropyl-CH2— |
| 85 | 3,4,5-trifluorophenyl | cyclopropyl-CH2— |
| 86 | 3-chloro-5-bromophenyl | cyclopropyl-CH2— |
| 87 | 3-chloro-5-fluorophenyl | cyclopropyl-CH2— |
| 88 | 3-chloro-5-(trifluoromethyl)phenyl | cyclopropyl-CH2— |
| 89 | 3,4-dichloro-5-(trifluoromethyl)phenyl | cyclopropyl-CH2— |
| 90 | 3,5-bis(trifluoromethyl)phenyl | cyclopropyl-CH2— |
| 91 | 4-chloro-3,5-bis(trifluoromethyl)phenyl | cyclopropyl-CH2— |
| 92 | 3-(trifluoromethyl)phenyl | cyclopropyl-CH2— |
| 93 | 2,6-dichloro-4-pyridyl | cyclopropyl-CH2— |
| 94 | 2,6-bis(trifluoromethyl)-4-pyridyl | cyclopropyl-CH2— |
| 95 | 3-bromo-5-trifluoromethylphenyl | cyclopropyl-CH2— |
| 96 | 3,5-dichlorophenyl | isopropyl |
| 97 | 3-chloro-4-fluorophenyl | isopropyl |
| 98 | 3-fluoro-4-chlorophenyl | isopropyl |
| 99 | 3,4-dichlorophenyl | isopropyl |
| 100 | 3-chloro-4-bromophenyl | isopropyl |
| 101 | 3,5-dichloro-4-fluorophenyl | isopropyl |
| 102 | 3,4,5-trichlorophenyl | isopropyl |
| 103 | 3,5-dichloro-4-iodophenyl | isopropyl |
| 104 | 3,4,5-trifluorophenyl | isopropyl |
| 105 | 3-chloro-5-bromophenyl | isopropyl |
| 106 | 3-chloro-5-fluorophenyl | isopropyl |
| 107 | 3-chloro-5-(trifluoromethyl)phenyl | isopropyl |
| 108 | 3,4-dichloro-5-(trifluoromethyl)phenyl | isopropyl |
| 109 | 3,5-bis(trifluoromethyl)phenyl | isopropyl |
| 110 | 4-chloro-3,5-bis(trifluoromethyl)phenyl | isopropyl |
| 111 | 3-(trifluoromethyl)phenyl | isopropyl |
| 112 | 2,6-dichloro-4-pyridyl | isopropyl |
| 113 | 2,6-bis(trifluoromethyl)-4-pyridyl | isopropyl |
| 114 | 3-bromo-5-trifluoromethylphenyl | isopropyl |

Compounds of formula I include at least one chiral centre and may exist as compounds of formula I* or compounds of formula I**. Compounds I* and I** are enantiomers if there is no other chiral center or epimers otherwise.

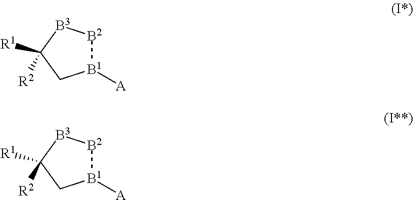

Generally compounds of formula I** are more biologically active than compounds of formula I*. The invention includes mixtures of compounds I* and I** in any ratio e.g. in a molar ratio of 1:99 to 99:1, e.g. 10:1 to 1:10, e.g. a substantially 50:50 molar ratio. In an enantiomerically (or epimerically) enriched mixture of formula I**, the molar proportion of compound I** compared to the total amount of both enantiomers (or epimers) is for example greater than 50%, e.g. at least 55, 60, 65, 70, 75, 80, 85, 90, 95, 96, 97, 98, or at least 99%. Likewise, in enantiomerically (or epimerically) enriched mixtures of formula I*, the molar proportion of the compound of formula I* compared to the total amount of both enantiomers (or epimers) is for example greater than 50%, e.g. at least 55, 60, 65, 70, 75, 80, 85, 90, 95, 96, 97, 98, or at least 99%. Enantiomerically (or epimerically) enriched mixtures of formula I** are preferred. Each compound disclosed in Tables 1 to 30 represents a disclosure of a compound according to the compound of formula I* and a compound according to the compound of formula I**.

Likewise, group A2 may be group A2* or A2**.

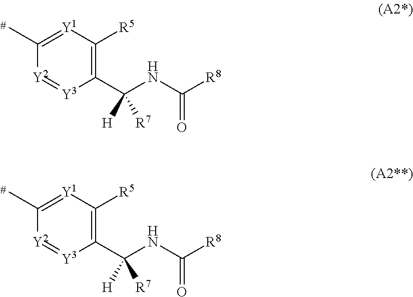

The invention includes mixtures of compounds of formula I with A2 as A2* and A2** in any ratio e.g. in a molar ratio of 1:99 to 99:1, e.g. 10:1 to 1:10, e.g. a substantially 50:50 molar ratio. In an enantiomerically (or epimerically) enriched mixture of formula I with A2 as A2*, the molar proportion of formula I with A2 as A2* compared to the total amount of both enantiomers (or epimers) is for example greater than 50%, e.g. at least 55, 60, 65, 70, 75, 80, 85, 90, 95, 96, 97, 98, or at least 99%. Likewise, in enantiomerically (or epimerically) enriched mixture of formula I with A2 as A2**, the molar proportion of the compound of formula I with A2 as A2**, compared to the total amount of both enantiomers (or epimers) is for example greater than 50%, e.g. at least 55, 60, 65, 70, 75, 80, 85, 90, 95, 96, 97, 98, or at least 99%. Enantiomerically (or epimerically) enriched mixtures of formula I with A2 as A2** are preferred.

Each compound disclosed in Tables 16 to 18 represents a disclosure of I with A2 as A2* and a compound I with A2 as A2**. Each compound disclosed in Tables 16 to 18 represents a disclosure of a compound according to formula I* with A2 as A2*. Each compound disclosed in Tables 16 to 18 represents a disclosure of a compound according to formula I** with A2 as A2*. Each compound disclosed in Tables 16 to 18 represents a disclosure of a compound according to formula I* with A2 as A2**. Each compound disclosed in Tables 16 to 18 represents a disclosure of a compound according to formula I** with A2 as A2**. Enantiomerically (or epimerically) enriched mixtures of formula I** with A2 as A2** are preferred.

Likewise, group A5 may be A5* or A5**

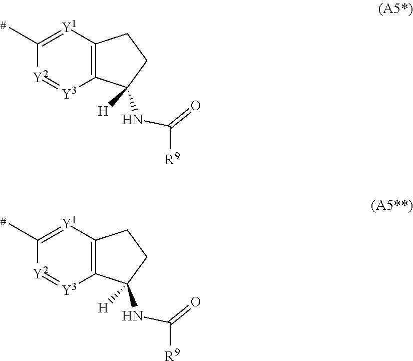

The invention includes mixtures of compounds of formula I with A5 as A5* and A5** in any ratio e.g. in a molar ratio of 1:99 to 99:1, e.g. 10:1 to 1:10, e.g. a substantially 50:50 molar ratio. In an enantiomerically (or epimerically) enriched mixture of formula I with A5 as A5*, the molar proportion of formula I with A5 as A5* compared to the total amount of both enantiomers (or epimers) is for example greater than 50%, e.g. at least 55, 60, 65, 70, 75, 80, 85, 90, 95, 96, 97, 98, or at least 99%. Likewise, in enantiomerically (or epimerically) enriched mixture of formula I with A5 as A5**, the molar proportion of the compound of formula I with A5 as A5**, compared to the total amount of both enantiomers (or epimers) is for example greater than 50%, e.g. at least 55, 60, 65, 70, 75, 80, 85, 90, 95, 96, 97, 98, or at least 99%.

Compounds of formula A5* are preferred. Each compound disclosed in Tables 28 to 30 represents a disclosure of I with A5 as A5* and a compound I with A5 as A5**. Each compound disclosed in Tables 28 to 30 represents a disclosure of a compound according to formula I* with A5 as A5*. Each compound disclosed in Tables 28 to 30 represents a disclosure of a compound according to formula I** with A5 as A5*. Each compound disclosed in Tables 28 to 30 represents a disclosure of a compound according to formula I* with A5 as A5**. Each compound disclosed in Tables 28 to 30 represents a disclosure of a compound according to formula I** with A5 as A5**.

The compounds of formula I may be prepared as described in WO 2008/128711, WO 2010/043315, The compounds of formula I may be prepared as described in WO 2008/128711, WO 2010/043315, WO 2011/051455, WO 2007/105814, WO 2008/122375, WO 2009/035004, WO 2009/045999, WO 2009/072621, WO 2009/097992, WO 2010/133336, WO 2010/043315, WO 2011/051455, WO 2011/080211, JP2010235590, JP2011037817, JP2011178724, CN102210317, CN102246777, WO 2009/07261, WO 2009/097992, WO 2009/051956, each of which is incorporated herein by reference.

In one embodiment the invention provides a compound selected from Tables 1 to 15 for use against insects of the family Curculionidae, preferably in for use against Anthonomus grandis.

In one embodiment the invention provides a compound selected from Tables 16 to 18 for use against insects of the family Curculionidae, preferably in for use against Anthonomus grandis.

In one embodiment the invention provides a compound selected from Tables 19 to 21 for use against insects of the family Curculionidae, preferably in for use against Anthonomus grandis.

In one embodiment the invention provides a compound selected from Tables 22 to 27 for use against insects of the family Curculionidae, preferably in for use against Anthonomus grandis.

In one embodiment the invention provides a compound selected from Tables 28 to 30 for use against insects of the family Curculionidae, preferably in for use against Anthonomus grandis.

In one embodiment the invention provides a compound selected from Tables 1 to 15 for use against Anthonomus grandis in cotton.

In one embodiment the invention provides a compound selected from Tables 16 to 18 for use against Anthonomus grandis in cotton.

In one embodiment the invention provides a compound selected from Tables 19 to 21 for use against Anthonomus grandis in cotton.

In one embodiment the invention provides a compound selected from Tables 22 to 27 for use against Anthonomus grandis in cotton.

In one embodiment the invention provides a compound selected from Tables 28 to 30 for use against Anthonomus grandis in cotton.

Examples of insects from the family of Curculionidae. are Anthonomus corvulus, Anthonomus elutus, Anthonomus elongatus, Anthonomus eugenii, Anthonomus consors, Anthonomus haematopus, Anthonomus lecontei, Anthonomus molochinus, Anthonomus morticinus, Anthonomus musculus, Anthonomus nigrinus, Anthonomus phyllocola, Anthonomus pictus, Anthonomus pomorum, Anthonomus quadrigibbus, Anthonomus rectirostris, Anthonomus rubi, Anthonomus santacruzi, Anthonomus signatus, Anthonomus subfasciatus, and Anthonomus tenebrosus. Preferably the insects from the family of Curculionidae are of the genus Anthonomus, preferably of the species Anthonomus grandis, Conotrachelus nenuphar, Ceutorhynchus spp., or Curculio nucum, more preferably of the species Anthonomus grandis.

In one embodiment the methods and uses of the invention are for controlling and/or preventing infestation of a cotton crop by Anthonomus grandis (commonly known as boll weevil) including Anthonomus grandis that is resistant to other insecticides, e.g. pyrethroid insecticides. Anthonomus grandis that is “resistant” to a particular insecticide refers e.g. to strains of Anthonomus grandis that are less sensitive to that insecticide compared to the expected sensitivity of the same species of Anthonomus grandis. The expected sensitivity can be measured using e.g. a strain that has not previously been exposed to the insecticide.

In another embodiment the methods In one embodiment the methods and uses of the invention are for controlling lepidopteran leaf feeders, bollworms, bollweevil, aphids, whitefly, thrips, lygus, mites, cutworms, soil insects, and/or nematodes.

In another embodiment the compounds of the invention may be used on cotton to control, for example, Anthonomus grandis, Pectinophora spp., heliothis spp., Spodoptera spp., Tetranychus spp., Empoasca spp., Thrips spp., Bemisia tabaci, Trialeurodes spp., Aphids, Lygus spp., phyllophaga spp., Scaptocoris spp., Austroasca viridigrisea, Creontiades spp., Nezara spp., Piezodorus spp., Halotydeus destructor, Oxycaraenus hyalinipennis, Dysdercus cingulatus. In particular, the compounds of the invention are used on cotton to control Anthonomus grandis, Tetranychus spp., Empoasca spp., thrips spp., Lygus spp., phyllophaga spp., Scaptocoris spp.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of a plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Application may be before infestation or when the pest is present. Application of the compounds of the invention can be performed according to any of the usual modes of application, e.g. foliar, drench, soil, in furrow etc. However, control of Anthonomus grandis s is usually achieved by foliar application, which is the preferred mode of application according to the invention.

Application of the compounds of the invention is preferably to a crop of cotton plants, the locus thereof or propagation material thereof

The compounds of the invention may be applied to plant parts. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds. Treatment according to the invention of the plants and plant parts with the active compounds can be carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

The compounds of the invention are suitable for use on any plant (preferably cotton plant), including those that have been genetically modified to be resistant to active ingredients such as herbicides, or to produce biologically active compounds that control infestation by plant pests.

The term “plant” as used herein includes seedlings, bushes and trees. Crops are to be understood as also including those crops which have been rendered tolerant to herbicides or classes of herbicides (e.g. ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Compounds of formula I may be used on transgenic plants (including cultivars) obtained by genetic engineering methods and/or by conventional methods. These are understood as meaning plants having novel properties (“traits”) which have been obtained by conventional breeding, by mutagenesis or by recombinant DNA techniques. Depending on the plant species or plant cultivars, their location and growth conditions (soils, climate, vegetation period, diet), the treatment according to the invention may also result in superadditive “synergistic”) effects.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products.

Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds.

Examples of transgenic plants which may be mentioned are the important crop plants, such as cereals (wheat, rice), maize, soybean, potatoes, sugar beet, tomatoes, peas and other vegetable varieties, cotton, tobacco, oilseed rape and also fruit plants (with the fruits apples, pears, citrus fruits and grapes). Transgenic cotton is of particular interest.

Compounds of formula I may be used on transgenic plants that are capable of producing one or more pesticidal proteins which confer upon the transgenic plant tolerance or resistance to harmful pests, e.g. insect pests, nematode pests and the like. Such pesticidal proteins include, without limitation, Cry proteins from Bacillus thuringiensis Cry1Ab, Cry1Ac, Cry1F, Cry2Ab, Cry2Ae, Cry3A, Cry3Bb, or Cry9C; engineered proteins such as modified Cry3A (U.S. Pat. No. 7,030,295) or Cry1A.105; or vegetative insecticidal proteins such as Vip1, Vip2 or Vip3. A full list of Bt Cry proteins and VIPs useful in the invention can be found on the worldwide web at Bacillus thuringiensis Toxin Nomenclature Database maintained by the University of Sussex (see also, Crickmore et al. (1998) Microbiol. Mol. Biol. Rev. 62:807-813). Other pesticidal proteins useful in the invention include proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. Further examples of such pesticidal proteins or transgenic plants capable of synthesizing such proteins are disclosed, e.g., in EP-A 374753, WO 93/007278, WO 95/34656, EP-A 427529, EP-A 451878, WO 03/18810 and WO 03/52073. The methods for producing such transgenic plants are generally known to the person skilled in the art and some of which are commercially available such as Agrisure®CB (P1) (corn producing Cry1Ab), Agrisure®RW (P2) (corn producing mCry3A), Agrisure® Viptera (P3) (corn hybrids producing Vip3Aa); Agrisure300GT (P4) (corn hybrids producing Cry1Ab and mCry3A); YieldGard® (P5) (corn hybrids producing the Cry1Ab protein), YieldGard® Plus (P6) (corn hybrids producing Cry1Ab and Cry3Bb1), Genuity® SmartStax® (P7) (corn hybrids with Cry1A.105, Cry2Ab2, Cry1F, Cry34/35, Cry3Bb); Herculex® I (P8) (corn hybrids producing Cry1Fa) and Herculex®RW (P9) (corn hybrids producing Cry34Ab1, Cry35Ab1 and the enzyme Phosphinothricin-N-Acetyltransferase [PAT]); NuCOTNO33B (P10) (cotton cultivars producing Cry1Ac), Bollgard®I (P11) (cotton cultivars producing Cry1Ac), Bollgard®II (P12) (cotton cultivars producing Cry1Ac and Cry2Ab2) and VIPCOT® (P13) (cotton cultivars producing a Vip3Aa). Soybean Cyst Nematode resistance soybean (SCN®—Syngenta (P14)) and soybean with Aphid resistant trait (AMT® (P15)) are also of interest.

Further examples of such transgenic crops are:

1. Bt11 Maize from Syngenta Seeds SAS, Chemin de l'Hobit 27, F-31 790 St. Sauveur, France, registration number C/FR/96/05/10 (P16). Genetically modified Zea mays which has been rendered resistant to attack by the European corn borer (Ostrinia nubilalis and Sesamia nonagrioides) by transgenic expression of a truncated CryIA(b) toxin. Btl 1 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10 (P17). Genetically modified Zea mays which has been rendered resistant to attack by the European corn borer (Ostrinia nubilalis and Sesamia nonagrioides) by transgenic expression of a CryIA(b) toxin. Btl 76 maize also transgenically expresses the enzyme PAT to achieve tolerance to the herbicide glufosinate ammonium.

3. MIR604 Maize from Syngenta Seeds SAS, Chemin de l′Hobit 27, F-31 790 St. Sauveur, France, registration number C/FR/96/05/10 (P18). Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9 (P19). MON 863 expresses a CryIIIB(b1) toxin and has resistance to certain Coleoptera insects.

5. IPC 531 Cotton from Monsanto Europe S.A. 270-272 Avenue de Tervuren, B-1150 Brussels, Belgium, registration number C/ES/96/02. (P20)

6. 1507 Maize from Pioneer Overseas Corporation, Avenue Tedesco, 7 B-1160 Brussels, Belgium, registration number C/NL/00/10. (P21) Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Further examples of transgenic plants, and of very high interest, are those carrying traits conferring resistance to 2.4D (e.g. Enlist®) (e.g. WO 2011066384) (glyphosate (e.g. Roundup Ready® (P24), Roundup Ready 2 Yield® (P25)), sulfonylurea (e.g. STS®) (P26), glufosinate (e.g. Liberty Link® (P27), Ignite® (P28)), Dicamba (P29) (Monsanto), HPPD tolerance (P30) (e.g. isoxaflutole herbicide) (Bayer CropScience, Syngenta). Double or triple stacks of any of the traits described here are also of interest, including glyphosate and sulfonyl-urea tolerance ((e.g. Optimum GAT®) (P31), plants stacked with STS® and Roundup Ready® (P32) or plants stacked with STS® and Roundup Ready 2 Yield® (P33)), dicamba and glyphosate tolerance (P34) (Monsanto). Of particular interest are soybean plants carrying trains conferring resistance to 2.4D (e.g. Enlist®), glyphosate (e.g. Roundup Ready®, Roundup Ready 2 Yield®), sulfonylurea (e.g. STS®), glufosinate (e.g. Liberty Link®, Ignite®), Dicamba (Monsanto) HPPD tolerance (e.g. isoxaflutole herbicide) (Bayer CropScience, Syngenta).

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

Examples of cotton transgenic events include MON 531/757/1076 (Bollgard I®—Monsanto), MON1445 (Roundup ready Cotton®—Monsanto), MON531×MON1445 (Bollgard I+RR®—Monsanto), MON15985 (Genuity Bollgard II Cotton®—Monsanto), MON88913 (Genuity RR FLEX Cotton®—Monsanto), MON15985×MON1445 (Genuity Bollgard II+RR FELX Cotton®—Monsanto), MON15983×MON88913 (Genuity Bollgard II+RR FLEX Cotton®—Monsanto), MON15985 (FibreMax Bollgard II Cotton®—Monsanto), LL25 (FibreMax LL Cotton®—BCS Stoneville), GHB614 (FibreMax GlyTol Cotton®—BCS Stoneville), LL25×MON15985 (FibreMax LL Bollgard II cotton® —BCS Stoneville/Monsanto), GHB614×LL25 (FibreMax LL GlyTol Cotton®—BCS Stoneville), GHB614×LL25×MON15985 (FibreMax RR GlyTol Bollgard II Cotton®—BCS Stoneville), MON88913×MON15985 (FibreMax LL GlyTol Bollgard II Cotton®—Monsanto), MON88913 (FibreMax RR Flex Cotton®—Monsanto), GHB119+T304-40 (Twinlink®—BCS Stoneville), GHB119+T304-40×LL25×GHB614 (Twinlink LL GT®—BCS Stoneville), 3006-210-23×281-24-236 (PhytoGen Widestrike Insect Protection®—Dow), 3006-210-23×281-24-236×MON88913 (PhytoGen Widestrike Insect Protection+RR FLEX —® Dow/Monsanto), 3006-210-23×281-24-236×MON1445 ((PhytoGen Widestrike Insect Protection+RR®—Dow/Monsanto), MON1445 (PhytoGen Roundup Ready®—Monsanto), MON88913 (PhytoGen Roundup Ready FLEX®—Monsanto), COT102×COT67B (Vipcot®—Syngenta), COT102×COT67B×MON88913 (Vipcot RR FLEX®—Syngenta/Monsanto), 281-24-236 (Dow), 3006-210-23 (Dow), COT102 (Syngenta), COT67B (Syngenta), T304-40 (BCS Stoneville).

Examples of Soy transgenic events include MON87701×MON89788 (Genuity Roundup ready 2 Yield Soybeans®—Monsanto), MON89788 (Roundup Ready2Yield®, RR2Y®—Monsanto), MON87708 (Monsanto), 40-3-2 (Roundup Ready®, RR1®—Monsanto), MON87701 (Monsanto), DAS-68416 (Enlist Weed Control System®—Dow), DP356043 (Optimum GAT®—Pioneer), A5547-127 (LibertyLink Soybean®—Bayercropscience), A2704-12 (Bayercropscience), GU262 (Bayercropscience), W62 W98 (Bayercropscience), CRV127 (Cultivance®—BASF/EMBRAPA), SYHT0H2 (WO2012/082548).

Examples of Maize transgenic events include T25 (LibertyLink®, LL®—Bayerscropscience), DHT-1 (Dow), TC1507 (Herculex I®—Dow), DAS59122-7 (Herculex RW®—Dow), TC1507+DAS59122-7—Herculex Xtra®—Dow), TC1507×DAS-59122-7×NK603 (Herculex Xtra+RR® —Dow), TC1507×DAS-59122-×MON88017×MON89034 (Genuity Smartstax Corn®, Genuity Smartstax RIB Complete®—Monsanto/Dow), MON89034×NK603 (Genuity VT double PRO®—Monsanto), MON89034+MON88017 (Genuity VT Triple PRO®—Monsanto), NK603 (Roundup Ready 2®, RR2®—Monsanto), MON810 (YieldGard BT®, Yieldgard Cornborer®—Monsanto), MON810×NK603 (YieldGard cornborer RR Corn 2®—Monasnto), MON810×MON863 (YieldGard Plus® —Monsanto), MON863×MON810×NK603 (YieldGard Plus+RR Corn2®/YieldGard RR Maize® —Monsanto), MON863×NK603 (YieldGard Rotworm+RR Corn 2®—Monsanto), MON863 (YieldBard RW®—Monsanto), MON89034 (YieldGard RW®—Monsanto), MON88017 (YieldGard VT RW® —Monsanto), MON810+MON88017 (YieldGard VT Triplet —Monsanto), MON88017+MON89034 (YieldGard VT Triple Pro®—Monsanto), Btl 1+MIR604+GA21 (Agrisure 3000®—Syngenta), Btl 1+TC1507+MIR604+5307+GA21 (Syngenta), Bt11+TC1507+MIR604+DAS59122+GA21 (Agrisure 3122®—Syngenta), BT11 (Agrisure CB®—Syngenta), GA21—(Agrisure GT®—Syngenta), MIR604 (Agrisure RW®—Syngenta), Bt11+MIR162 (Agrisure TL VIP®—Syngenta), BT11+MIR162+GA21 (Agrisure Viptra 31100—Syngenta), BT11+MIR162+MIR604 (Agrisure™ 3100® —Syngenta), Event3272+BT11+MIR604+GA21 (Syngenta), BT11+MIR1692+MIR604+GA21 (Agrisure Viptera 3111®—Syngenta), BT11+MIR 162+TC1507+GA21 (Agrisure Viptera 3220® —Syngenta), BT11+MIR162+TC1507+MIR604+5307+GA21 (Agrisure Viptera 3222®—Syngenta), MIR162 (Syngenta), BT11+GA21+MIR162+MIR604+5307 (Syngenta), 5307 (Syngenta).

Herbicide-resistant plants (plants bred in a conventional manner for herbicide tolerance) which may be mentioned include the varieties sold under the name Clearfield(®) (for example maize). These statements also apply to plant cultivars having these genetic traits or genetic traits still to be developed, which plant cultivars will be developed and/or marketed in the future.

The compounds of the invention are suitable for use on any cotton plant, including those that have been genetically modified to be resistant to active ingredients such as herbicides, or to produce biologically active compounds that control infestation by plant pests, e.g. BT cotton.

A compound of the invention may be used in mixtures with fertilizers (for example nitrogen-, potassium- or phosphorus-containing fertilizers). Suitable formulation types include granules of fertilizer. The mixtures preferably contain up to 25% by weight of the compound of the invention.

The invention therefore also provides a fertilizer composition comprising a fertilizer and a compound of the invention.

The compositions of this invention may contain other compounds having biological activity, for example micronutrients or compounds having fungicidal activity or which possess plant growth regulating, herbicidal, insecticidal, nematicidal or acaricidal activity.

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, e.g. a insecticide, fungicide or herbicide, or a synergist or plant growth regulator where appropriate. An additional active ingredient may provide a composition having a broader spectrum of activity or increased persistence at a locus; synergize the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin and gamma cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin, acrinathirin, etofenprox or 5-benzyl-3-furylmethyl-(E)-(1R,3S)-2,2-dimethyl-3-(2-oxothiolan-3-ylidenemethyl)cyclopropane carboxylate;
b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron, diafenthiuron, lufeneron, novaluron, noviflumuron or chlorfluazuron;
e) Organic tin compounds, such as cyhexatin, fenbutatin oxide or azocyclotin;
f) Pyrazoles, such as tebufenpyrad, tolfenpyrad, ethiprole, pyriprole, fipronil, and fenpyroximate;
g) Macrolides, such as avermectins or milbemycins, for example abamectin, emamectin benzoate, ivermectin, milbemycin, spinosad, azadirachtin, milbemectin, lepimectin or spinetoram;
h) Hormones or pheromones;
i) Organochlorine compounds, such as endosulfan (in particular alpha-endosulfan), benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds, such as imidacloprid, thiacloprid, acetamiprid, nitenpyram, dinotefuran, thiamethoxam, clothianidin, or nithiazine;
m) Diacylhydrazines, such as tebufenozide, chromafenozide or methoxyfenozide;
n) Diphenyl ethers, such as diofenolan or pyriproxifen;
o) Ureas such as Indoxacarb or metaflumizone;
p) Ketoenols, such as Spirotetramat, spirodiclofen or spiromesifen;
q) Diamides, such as flubendiamide, chlorantraniliprole (Rynaxypyr®) or cyantraniliprole;
r) Essential oils such as Bugoil®—(PlantImpact); or
s) a comopund selected from buprofezine, flonicamid, acequinocyl, bifenazate, cyenopyrafen, cyflumetofen, etoxazole, flometoquin, fluacrypyrim, fluensulfone, flufenerim, flupyradifuone, harpin, iodomethane, dodecadienol, pyridaben, pyridalyl, pyrimidifen, flupyradifurone, 4-[(6-Chloro-pyridin-3-ylmethyl)-(2,2-difluoro-ethyl)-amino]-5H-furan-2-one (DE 102006015467), CAS: 915972-17-7 (WO 2006129714; WO2011/147953; WO2011/147952), CAS: 26914-55-8 (WO 2007020986), chlorfenapyr, pymetrozine, sulfoxaflor and pyrifluqinazon.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704) (e.g. acibenzolar-S-methyl), alanycarb, aldimorph, anilazine, azaconazole, azoxystrobin, benalaxyl, benomyl, benthiavalicarb, biloxazol, bitertanol, bixafen, blasticidin S, boscalid, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cyclufenamid, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)-N-benzyl-N-([methyl(methyl-thioethylideneamino-oxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluopyram, fluoxastrobin, fluoroimide, fluquinconazole, flusilazole, flutolanil, flutriafol, fluxapyroxad, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, isopyrazam, kasugamycin, kresoxim-methyl, LY186054, LY211795, LY248908, mancozeb, mandipropamid, maneb, mefenoxam, metalaxyl, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, penflufen, penthiopyrad, phenazin oxide, phosetyl-A1, phosphorus acids, phthalide, picoxystrobin (ZA1963), polyoxinD, polyram, probenazole, prochloraz, procymidone, propamocarb, propiconazole, propineb, propionic acid, prothioconazole, pyrazophos, pyrifenox, pyrimethanil, pyraclostrobin, pyroquilon, pyroxyfur, pyrrolnitrin, quaternary ammonium compounds, quinomethionate, quinoxyfen, quintozene, sedaxane, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram, N-[9-(dichloromethylene)-1,2,3,4-tetrahydro-1,4-methanonaphthalen-5-yl]-3-(difluoromethyl)-1-methyl-1H-pyrazole-4-carboxamide[1072957-71-1], 1-methyl-3-difluoromethyl-1H-pyrazole-4-carboxylic acid (2-dichloromethylene-3-ethyl-1-methyl-indan-4-yl)-amide, and 1-methyl-3-difluoromethyl-4H-pyrazole-4-carboxylic acid [2-(2,4-dichloro-phenyl)-2-methoxy-1-methyl-ethyl]-amide.

In addition, biological agents may be included in the composition of the invention e.g. Bacillus species such as Bacillus firmus, Bacillus cereus, Bacillus subtilis, and Pasteuria species such as Pasteuria penetrans and Pasteuria nishizawae. A suitable Bacillus firmus strain is strain CNCM 1-1582 which is commercially available as BioNem™. A suitable Bacillus cereus strain is strain CNCM 1-1562. Of both Bacillus strains more details can be found in U.S. Pat. No. 6,406,690. Other biological organisms that may be included in the compositions of the invention are bacteria such as Streptomyces spp. such as S. avermitilis, and fungi such as Pochonia spp. such as P. chlamydosporia. Also of interest are Metarhizium spp. such as M. anisopliae; Pochonia spp. such as P. chlamydosporia.

The compounds of the invention may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

Unless otherwise stated the weight ratio of the compound of I with an additional active ingredient may generally be between 1000:1 and 1:1000. In other embodiments that weight ratio of A to B may be between 500:1 to 1:500, for example between 100:1 to 1:100, for example between 1:50 to 50:1, for example 1:20 to 20:1, for example 1:10 to 10:1, for example 1:5 to 5:1, for example 1:1, 1:2, 1:3, 1:4, 1:5, 2:1, 3:1, 4:1, or 5:1.

Mixtures with pyrethroids, in particular pymetrozine, are of particular interest for the present invention.

Compositions of the invention include those prepared by premixing prior to application, e.g. as a readymix or tankmix, or by simultaneous application or sequential application to the plant.

In order to apply a compounds of the invention as an insecticide, acaricide, nematicide or molluscicide to a pest, a locus of pest, or to a plant susceptible to attack by a pest, compounds of the invention is usually formulated into a composition which includes, in addition to the compound of the invention, a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of the invention. The composition is generally used for the control of pests such that a compound of the invention is applied at a rate of from 0.1 g to 10 kg per hectare, preferably from 1 g to 6 kg per hectare, more preferably from 1 g to 1 kg per hectare.

In one embodiment the compounds of the invention are used for pest control on cotton at 1:500 g/ha, for example 10-70 g/ha. However, it should be noted that due to the very damaging effect of the Anthonomus grandis (quantity and quality on yield), sprays are often very intense and at very low threshold levels and can be down to almost zero tolerance.

When used in a seed dressing, a compound of the invention is used at a rate of 0.0001 g to 10 g (for example 0.001 g or 0.05 g), preferably 0.005 g to 10 g, more preferably 0.005 g to 4 g, per kilogram of seed.

Compositions comprising a compound of the invention can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), microemulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of the invention.

Dustable powders (DP) may be prepared by mixing a compound of the invention with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of the invention with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the invention and one or more powdered solid diluents or carriers, or from pre-formed blank granules by absorbing a compound of the invention (or a solution thereof, in a suitable agent) in a porous granular material (such as pumice, attapulgite clays, fuller's earth, kieselguhr, diatomaceous earths or ground corn cobs) or by adsorbing a compound of the invention (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of the invention in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of the invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of the invention either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the invention is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the invention. SCs may be prepared by ball or bead milling the solid compound of the invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the invention may be dry milled and added to water, containing agents hereinbefore described, to produce the desired end product.

Aerosol formulations comprise a compound of the invention and a suitable propellant (for example n-butane). A compound of the invention may also be dissolved or dispersed in a suitable medium (for example water or a water miscible liquid, such as n-propanol) to provide compositions for use in non-pressurized, hand-actuated spray pumps.

A compound of the invention may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of the invention and, optionally, a carrier or diluent therefor. The polymeric shell may be produced by either an interfacial polycondensation reaction or by a coacervation procedure. The compositions may provide for controlled release of the compound of the invention and they may be used for seed treatment. A compound of the invention may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of the invention). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of the invention).

A compound of the invention may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of the invention may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of the invention may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of the invention (for example 0.0001 to 10%, by weight) depending upon the purpose for which they are to be used.

The following Examples illustrate but do not limit the invention.

### EXAMPLE 1

### Preparation of N-[(1S)-1-[4-[3-[3,5-bis(trifluoromethyl)phenyl]-3-(trifluoromethyl)pyrrolidin-1-yl]phenyl]ethyl]cyclopropanecarboxamide

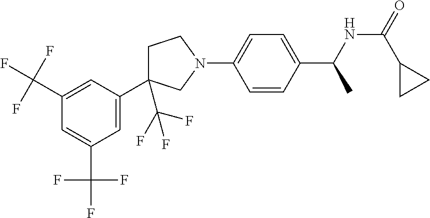 [SMILES: C[C@H](NC(=O)C1CC1)c1ccc(N2CCC(c3cc(C(F)(F)F)cc(C(F)(F)F)c3)(C(F)(F)F)C2)cc1]

To a solution of 3-[3,5-bis(trifluoromethyl)phenyl]-3-(trifluoromethyl)pyrrolidine (0.15 g, 0.4271 mmol, prepared as described in WO 2008/128711) and N-[(1S)-1-(4-bromophenyl)ethyl]cyclopropanecarboxamide (0.134 g, 0.4997 mmol, prepared as described in WO 2012/001107) in Toluene (6.03 mL), stirred under argon were added tris(dibenzylideneacetone)dipalladium(0) (9 mg), 4,5-Bis(diphenylphosphino)-9,9-dimethylxanthene (18 mg) and sodium tert-butoxide (96 mg). The mixture was heated in a microwave at 130° C. for 15 min. The reaction was then diluted with ethylacetate and water then brine and then the mixture was extracted with ethyl acetate. The organic layers were combined and dried over magnesium sulphate, filtered then concentrated under reduced pressure to give a yellow oil which was purified by chromatography on a column (cyclohexane/EtOAc as solvent) to afford the desired product as a white foam (172 mg).

¹H NMR (CDCl3, 400 MHz): d=7.92 (s, 1H), 7.86 (s, 2H), 7.27 (m, 2H), 6.63 (d, J=8.4 Hz, 2H), 5.74 (d, J=7.7 Hz, 1H), 5.09 (t, J=7.3 Hz, 1H), 4.16 (d, J=10.3 Hz, 1H), 3.85 (d, J=10.3 Hz, 1H), 3.60 (d, J=8.1 Hz, 1H), 3.51 (d, J=2.9 Hz, 1H), 2.91-3.03 (m, 1H), 2.61 (d, J=13.6 Hz, 1H), 1.50 (d, J=7.0 Hz, 3H), 1.17-1.34 (m, 1H), 0.90-1.04 (m, 2H), 0.63-0.80 ppm (m, 2H)

### EXAMPLE 2

### Preparation of N-[(1S)-1-[4-[3-(3,5-dichlorophenyl)-3-(trifluoromethyl)pyrrolidin-1-yl]phenyl]ethyl]cyclopropanecarboxamide

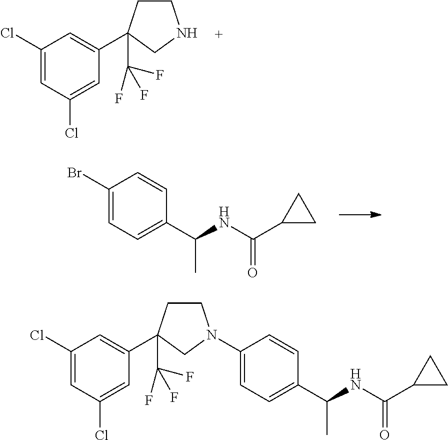

Tris(dibenzylideneacetone)dipalladium(0) (32 mg), 4,5-bis(diphenylphosphino)-9,9-dimethylxanthene (61 mg) and sodium tert-butoxide (190 mg) were added to a solution of 3-(3,5-dichlorophenyl)-3-(trifluoromethyl)pyrrolidine (520 mg, prepared as described in WO 2008/128711) and N-[(1S)-1-(4-bromophenyl)ethyl]cyclopropanecarboxamide (prepared as described in WO 2012/001107, 520 mg) in toluene (15 mL) under argon atmosphere. The mixture was heated in a microwave at 130° C. for 30 min. The reaction was then diluted with ethylacetate and water then brine and then the mixture was extracted with ethyl acetate. The organic layers were combined and dried over sodium sulphate, filtered then concentrated under reduced pressure to give a yellow oil which was purified by chromatography on column (Heptane/EtOAc as solvent (10/0 to 0/10) to afford the desired product as a white foam (650 mg).

¹H NMR (CDCl3, 400 MHz): d=7.40 (s, 1H), 7.31 (s, 2H), 7.26 (m, 2H), 6.62 (d, 2H), 5.73 (d, 1H), 5.09 (t, 1H), 4.06 (d, 1H), 3.85 (d, 1H), 3.57 (m, 1H), 3.50 (m, 1H), 2.84 (m, 1H), 2.56 (m, 1H), 1.50 (d, 3H), 1.2 (m, 1H), 0.99 (m, 2H), 0.72 ppm (m, 2H)

### EXAMPLE 3

### Preparation of 1-[4-[5-(3,5-dichlorophenyl)-5-(trifluoromethyl)-4H-isoxazol-3-yl]phenyl]ethyl]acetamide

### Step 1:

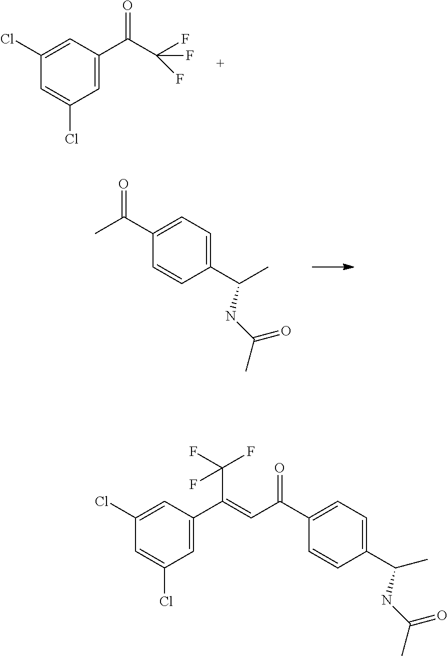 [SMILES: O=C(c1cc(Cl)cc(Cl)c1)C(F)(F)F ; CC(=O)N[C@@H](C)c1ccc(C(C)=O)cc1 ; CC(=O)N[C@@H](C)c1ccc(C(=O)/C=C(/c2cc(Cl)cc(Cl)c2)C(F)(F)F)cc1]

To a suspension of N-[(1S)-1-(4-acetylphenyl)ethyl]acetamide (3 g) and 1-(3,5-dichlorophenyl)-2,2,2-trifluoro-ethanone (3.6 g) in 1,2-dichloroethane (40 mL) was added triethylamine (0.2 mL) and potassium carbonate (1 g). The solution was stirred at 80° C. After 40 minutes more potassium carbonate (1 g) was added. The solution was heated at 80° C. for another 20 minutes then more potassium carbonate (1 g) was added and the suspension was refluxed for 16 hours. The mixture was then cooled to room temperature, and water was added. The mixture was extracted with dichloromethane then washed with brine. The organic layers were combined and dried over magnesium sulphate, filtered then concentrated under reduced pressure to give a yellow oil which was purified by chromatography on column (Heptane/EtOAc as solvent (1/0 to 3/7) to afford the desired product as a yellow oil (5 g).

1H NMR (CDCl3, 400 MHz): d=7.78-7.87 (m, 2H), 7.42-7.40 (m, 2H), 7.33 (m, 1H), 7.16 (d, 2H), 5.69 (d, 1H), 5.02-5.26 (m, 1H), 2.02 (s, 3H), 1.48-1.51 ppm (d, 3H)

### Step 2:

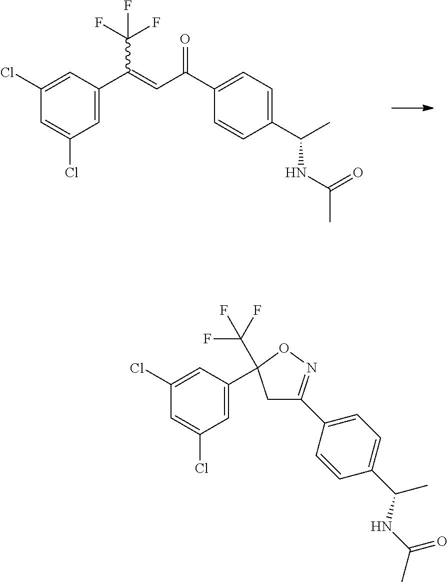 [SMILES: CC(=O)N[C@@H](C)c1ccc(C(=O)C=C(c2cc(Cl)cc(Cl)c2)C(F)(F)F)cc1 ; CC(=O)N[C@@H](C)c1ccc(C2=NOC(c3cc(Cl)cc(Cl)c3)(C(F)(F)F)C2)cc1]

To a solution of N-[(1S)-1-[4-[3-(3,5-dichlorophenyl)-4,4,4-trifluoro-but-2-enoyl]phenyl]ethyl]acetamide (100 mg) in 1,2-dichloroethane (4 mL) was added tetrabutylammonium hydrobromide (40 mg), hydroxylamine (0.03 mL, 50% in water) and sodium hydroxide (0.46 mL, 1M) at room temperature. The solution was stirred at room temperature for 6 hours then a solution of saturated ammonium chloride was added. The mixture was extracted with DCM then washed with brine. The organic layers were combined and dried over magnesium sulphate, filtered then concentrated under reduced pressure to give a yellow oil which was purified by chromatography on column (Heptane/EtOAc as solvent (1/0 to 1/1) to afford the desired product as a yellow oil (77 mg).

¹H NMR (CDCl3, 400 MHz): d=7.59-7.69 (m, 2H), 7.49-7.57 (m, 2H), 7.43 (t, J=1.8 Hz, 1H), 7.34-7.41 (m, J=8.4 Hz, 2H), 5.65 (d, J=7.3 Hz, 1H), 5.15 (t, J=7.2 Hz, 1H), 4.01-4.13 (m, 1H), 3.68 (d, J=17.2 Hz, 1H), 2.01 (s, 3H), 1.50 (d, J=7.0 Hz, 3H), 1.27 ppm (t, J=7.2 Hz, 1H).

### EXAMPLE 4

### Preparation of 2-(1,2,4-triazol-1-yl)-5-[3-(3,4,5-trichlorophenyl)-3-(trifluoromethyl)-2,4-dihydropyrrol-5-yl]benzonitrile

### Step 1: Preparation of 3-bromo-4-fluoro-N-(trimethylsilylmethyl)benzamide

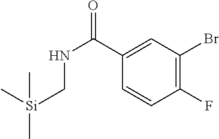 [SMILES: C[Si](C)(C)CNC(=O)c1ccc(F)c(Br)c1]

To a solution of 3-bromo-4-fluoro-benzoic acid (500 mg, 2.2830 mmol) in dichloromethane (15 mL) was added N,N-dimethylpyridin-4-amine (28 mg, 0.22830 mmol), EDCI HCl (570 mg, 2.9679 mmol). To this was added trimethylsilylmethanamine (260 mg, 2.5113 mmol) and the reaction was stirred at room temperature under nitrogen atmosphere overnight and monitored by TLC. The reaction mass was diluted with water (10 mL) and extracted with DCM (3×50 mL). The combined organic layer was dried over sodium sulphate and concentrated under vacuum. Purification by chromatography (hexane/ethyl acetate) provided 3-bromo-4-fluoro-N-(trimethylsilylmethyl)benzamide (550 mg).

¹H NMR (400 MHz, CDCl3): 7.94 (dd, 1H); 7.65 (m, 1H); 7.15 (t, 1H); 5.9 (brs, 1H); 2.94 (d, 2H); 0.12 (s, 9H), LCMS (methanol, ESI): retention time=2.06, m/z=302.0 (M−H)

### Step 2: Preparation of 3-cyano-4-fluoro-N-(trimethylsilylmethyl)benzamide

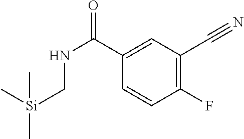 [SMILES: C[Si](C)(C)CNC(=O)c1ccc(F)c(C#N)c1]

In a sealed tube was taken a solution of 3-bromo-4-fluoro-N-(trimethylsilylmethyl)benzamide (10 g, 32.870 mmol) in N,N-dimethylformamide (60 mL) was added zinc formonitrile (7.85 g, 65.740 mmol) followed by palladium(0)tetrakis(triphenylphosphine) (7.61 g, 6.5740 mmol). The reaction was degassed and purged with nitrogen and stirred at 100° C. for 4-5 hours. The reaction was diluted with water (100 mL) extracted with ethylacetate (3×100 mL), and washed with sodium bicarbonate (50 mL). The combined organic layer was dried over sodium sulphate and concentrated under vacuum. Purification by chromatography (hexane/ethyl acetate) provided 3-cyano-4-fluoro-N-(trimethylsilylmethyl)benzamide (6.7 g)

¹H NMR (400 MHz, CDCl3): 7.98-8.02 (m, 2H); 7.3 (m, 1H); 5.94 (brs, 1H); 2.95-2.98 (m, 2H); 0.13 (s, 9H). LCMS (methanol, APCI): retention time=4.11, m/z=249.09 (M−H)

### Step 3: Preparation of 3-cyano-4-fluoro-N-(trimethylsilylmethyl)benzenecarbothioamide

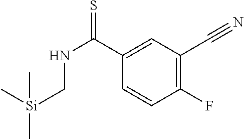 [SMILES: C[Si](C)(C)CNC(=S)c1ccc(F)c(C#N)c1]

A solution of 3-cyano-4-fluoro-N-(trimethylsilylmethyl)benzamide (6.5 g, 26 mmol) and 2,4-bis(4-methoxyphenyl)-2,4-dithioxo-1,3,2,4-dithiadiphosphetane (11 g, 26 mmol) in tetrahydrofuran (75 mL) was refluxed for 2 hours. The reaction mass was concentrated to remove THF, diluted with water (50 mL), and extracted with ethylacetate (3×100 mL). The combined organic layer was dried over sodium sulphate and concentrated under vacuum. Purification by chromatography (hexane/ethyl acetate) provided 3-cyano-4-fluoro-N-(trimethylsilylmethyl)benzenecarbothioamide (4.8 g).

¹H NMR (400 MHz, CDCl3): 8.2 (m, 1H); 7.93-7.99 (m, 1H); 7.64 (brs, 1H); 7.22 (m, 1H); 3.52 (d, 2H); 0.18 (s, 9H), LCMS (methanol, APCI): retention time=4.55, m/z=265.45 (M−H).

### Step 4: Preparation of 2-fluoro-5-[3-(3,4,5-trichlorophenyl)-3-(trifluoromethyl)-2,4-dihydropyrrol-5-yl]benzonitrile

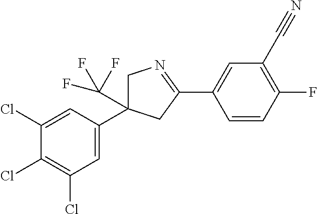 [SMILES: N#Cc1cc(C2=NCC(c3cc(Cl)c(Cl)c(Cl)c3)(C(F)(F)F)C2)ccc1F]

To a solution of 3-cyano-4-fluoro-N-(trimethylsilylmethyl)benzenecarbothioamide (4 g, 15.02 mmol) in N,N-dimethylformamide (40 mL) was added dipotassium carbonic acid (5.26 g, 37.54 mmol). To this was added iodomethane (21.31 g, 150.2 mmol). The reaction mass was stirred at room temperature for 3 hours and monitored by TLC. The reaction mass was quenched with water (10 mL) and extracted with ethylacetate (3×50 mL). The combined organic layer was dried over sodium sulphate and concentrated under vacuum to give methyl-3-cyano-4-fluoro-N-(trimethylsilylmethyl)benzenecarboximidothioate.

To a −5° C. cooled solution of methyl-3-cyano-4-fluoro-N-(trimethylsilylmethyl)benzenecarboximidothioate (4 g, 14.27 mmol) and 1,2,3-trichloro-5-[1-(trifluoromethyl)vinyl]benzene (3.97 g., 14.41 mmol) in tetrahydrofuran (5 mL) was slowly added tetrabutylammonium hydrofluoride (7.13 mL, 7.133 mmol, 1 mol/L). The reaction was stirred at −5° C. for 30 mins, then allowed to come to room temperature, and stirred at room temperature for 2 hours under nitrogen atmosphere. The reaction was diluted with water (50 mL) and extracted with ethylacetate (3×100 mL). The combined organic layer was dried over sodium sulphate and concentrated under vacuum. Purification by chromatography (hexane/ethyl acetate) provided 2-fluoro-5-[3-(3,4,5-trichlorophenyl)-3-(trifluoromethyl)-2,4-dihydropyrrol-5-yl]benzonitrile (2.5 g).

¹H NMR (400 MHz, CDCl₃): 8.04-8.13 (m, 2H); 7.37 (s, 2H); 7.35 (m, 1H); 4.82 (dd, 1H); 4.36 (d, 1H); 3.7 (dd, 1H); 3.35 (d, 1H), LCMS (methanol, APCI): retention time=5.05, m/z=434.88 (M+H)

### Step 5: Preparation of 2-(1,2,4-triazol-1-yl)-5-[3-(3,4,5-trichlorophenyl)-3-(trifluoromethyl)-2,4-dihydropyrrol-5-yl]benzonitrile

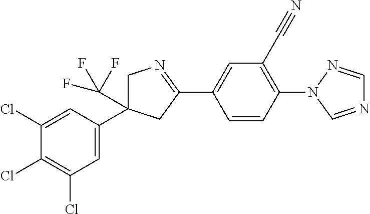 [SMILES: N#Cc1cc(C2=NCC(c3cc(Cl)c(Cl)c(Cl)c3)(C(F)(F)F)C2)ccc1-n1cncn1]

A solution of 2-fluoro-5-[3-(3,4,5-trichlorophenyl)-3-(trifluoromethyl)-2,4-dihydropyrrol-5-yl]benzonitrile (1.0 g, 2.3 mmol), dipotassium carbonic acid (390 mg, 2.8 mmol) and 1H-1,2,4-triazole (190 mg, 2.8 mmol) were heated at 120° C. for 2-3 hours and monitored by TLC. The reaction was diluted with water (10 mL) and extracted with ethylacetate (3×30 mL). The combined organic layer was dried over sodium sulphate and concentrated under vacuum. Purification by chromatography (hexane/ethyl acetate) provided 2-(1,2,4-triazol-1-yl)-5-[3-(3,4,5-trichlorophenyl)-3-(trifluoromethyl)-2,4-dihydropyrrol-5-yl]benzonitrile as solid (950 mg). Melting point: 162-164° C.

¹H NMR (400 MHz, CDCl₃): 8.9 (s, 1H), 8.29-8.34 (m, 2H); 8.21 (s, 1H); 7.93 (d, 1H); 7.40 (s, 2H); 4.94 (d, 1H); 4.51 (d, 1H); 3.84 (d, 1H); 3.54 (d, 1H); LCMS (methanol, APCI): retention time=4.96, m/z=483.94 (M+H)

### EXAMPLE 5

### Preparation of N-[(1S)-1-[4-[5-(3,4,5-trichlorophenyl)-3-(trifluoromethyl)-2,4-dihydropyrrol-3-yl]phenyl]ethyl]cyclopropanecarboxamide

### Step 1: Preparation of tert-butyl N-[(1S)-1-(4-bromophenyl)ethyl]carbamate

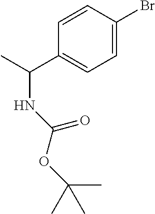 [SMILES: CC(NC(=O)OC(C)(C)C)c1ccc(Br)cc1]

To a stirring solution of compound (1S)-1-(4-bromophenyl)ethanamine (50 mmol) in a 50 mL roundbottom flask and tert-butoxycarbonyl tert-butyl carbonate (50 mmol) was added over period of 20 min and stirring was continued vigorously, After 5 min solid was precipitated out from reaction mass and thus obtained solid was filtered and washed with hexane and dried under vacuum. Weight: 13 g

¹H-NMR (400 MHz, CDCl₃): 7.45 (2H, d), 7.18 (2H, d), 4.74 (1H, m), 1.40-1.48 (12H, m).

### Step 2: Preparation of 4-[(1S)-1-(tert-butoxycarbonylamino)ethyl]benzoic acid

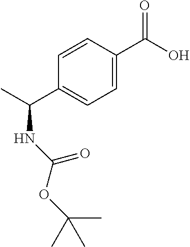 [SMILES: C[C@H](NC(=O)OC(C)(C)C)c1ccc(C(=O)O)cc1]

To a stirring solution of compound tert-butyl N-[(1S)-1-(4-bromophenyl)ethyl]carbamate (1 g, 3.331 mmol) in THF (30 mL) cooled to −78° C., methyl lithium (5.33 mmol, 1.6 eq.) was added drop wise under nitrogen atmosphere and stirring was continued for 15 mins followed by addition of butyllithium (5.33 mmol, 1.6 eq.) drop wise and stirring was continued for 30 mins Dry carbon dioxide was added and stirring continued for 1 hour at −70° C. to room temperature. The reaction mass was quenched with water (50 mL) and the compound was extracted with ethylacetate (30 mL×2) in order to remove debromo compound. The aqueous layer was acidified with ammonium chloride and extracted with ethylacetate (30 mL×2). The combined organic layers were dried with sodium sulfate and evaporated off under vacuum and dried under vacuum. Weight: 0.61 g ¹H-NMR (400 MHz, CDCl₃): 7.86 (2H, d), 7.37 (2H, d), 4.64 (1H, m), 1.28-1.31 (12H, m). LC-MS (methanol, ESI): m/z=264 (M−H), RT 1.95-2.07.

### Step 3: Preparation of tert-butyl N-[(1S)-1-[4-(trimethylsilylmethylcarbamoyl)phenyl]ethyl]carbamate

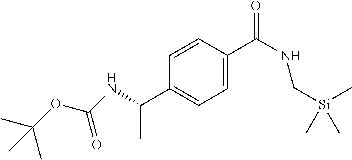 [SMILES: C[C@H](NC(=O)OC(C)(C)C)c1ccc(C(=O)NC[Si](C)(C)C)cc1]

To a stirring solution of 4-[(1S)-1-(tert-butoxycarbonylamino)ethyl]benzoic acid (1 g, 3.769 mmol), 1-hydroxybenzotriazole hydrate (542 mg, 3.5 mmol) in DMF (5 mL), in DCM (30 mL), trimethylsilylmethanamine (0.38 g, 3.7 mmol), trimethylsilylmethanamine (0.38 g, 3.7 mmol) and the reaction mass was cooled to 0° C. and 3-(ethyliminomethyleneamino)-N,N-dimethyl-propan-1-amine hydrochloride (848 mg, 4.4234 mmol) was added and the reaction was stirred at 0° C. to room temperature overnight. TLC showed the reaction was completed. The reaction mass was quenched with water (30 mL) and extracted with DCM (35 mL×2) and the combined organic layer was washed with water (20 mL×2) and dried with sodium sulfate and evaporated of under reduced pressure and compound was purified by combiflash. Weight: 0.93 g

¹H-NMR (400 MHz, CDCl₃): 7.68 (2H, d), 7.34 (2H, d), 5.93 (1H, m). 4.81 (2H, d), 2.95 (2H, d), 1.40-1.45 (12H, m). LC-MS (methanol, ESI): m/z=349 (M−H), RT 1.95-2.07.

### Step 4: Preparation of tert-butyl N-[(1S)-1-[4(trimethylsilylmethylcarbamothioyl)phenyl]ethyl]carbamate

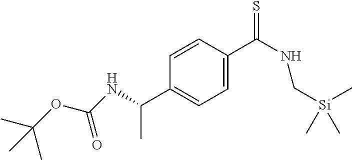 [SMILES: C[C@H](NC(=O)OC(C)(C)C)c1ccc(C(=S)NC[Si](C)(C)C)cc1]

To a solution of tert-butyl N-[(1S)-1-[4-(trimethylsilylmethylcarbamoyl)phenyl]ethyl]carbamate (1 g, 2.853 mmol) in THF (25 mL) was added 2,4-bis(4-methoxyphenyl)-1,3,2,4-dithiadiphosphetane-2,4-disulfide (1 eq, 2.853 mmol,) and reaction mass was stirred at 65° C. for 4 hours. TLC showed the reaction was completed. The solvent was removed from the reaction mass on a rotary evaporator and the resultant reaction mass was diluted with ethylacetate and washed with water (twice). The organic layer was dried over anhydrous sodium sulfate and concentrated under reduced pressure. The crude mass thus obtained was purified by combiflash (silica gel). Weight: 0.8 g

¹H-NMR (400 MHz, CDCl₃): 7.65 (2H, d), 7.51-7.52 (1H, m), 7.30 (2H, d), 4.76-4.81 (2H, m), 3.51 (2H, d), 1.40-1.45 (12H, m). LC-MS (methanol, ESI): m/z=365 (M−H), RT 2.51-2.57.

### Step 5: Preparation of tert-butyl N-[(1S)-1-[4-[5-(3,4,5-trichlorophenyl)-3-(trifluoromethyl)-2,4-dihydropyrrol-3-yl]phenyl]ethyl]carbamate

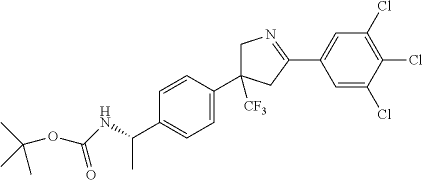 [SMILES: C[C@H](NC(=O)OC(C)(C)C)c1ccc(C2(C(F)(F)F)CN=C(c3cc(Cl)c(Cl)c(Cl)c3)C2)cc1]

To stirring solution of compound tert-butyl N-[(1S)-1-[4-(trimethylsilylcarbamothioyl)phenyl]ethyl]carbamate (0.5 g, 1 mmol) in DMF (25 mL), potassium carbonate (2 eq., 2 mmol) was added followed by addition of iodomethane (10 eq., 10 mmol) over period of 1 hour and stirring was continued for 3 ours. After completion of the reaction (monitored by TLC), the reaction mass was diluted with ethylacetate and washed with water (20 mL×3) and dried with sodium sulphate and evaporated off under vacuum. The obtained reaction mass was taken into THF (25 mL) and 1,2,3-trichloro-5-[1-(trifluoromethyl)vinyl]benzene (1 eq., 1 mmol) was added into it. The mixture was cooled to 0° C., TBAF (1 eq, 1 mmol) was added and stirring was continued for 3 hours at room temperature. After completion of the reaction (monitored by TLC), the solvent was evaporated off under vacuum and the reaction mass was diluted with ethylacetate and washed with water (30 mL×2) and dried with sodium sulphate and evaporated off under vacuum. The compound was purified by combiflash, (25-30% EtOAC-hexane). Weight: 0.11 g

¹H-NMR (400 MHz, CDCl₃): 7.97 (2H, d), 7.33-7.56 (4H, m), 4.85-4.92 (2H, m), 4.50 (1H, dd), 3.91 (1H, dd), 3.58 (1H, dd), 1.40-1.45 (12H, m). LC-MS (methanol, ESI): m/z=533 (M−H), RT 2.51-2.57.

### Step 6: Preparation of N-[(1S)-1-[4-[5-(3,4,5-trichlorophenyl)-3-(trifluoromethyl)-2,4-dihydropyrrol-3-yl]phenyl]ethyl]cyclopropanecarboxamide

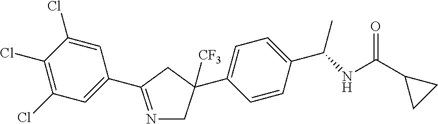 [SMILES: C[C@H](NC(=O)C1CC1)c1ccc(C2(C(F)(F)F)CN=C(c3cc(Cl)c(Cl)c(Cl)c3)C2)cc1]

To a solution of tert-butyl N-[(1S)-1-[4-[5-(3,4,5-trichlorophenyl)-3-(trifluoromethyl)-2,4-dihydropyrrol-3-yl]phenyl]ethyl]carbamate (0.35 g) in DCM (20 mL) was added 2,2,2-trifluoroacetic acid (3 eq.) and the reaction mass was stirred for 5 hours at room temperature. After completion of the reaction, the solvent was removed under vacuum and the compound thus obtained was directly taken for the next step. A Solution of (1S)-1-[4-[5-(3,4,5-trichlorophenyl)-3-(trifluoromethyl)-2,4-dihydropyrrol-3-yl]phenyl]ethanamine TFA salt (0.3 g, 0.7 mmol), in DCM (20 mL) was cooled at 0° C. To the cooled mixture was added triethylamine (2.2 eq, 1.54 mmol) followed by dropwise addition of cyclopropane carboxylic acid chloride (1 eq., 0.7 mmol) over period of 5 min and stirring was continued overnight. After completion of the reaction (monitored by TLC), the reaction mass was diluted with DCM (20 mL) and washed with water (20 mL). The organic layer was then dried over anhydrous sodium sulphate, and the solvent was removed under vacuum. The compound was purified by Combiflash (35% EtOAC-hexane). Weight: 0.2 g, melting point: 80-82° C.

¹H-NMR (400 MHz, CDCl₃): 7.82 (2H, d), 7.35-7.41 (4H, m), 6.05 (1H, m), 5.13 (1H, m), 4.86 (1H, dd), 4.42 (1H, dd), 3.78 (1H, dd), 3.45 (1H, dd). 1.46-1.50 (4H, m), 0.93-0.96 (2H, m), 0.72-0.731 (2H, m)

LC-MS (methanol, ESI): m/z=501 (M−H), RT 2.23-2.30.

### EXAMPLE 6

### Preparation of N-{2-bromo-4-[4-(3,5-dichlorophenyl)-4-trifluoromethyl-4,5-dihydro-3H-pyrrol-2yl]-benzyl}acetamide

### Step 1: Bromination of 3-bromo-4-methyl benzoic acid

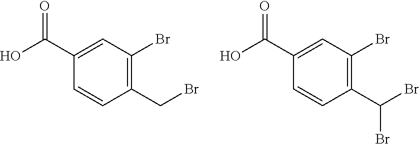 [SMILES: O=C(O)c1ccc(CBr)c(Br)c1 ; O=C(O)c1ccc(C(Br)Br)c(Br)c1]

3-bromo-4-methyl benzoic acid (10 g, 46.72 mmol), NBS (8.7 g, 49.15 mmol)) and benzoylperoxide (0.5 g, 2 mmol)) were suspended in CCl₄ and then heated to reflux for 5 hours. After completion of the reaction (TLC monitoring) water was added to the reaction mixture. The organic layer was separated, dried over anhydrous sodium sulphate and concentrated in vaccuo. The crude mixture thus obtained containing 3-bromo-4-bromomethyl benzoic acid and the 3-bromo-4-dibromomethyl benzoic acid was taken directly into the next step.

LC-MS (methanol, ESI): m/z=291 (M−H) and 368 (M−H)

### Step 2: Preparation of 3-bromo-4-(dibromomethyl)-N-[(trimethylsilyl)methyl]benzamide

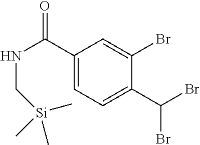 [SMILES: C[Si](C)(C)CNC(=O)c1ccc(C(Br)Br)c(Br)c1]

A mixture of of 3-bromo-4-(bromomethyl)-N-[(trimethylsilyl)methyl]benzamide and 3-bromo-4-(dibromomethyl)-N-[(trimethylsilyl)methyl]benzamide (19 g, 51 mmol), 1-(trimethylsilyl)methylamine (5.78 g, 56 mmol), N,N-dimethylaminopyridine (0.1 g) and N-(3-dimethylaminopropyl)-N′-ethylcarbodiimide.HCl (12.74 g, 66 mmol) was dissolved in tetrahydrofuran solvent and stirred for 1 hour at room temperature. After completion of the reaction (TLC monitoring), the reaction mixture was concentrated on a rotary evaporator. Water was added to the reaction mixture, followed by extraction with ethylacetate. The organic layer was separated and dried using sodium sulphate. The residue was purified by silica gel chromatography to obtain 3-bromo-4-(dibromomethyl)-N-[(trimethylsilyl)methyl]benzamide (16 g).

LC-MS (methanol, ESI): m/z=456 (M+H).

1H-NMR (400 MHz, CDCl3): 8.01 (1H, dd), 7.87 (1H, d), 7.04 (1H, dd), 6.13 (1H, bs), 3.51 (2H, d), 0.17 (9H, s).

### Step 3: Preparation of 3-bromo-4-formyl-N-[(trimethylsilyl)methyl]benzamide

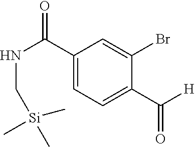 [SMILES: C[Si](C)(C)CNC(=O)c1ccc(C=O)c(Br)c1]

3-bromo-4-(dibromomethyl)-N-[(trimethylsilyl)methyl]benzamide (0.25 g, 0.69 mmol) was taken in acetone (5 mL) and water (2.5 mL). Silver nitrate (0.3 g, 1.7 mmol) was added to the reaction mixture. The reaction was stirred at room temperature for 6 hours. The reaction mass was then concentrated and extracted with ethylacetate. The organic layer was separated and dried using sodium sulphate. The organic layer was concentrated to obtain the product 3-bromo-4-formyl-N-[(trimethylsilyl)methyl]benzamide. (0.15 g).

LC-MS (methanol, ESI): m/z=314 (M+H).

1H-NMR (400 MHz, CDCl3): 10.33 (1H, s), 7.90 (1H, m), 7.60 (1H, dd), 7.56 (1H, m), 5.99 (1H, s), 2.99 (2H, d), 0.17 (9H, s).

### Step 4: Preparation of 3-bromo-4-hydroxymethyl-N-[trimethylsilaylmethyl]benzamide

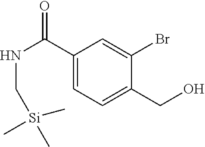 [SMILES: C[Si](C)(C)CNC(=O)c1ccc(CO)c(Br)c1]

3-bromo-4-formyl-N-[(trimethylsilyl)methyl]benzamide (0.1 g, 0.3 mmol) was dissolved in methanol and to this was added NaBH₄ (0.014 g, 0.38 mmol). The reaction was stirred at room temperature for 1 hour. Methanol was removed under vacuo and water was added to the reaction mass which was then extracted using ethyl acetate. The organic layer was separated and dried using sodium sulphate followed by concentration to get the product 3-bromo-4-hydroxymethyl-N-[trimethylsilaylmethyl]benzamide (0.05 g).

LC-MS (methanol, ESI): m/z=316 (M+H).

1H-NMR (400 MHz, CDCl3): 7.90 (1H, m), 7.60 (1H, m), 7.53 (1H, m), 6.02 (1H, s), 4.75 (2H, s), 2.99 (2H, s), 0.17 (9H, s).

### Step 5: Preparation of Acetic acid 2-bromo-4-(trimethylsilanylmethyl-carbamoyl)-benzylester

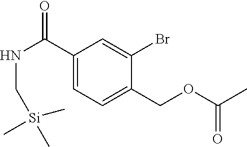 [SMILES: CC(=O)OCc1ccc(C(=O)NC[Si](C)(C)C)cc1Br]

3-bromo-4-hydroxymethyl-N-[trimethylsilaylmethyl]benzamide (0.25 g, 0.7 mmol) was dissolved in DMF solvent, triethyl amine (0.159 g, 1.5 mmol) and acetyl chloride (0.061 g, 0.8 mmol) were added to the reaction mixture at room temperature. The reaction was stirred at room temperature for 30 min. Water was added and the reaction mass was then extracted with ethyl acetate. The organic layer was separated, dried using sodium sulphate and concentrated to get the product: acetic acid 2-bromo-4-(trimethylsilanylmethyl-carbamoyl)-benzylester (0.15 g).

LC-MS (methanol): m/z=358 (M+H).

¹H-NMR (400 MHz, CDCl3): 7.90 (1H, m), 7.60 (1H, m), 7.38 (1H, m), 6.14 (1H, bs), 5.11 (2H, s), 2.99 (2H, d), 2.15 (3H, s), 0.17 (9H, s).

### Step 6: Preparation of 3-bromo-4-(2-oxo-propyl)-N-trimethylsilanylmethyl-thiobenzamide

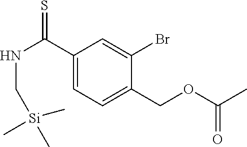 [SMILES: CC(=O)OCc1ccc(C(=S)NC[Si](C)(C)C)cc1Br]

Acetic acid 2-bromo-4-(trimethylsilanylmethyl-carbamoyl)-benzylester (2.4 g, 7.6 mmol) and Lawesson reagent (2.7 g, 6.6 mmol) were suspended in THF. The mixture was heated to reflux for 3 hours, concentrated, washed with water and extracted with ethyl acetate. The organic layer was separated and dried using sodium sulphate. The residue was purified by silica gel chromatography to obtain 3-bromo-4-(2-oxo-propyl)-N-trimethylsilanylmethyl-thiobenzamide (1.8 g).

LC-MS (methanol, ESI): m/z=374 (M+H).

¹H-NMR (400 MHz, CDCl3): 7.90 (1H, m), 7.60 (1H, m), 7.38 (1H, m), 6.01 (1H, br s), 5.11 (2H, d), 2.99 (2H, d), 2.15 (3H, s), 0.17 (9H, s)

### Step 7: Preparation of Acetic acid 2-bromo-4-[4-(3,5-dichlorophenyl)-4-trifluoromethyl-4,5-dihydro-3H-pyrrol-2yl]-benzylester

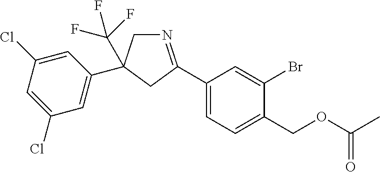 [SMILES: CC(=O)OCc1ccc(C2=NCC(c3cc(Cl)cc(Cl)c3)(C(F)(F)F)C2)cc1Br]

A mixed solution of methyliodide (0.69 g, 4.8 mmol), potassium carbonate (0.80 g, 5.7 mmol) and 3-bromo-4-(2-oxo-propyl)-N-trimethylsilanylmethyl-thiobenzamide (1.8 g, 5.4 mmol) in DMF (25 mL) was stirred at 0° C. for 4 hours. The reaction mixture was poured into ice-cold water and extracted with ethyl acetate. The organic layer was separated, dried using sodium sulphate and concentrated to get the crude acetic acid 2-bromo-4{methylsulfanyl-[(E)-trimethylsilanylmethylimino]-methyl}-benzyl ester. (0.95 g) which was dissolved in THF and cooled to 0° C. under nitrogen atmosphere. A solution of TBAF (1.0 M in THF) (0.56 mL) was added gradually into it and reaction mixture was stirred for 8 hours at room temperature. The reaction mass was then concentrated and extracted with ethylacetate. The organic layer was separated and dried using sodium sulphate. The resulting mixture was then purified by silica gel chromatography to obtain acetic acid 2-bromo-4-[4-(3,5-dichlorophenyl)-4-trifluoromethyl-4,5-dihydro-3H-pyrrol-2yl]-benzylester (1.1 g).

LC-MS (methanol, ESI): m/z=508 (M+H).

¹H-NMR (400 MHz, CDCl3): 8.07 (1H, m), 7.71 (1H, m), 7.47 (1H, m), 7.38 (1H, m), 7.24 (2H, m), 5.23 (2H, s), 4.90 (1H, m), 4.43 (1H, d), 3.75 (1H, m), 3.43 (1H, d), 2.17 (3H, s).

### Step 8: Preparation of 2-bromo-4-[4-(3,5-dichlorophenyl)-4-trifluoromethyl-4,5-dihydro-3H-pyrrol-2yl]-phenyl-methanol

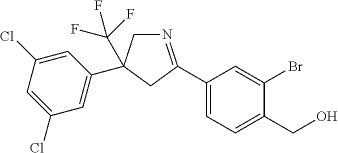 [SMILES: OCc1ccc(C2=NCC(c3cc(Cl)cc(Cl)c3)(C(F)(F)F)C2)cc1Br]

To a solution of acetic acid 2-bromo-4-[4-(3,5-dichlorophenyl)-4-trifluoromethyl-4,5-dihydro-3H-pyrrol-2yl]-benzylester (1.1 g, 2.1 mmol) in methanol (20 mL) was added sodium methoxide (0.1 g, 1.85 mmol) and the solution was stirred for 1 hour at room temperature. The reaction mass was then concentrated and extracted with ethylacetate. The organic layer was separated and dried using sodium sulphate. The resulting mixture was then purified by silica gel chromatography to obtain 2-bromo-4-[4-(3,5-dichlorophenyl)-4-trifluoromethyl-4,5-dihydro-3H-pyrrol-2yl]-phenyl-methanol (0.95 g).

LC-MS (methanol, ESI): m/z=466 (M+H).

¹H-NMR (400 MHz, CDCl3): 8.07 (1H, m), 7.78 (1H, m), 7.59 (1H, m), 7.38 (3H, m), 4.90 (3H, d), 4.43 (1H, d), 3.75 (1H, m), 3.43 (1H, d).

### Step 9: Preparation of Methanesulfonic acid-2-bromo-4-[4-(3,5-dichlorophenyl)-4-trifluoromethyl-4,5-dihydro-3H-pyrrol-2yl]-benzylester

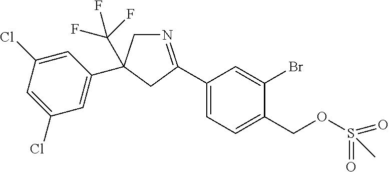 [SMILES: CS(=O)(=O)OCc1ccc(C2=NCC(c3cc(Cl)cc(Cl)c3)(C(F)(F)F)C2)cc1Br]

To a solution of 2-bromo-4-[4-(3,5-dichlorophenyl)-4-trifluoromethyl-4,5-dihydro-3H-pyrrol-2yl]-phenyl-methanol (0.95 g, 2 mmol) and triethylamine (0.4 g, 4.0 mmol) in THF was added methanesulfonyl chloride (0.35 g, 3.0 mmol) gradually. The mixture was stirred for 1 hour at room temperature. The reaction mixture was washed with water. The organic layer was separated and dried using sodium sulphate. The organic layer was evaporated to obtain the solid compound methanesulfonic acid-2-bromo-4-[4-(3,5-dichlorophenyl)-4-trifluoromethyl-4,5-dihydro-3H-pyrrol-2yl]-benzylester (0.9 g).

LC-MS (methanol, ESI): m/z=544 (M+H).

¹H-NMR (400 MHz, CDCl3): 8.13 (1H, m), 7.81 (1H, m), 7.58 (1H, m), 7.38 (1H, m), 7.24 (2H, m), 5.36 (2H, s), 4.90 (1H, m), 4.43 (1H, d), 3.75 (1H, m), 3.43 (1H, d), 3.04 (3H,$).

### Step 10: Preparation of 2-bromo-4-[4-(3,5-dichlorophenyl)-4-trifluoromethyl-4,5-dihydro-3H-pyrrol-2yl]-benzylamine

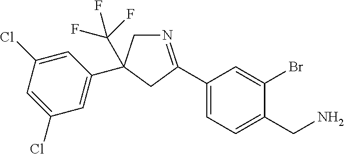 [SMILES: NCc1ccc(C2=NCC(c3cc(Cl)cc(Cl)c3)(C(F)(F)F)C2)cc1Br]

To a solution of methanesulfonic acid-2-bromo-4-[4-(3,5-dichlorophenyl)-4-trifluoromethyl-4,5-dihydro-3H-pyrrol-2yl]-benzylester (1.0 g, 1.8 mmol) in THF (30 mL) and MeOH (30 mL) was added dropwise to a mixed solution of aq. ammonia (30%), 30 mL, and the reaction was stirred for 12 hours at room temperature. The reaction mass was then concentrated and extracted with ethylacetate. The organic layer was separated and dried using sodium sulphate. The organic layer was evaporated to provide the gummy compound 2-bromo-4-[4-(3,5-dichlorophenyl)-4-trifluoromethyl-4,5-dihydro-3H-pyrrol-2yl]-benzylamine.

LC-MS (methanol, ESI): m/z=467 (M+H).

¹H-NMR (400 MHz, CDCl3): 8.07 (1H, m), 7.71 (1H, m), 7.59 (1H, m), 7.38 (1H, m), 7.24 (2H, m), 4.90 (1H, d), 4.43 (3H, m), 3.75 (1H, m), 3.43 (1H, d).

### Step 11: Preparation of N-{2-bromo-4-[4-(3,5-dichlorophenyl)-4-trifluoromethyl-4,5-dihydro-3H-pyrrol-2yl]-benzyl}acetamide

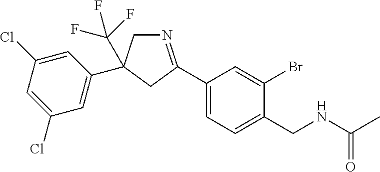 [SMILES: CC(=O)NCc1ccc(C2=NCC(c3cc(Cl)cc(Cl)c3)(C(F)(F)F)C2)cc1Br]

To a solution of 2-bromo-4-[4-(3,5-dichlorophenyl)-4-trifluoromethyl-4,5-dihydro-3H-pyrrol-2yl]-benzylamine (0.15 g, 0.32 mmol) in THF was added acetic anhydride (0.04 g, 0.39 mmol) and the mixture was stirred at room temperature for 1 hour. The reaction mass was then concentrated under reduced pressure and extracted with ethylacetate. The organic layer was separated and dried using sodium sulphate. The resulting mixture was then purified by silica gel chromatography to obtain N-{2-bromo-4-[4-(3,5-dichlorophenyl)-4-trifluoromethyl-4,5-dihydro-3H-pyrrol-2yl]-benzyl}acetamide.

LC-MS (methanol, ESI): m/z=509 (M+H).

¹H-NMR (400 MHz, CDCl3): 8.07 (1H, m), 7.71 (1H, m), 7.47 (1H, m), 7.38 (1H, m), 7.24 (2H, m), 5.98 (1H, s), 4.90 (1H, d), 4.43 (3H, m), 3.75 (1H, m), 3.43 (1H, d), 2.04 (3H, s).

### EXAMPLE 7

### Preparation of N-{4-[3-(3,5-dichlorophenyl)-3-(trifluoromethyl)pyrrolidin1-yl]-2-methyl-benzyl)acetamide

### Step 1: Preparation of 1-Benzyl-3-(3,5-dichlorophenyl)-3-(trifluoromethyl)pyrrolidine

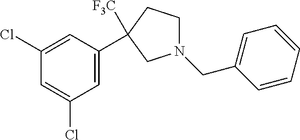 [SMILES: FC(F)(F)C1(c2cc(Cl)cc(Cl)c2)CCN(Cc2ccccc2)C1]

To a cooled solution of 1,3-dichloro-5(1-trifluoromethyl)vinyl)benzene (0.5 g, 2.0 mmol) and N-benzyl-1-methoxy-N-9trimethylsilyl)methyl)methanamine (0.4 g, 2.0 mmol) in DCM (10 mL) was added dropwise a solution of TFA (0.024 g, 0.2 mmol) in DCM (1 mL). The reaction mixture was stirred for 3 hours at room temperature. The organic layer was washed with water (2×10 mL) and 10% aq sodium carbonate solution (10 mL). The organic layer was separated, dried with sodium sulphate and concentrated under reduced pressure The residue was purified by silica gel chromatography to yield 1-Benzyl-3-(3,5-dichlorophenyl)-3-(trifluoromethyl)pyrrolidine (0.5 g).

LC-MS (methanol, ESI): m/z=374 (M+H).

¹H-NMR (400 MHz, CDCl3): 7.36 (4H, m), 7.30 (2H, m), 7.23 (2H, m), 3.67 (2H, s), 3.08 (2H, dd), 2.69 (2H, m), 2.53 (1H, m), 2.27 (1H, m).

### Step 2: Preparation of 3-(3,5-dichlorophenyl)-3-(trifluoromethyl)pyrrolidine

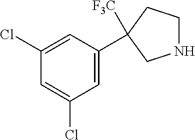 [SMILES: FC(F)(F)C1(c2cc(Cl)cc(Cl)c2)CCNC1]

To a solution of 1-benzyl-3-(-(3,5-dichlorophenyl)-3-(trifluoromethyl)pyrrolidine (0.22 g, 0.59 mmol) and 1-chloroethylchloroformate (0.17 g, 1.2 mmol) in DCM was heated for reflux for 3 hours. The mixture was cooled to room temperature and concentrated under reduced pressure. Methanol was added to the residue which was then heated with stirring for 3 hours at 60° C. The mixture was concentrated and water was added to it. The residue was extracted with ethylacetate (20 mL×3) washed with brine, dried over sodium sulphate and concentrated under reduced pressure. Purification over silica gel yielded 3-(3,5-dichlorophenyl)-3-(trifluoromethyl)pyrrolidine (4.2 g).

LC-MS (methanol, ESI): m/z=284 (M+H).

¹H-NMR (400 MHz, CDCl3): 7.35 (1H, t), 7.25 (2H, d), 3.74 (1H, d), 3.19 (2H, m), 2.97 (1H, m), 2.53 (1H, m), 2.27 (1H, m).

### Step 3: Preparation of N-(4-bromo-2-methyl-benzyl)acetamide

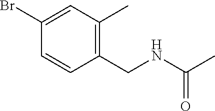

4-bromo-2-methyl benzonitrile (2.0 g, 0.01 mol) was taken in methanol along with NiCl₂ (2.41 g, 0.01 mol). In the same pot acetic anhydride was taken (2.0 g, 0.020 mol). The reaction was cooled to 0° C. NaBH₄ (2.7 g, 0.07 mol) was added slowly to the reaction while stirring at the same temperature for 1 hour. The reaction mixture was concentrated under reduced pressure and was extracted with ethylacetate. The organic layer was separated, dried using sodium sulphate and concentrated under reduced pressure. The residue thus obtained was purified by silica gel chromatography to yield N-(4-bromo-2-methyl-benzyl)acetamide. (1.1 g).

LC-MS (methanol, ESI): m/z=242 (M+H).

1H-NMR (400 MHz, CDCl3): 7.71 (1H, m), 7.47 (1H, m), 7.38 (1H, m), 5.98 (1H, s), 3.75 (1H, m), 3.43 (1H, d), 2.31 (3H, s), 2.04 (3H, s).

### Step 4: Preparation of N-{4-[3-(3,5-dichlorophenyl)-3-(trifluoromethyl)pyrrolidin1-yl]-2-methyl-benzyl)acetamide

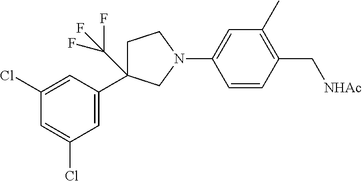 [SMILES: CC(=O)NCc1ccc(N2CCC(c3cc(Cl)cc(Cl)c3)(C(F)(F)F)C2)cc1C]

To the mixture of 3-(3,5-dichlorophenyl)-3-(trifluoromethyl)pyrrolidine (0.25 g, 0.88 mmol) and the N-(4-bromo-2-methyl-benzyl)acetamide (0.8 g, 0.7 mmol) in anhydrous toluene under nitrogen atmosphere was added tris(dibenzylidineacetone)dipalladium (0.02 g, 0.02 mmol) and the ligand xanthphos (0.03 g, 0.053 mmol) followed by the addition of sodium tertiary butoxide (0.127 g, 1.3 mmol). The reaction mixture was heated at 80° C. for 3 hours. After completion of the reaction (TLC monitoring), the reaction mass was filtered over celite. The filtrate was concentrated and purified using column chromatography.

LC-MS (methanol, ESI): m/z=445 (M+H). 1H-NMR (400 MHz, CDCl3): 7.37 (1H, d), 7.29 (2H, m), 7.11 (1H, d), 6.41 (2H, m), 5.45 (1H, b s), 4.35 (2H, s), 4.01 (1H, d), 3.75 (1H, d), 3.43 (2H, m), 2.81 (1H, d), 2.51 (1H, d), 2.31 (3H, s), 1.97 (3H, s).

The following examples were prepared according to methods described in US 2009/0156643 and were analysed by the LCMS methods described below:

### Method A:

ZQ Mass Spectrometer from Waters (Single quadrupole mass spectrometer)

### Instrument Parameter:

Ionization method: Electrospray
Polarity: positive and negative ions

### Capillary: 3.00 kV

### Cone: 30.00 V

### Extractor: 2.00 V

### Source Temperature: 100° C.,

### Desolvation Temperature: 250° C.

### Cone Gas Flow: 50 L/Hr

### Desolvation Gas Flow: 400 L/Hr

Mass range: 100 to 900 Da
HP 1100 HPLC from Agilent:
Solvent degasser, quaternary pump, heated column compartment and diode-array detector.

Type of column: Phenomenex Gemini C18; Column length: 30 mm; Internal diameter of column: 3 mm; Particle Size: 3 micron; Temperature: 60° C.

DAD Wavelength range (nm): 210 to 500

### Solvent Gradient:

### A=H2O+5% MeOH+0.05% HCOOH

### B=Acetonitril+0.05% HCOOH

| Time | A % | B % | Flow (mL/min) |
|---|---|---|---|
| 0.00 | 100 | 0 | 1.700 |
| 2.00 | 0 | 100.0 | 1.700 |
| 2.80 | 0 | 100.0 | 1.700 |
| 2.90 | 100 | 0 | 1.700 |
| 3.00 | 100 | 0 | 1.700 |

### Method B:

ACQUITY SQD Mass Spectrometer from Waters (Single quadrupole mass spectrometer) Ionisation method: Electrospray
Polarity: positive ions

### Capillary (kV) 3.00, Cone (V) 20.00, Extractor (V) 3.00, Source Temperature (° C.) 150, Desolvation

Temperature (° C.) 400, Cone Gas Flow (L/Hr) 60, Desolvation Gas Flow (L/Hr) 700

Mass range: 100 to 800 Da
DAD Wavelength range (nm): 210 to 400
Method Waters ACQUITY UPLC with the following HPLC gradient conditions
(Solvent A: Water/Methanol 9:1, 0.1% formic acid and Solvent B: Acetonitrile, 0.1% formic acid)

| Time (minutes) | A (%) | B (%) | Flow rate (mL/min) |
|---|---|---|---|
| 0 | 100 | 0 | 0.75 |
| 2.5 | 0 | 100 | 0.75 |
| 2.8 | 0 | 100 | 0.75 |
| 3.0 | 100 | 0 | 0.75 |

Type of column: Waters ACQUITY UPLC BEH C18; Column length: 50 mm; Internal diameter of column: 2.1 mm; Particle Size: 1.7 micron; Temperature: 60° C.

### EXAMPLE 8

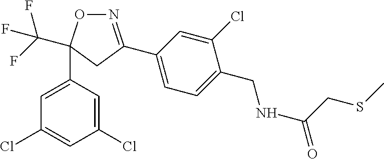 [SMILES: CSCC(=O)NCc1ccc(C2=NOC(c3cc(Cl)cc(Cl)c3)(C(F)(F)F)C2)cc1Cl]

Method A, retention time 2.17 min, MH+ 511

### EXAMPLE 9

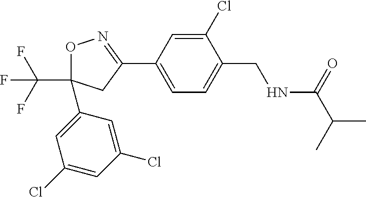 [SMILES: CC(C)C(=O)NCc1ccc(C2=NOC(c3cc(Cl)cc(Cl)c3)(C(F)(F)F)C2)cc1Cl]

Method A, retention time 2.19 min, MH+ 493

### EXAMPLE 10

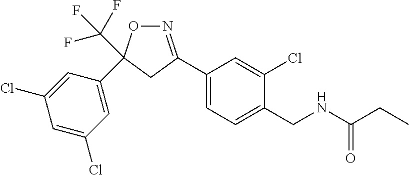 [SMILES: CCC(=O)NCc1ccc(C2=NOC(c3cc(Cl)cc(Cl)c3)(C(F)(F)F)C2)cc1Cl]

Method B, retention time 1.98 min, MH+ 479
The following Example was prepared according to methods described in WO 2008/150393.

### EXAMPLE 11

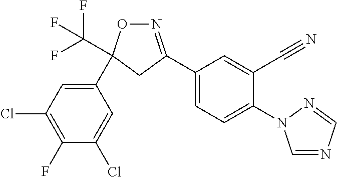 [SMILES: N#Cc1cc(C2=NOC(c3cc(Cl)c(F)c(Cl)c3)(C(F)(F)F)C2)ccc1-n1cncn1]

Method A, retention time 2.1 min, MH+ 470

The following Example was prepared according to methods described in WO 2009/051956.

### EXAMPLE 12

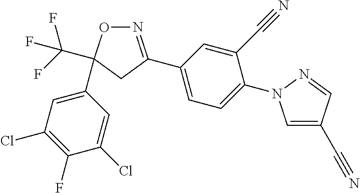 [SMILES: N#Cc1cnn(-c2ccc(C3=NOC(c4cc(Cl)c(F)c(Cl)c4)(C(F)(F)F)C3)cc2C#N)c1]

1H NMR in CDCl3: 3.73 ppm (d, 1H), 4.12 ppm (d, 1H), 7.58 ppm (d, 2H), 7.93 (m, 1H), 8.07-8.12 (m, 3H), 8.62 (s, 1H)

### EXAMPLE 13

### Preparation of 5-bromo-N-hydroxyindan-1-imine

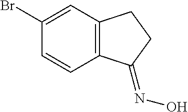 [SMILES: O/N=C1\CCc2cc(Br)ccc21]

A solution of 5-bromoindane-1-one (10.0 g, 47.38 mmol) in methanol (50 mL), treated with hydroxylamine hydrochloride (3.65 g, 52.17 mmol) and sodium acetate (4.27 g, 52.17 mmol) and stirred at room temperature for 20 hours. The solvent was evaporated, and the residue was treated with water (25 mL) and extracted with ethylacetate (2×50 mL). The combined organic layers were dried over sodium sulfate and concentrated to give 5-bromo-N-hydroxyindan-1-imine (10 g). ¹H-NMR (400 MHz, CDCl₃): 7.65 (1H d), 7.5 (1H, s), 7.4 (1H, d), 3.05 (2H, m), 3.10 (2H, m). LC-MS (methanol, ESI): m/z=227 (M+H, retention time=1.75).

### EXAMPLE 14

### Preparation of tert-butyl (5-bromo-2,3-dihydro-1H-inde-1-yl)carbamate

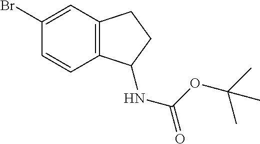 [SMILES: CC(C)(C)OC(=O)NC1CCc2cc(Br)ccc21]

A solution of 5-bromo-N-hydroxyindan-1-imine (16 g, 70.77 mmol) in methanol (400 mL) and dioxane (200 mL) was treated with di-ter-butyl bicarbonate (31 g, 141.55 mmol) and nickel chloride (4.68 g, 35.39 mmol). The reaction mixture was cooled to −20° C., sodium borohydride (10.7 g, 283.1 mmol) was added slowly and stirred for 1 hour. The reaction mixture was then treated with diethylenetriamine (16 mL), and stirred for 30 min. The reaction mixture was diluted by adding water, extracted with ethyl acetate. The combined organic layers were dried over sodium sulfate, concentrated and purified by column chromatography (hexane/ethylacetate 1:9 as eluent) to give ter-butyl (5-bromo-2,3-dihydro-1H-inde-1-yl)carbamate (10.5 g). ¹H-NMR (400 MHz, CDCl₃): 7.1 (1H, d), 7.2 (1H, s), 7.3 (1H, d), 5.15 (1H, m), 4.7 (1H, m), 2.9 (1H, m), 2.8 (1H, m), 2.5 (1H, m), 1.8 (1H, m), 1.45 (s, 9H).

### EXAMPLE 15

### Preparation of 1-(tert-butoxycarbonylamino)indane-5-carboxylic acid

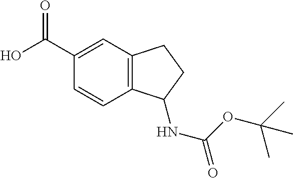 [SMILES: CC(C)(C)OC(=O)NC1CCc2cc(C(=O)O)ccc21]

A solution of ter-butyl (5-bromo-2,3-dihydro-1H-inde-1-yl)carbamate (10 g, 32 mmol) in tetrahydrofuran (200 mL) was cooled to −78° C., treated dropwise with methyl lithium (16 mL, 48 mmol) and stirred for 10 min, followed by addition of n-BuLi (29 mL, 64 mmol) and further stirred for 1 hour. The reaction mixture was treated with dry ice slowly and stirred for 30 min. It was then quenched with a saturated solution of ammonium chloride and extracted with ethylacetate (2×50 mL). The combined organic layers were dried over sodium sulfate, concentrated under reduced pressure to afford crude solid material, and then triturated with hexane/diethyl ether to afford pure 1-(tert-butoxycarbonylamino)indane-5-carboxylic acid (3.8 g). ¹H-NMR (400 MHz, DMSO): 7.8 (1H, s), 7.3 (1H, m), 7.2 (1H, m), 5.00 (1H, m), 2.9 (1H, m), 2.8 (1H, m), 2.31 (1H, m), 1.8 (1H, m), 1.45 (s, 9H). LC-MS (methanol, ESI): m/z=276 (M−H, retention time=1.74).

### EXAMPLE 16

### Preparation of tert-butyl N-[5-(trimethylsilylmethylcarbamoyl)indan-1-yl]carbamate

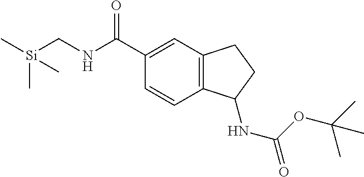 [SMILES: CC(C)(C)OC(=O)NC1CCc2cc(C(=O)NC[Si](C)(C)C)ccc21]

A solution of 1-(tert-butoxycarbonylamino)indane-5-carboxylic acid (2.1 g, 7.6 mmol) in dichloromethane (20 mL), was treated trimethylsilyl methylamine (0.88 g, 8.3 mmol), EDCI (1.4 g, 9.1 mmol) and a catalytic amount of DMAP (0.09 g, 0.76 mmol), and stirred for 20 hours. The reaction mixture was then treated with water (10 mL) and extracted with dichloromethane (2×25 mL). The combined organic layers were dried over sodium sulfate, concentrated, and purified by column chromatograph (hexane/ethylacetate as eluent) to afford tert-butyl N-[5-(trimethylsilylmethylcarbamoyl)indan-1-yl]carbamate (1.9 g). ¹H-NMR (400 MHz, CDCl3): 7.6 (1H, s), 7.53 (1H, d), 7.33 (1H, d), 6.00 (1H, m), 5.2 (1H, m), 4.8 (1H, m), 3.0 (1H, m), 2.9 (2H, d), 2.8 (1H, m), 2.6 (1H, m), 1.8 (1H, m), 1.45 (s, 9H), 0.2 (s, 9H). LC-MS (methanol, ESI): m/z=363 (M+H, retention time=2.05).

### EXAMPLE 17

### Preparation of tert-butyl N-[5-(trimethylsilylmethylcarbamothioyl)indan-1-yl]carbamate

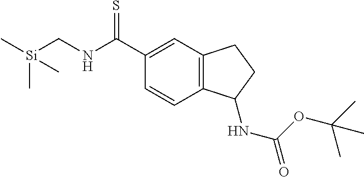 [SMILES: CC(C)(C)OC(=O)NC1CCc2cc(C(=S)NC[Si](C)(C)C)ccc21]

A solution of tert-butyl N-[5-(trimethylsilylmethylcarbamoyl)indan-1-yl]carbamate (2.2 g, 6.1 mmol) in toluene (25 mL), was treated with lawessons reagent (2.8 g, 6.7 mmol), and heated at 120° C. for 2 hours. The toluene was evaporated and the residue was treated with water (25 mL) and extracted with ethylacetate (25 mL). The combined organic layers were dried over sodium sulfate, concentrated, and purified by column chromatography (hexane/ethylacetate as eluent) to afford tert-butyl N-[5-(trimethylsilylmethylcarbamothioyl)indan-1-yl]carbamate (1.8 g). ¹H-NMR (400 MHz, CDCl3): 7.6 (1H, s), 7.53 (1H, brs), 7.45 (1H, d), 7.30 (1H, d), 5.2 (1H, brs), 4.7 (1H, brs), 3.5 (2H, d), 3.00 (1H, m), 2.8 (1H, m), 2.6 (1H, m), 1.8 (1H, m), 1.45 (s, 9H) 0.2 (s, 9H). LC-MS (methanol, ESI): m/z=379 (M+H, retention time=2.27).

### EXAMPLE 18

### Preparation of tert-butyl N-[5-[3-(3,5-dichlorophenyl)-3-(trifluoromethyl)-2,4-dihydropyrrol-5-yl]indan-1-yl]carbamate

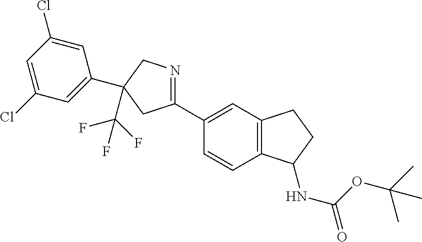 [SMILES: CC(C)(C)OC(=O)NC1CCc2cc(C3=NCC(c4cc(Cl)cc(Cl)c4)(C(F)(F)F)C3)ccc21]

A solution of tert-butyl N-[5-(trimethylsilylmethylcarbamothioyl)indan-1-yl]carbamate (1.2 g, 3.2 mmol) in dimethyl formanide (10 mL) at 0° C., was treated with potassium carbonate (0.9 g, 6.3 mmol) and methyl iodide (4.5 g, 32 mmol) added in three portions, and stirred for 3 hours. To the reaction mixture water (10 mL) was added, followed by extraction with ethylacetate (2×25 mL). The combined organic layers were dried over sodium sulfate, concentrated to afford crude material. The crude material was diluted with tetrahydrofuran (20 mL), cooled at 0° C., treated with 1,3-dichloro-5-[1-(trifluoromethyl)vinyl]benzene and TBAB, and stirred for 20 hours. Tetrahydrofuran was removed under reduced pressure, diluted with water (25 mL), and extracted with ethylacetate (2×25 mL). The combined organic layers were dried over sodium sulfate, concentrated and purified by column chromatography (hexane/ethyl acetate) gave tert-butyl N-[5-[3-(3,5-dichlorophenyl)-3-(trifluoromethyl)-2,4-dihydropyrrol-5-yl]indan-1-yl]carbamate (0.7 g). ¹H-NMR (400 MHz, CDCl3): 7.8 (2H, s), 7.7 (1H, m), 7.35 (2H, s), 7.25 (1H, d), 5.9 (1H, brs), 5.6 (1H, brs), 4.9 (1H, d), 4.4 (1H, d), 4.8 (1H, d), 3.40 (1H, d), 3.00 (1H, m), 2.90 (1H, m), 2.6 (1H, m), 2.00 (1H, m), 1.45 (s, 9H). LC-MS (methanol, ESI): m/z=513 (M+H, retention time=2.50).

### EXAMPLE 19

### General procedure for the preparation of N-[5-[3-(3,5-dichlorophenyl)-3-(trifluoromethyl)-2,4-dihydropyrrol-5-yl]indan-1-yl]amides: (a) R=Me, (b) R=Et, (c) R=cPr, (d)=CF₃

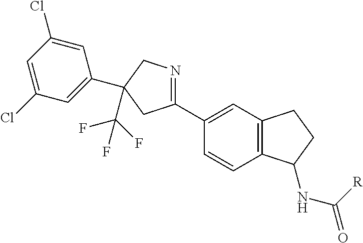

A solution of tert-butyl N-[5-[3-(3,5-dichlorophenyl)-3-(trifluoromethyl)-2,4-dihydropyrrol-5-yl]indan-1-yl]carbamate (1 mmol) in dichloromethane (5 mL), was treated with TFA (4 eq.), and stirred for 5 hours. The reaction mixture was concentrated under reduced pressure, diluted with dichloromethane (5 mL), and treated with triethylamine (5 eq.) and the appropriate carboxylic acid chloride (1.1 eq.) and stirred for 16 hours. The reaction mixture was concentrated and purified by column chromatography (hexane/ethyl acetate) to give:

(a) N-[5-[3-(3,5-dichlorophenyl)-3-(trifluoromethyl)-2,4-dihydropyrrol-5-yl]indan-1-yl]acetamide: 0.12 g, ¹H-NMR (400 MHz, CDCl3): 7.8 (1H, m), 7.7 (1H, m), 7.30 (1H, m), 7.20 (1H, m), 6.1 (1H, m), 5.5 (1H, m), 4.8 (1H, d), 4.4 (1H, d), 3.8 (1H, d), 3.45 (1H, d), 3.00 (1H, m), 2.90 (1H, m), 2.6 (1H, m), 2.00 (3H, s), 1.8 (1H, m). LC-MS (methanol, ESI): m/z=455 (M+H, retention time=2.11).

(b) N-[5-[3-(3,5-dichlorophenyl)-3-(trifluoromethyl)-2,4-dihydropyrrol-5-yl]indan-1-yl]propanamide: 0.12 g, ¹H-NMR (400 MHz, CDCl3): 7.9 (1H, m), 7.8 (1H, m), 7.40 (2H, m), 7.30 (2H, m), 5.70 (1H, m), 5.50 (1H, m), 4.90 (1H, d), 4.50 (1H, d), 3.90 (1H, d), 3.60 (1H, d), 3.00 (1H, m), 2.90 (1H, m), 2.6 (1H, m), 2.3 (2H, d), 1.8 (1H, m). 1.2 (3H, t) LC-MS (methanol, ESI): m/z=469 (M+H, retention time=2.17).

(c) N-[5-[3-(3,5-dichlorophenyl)-3-(trifluoromethyl)-2,4-dihydropyrrol-5-yl]indan-1-yl]cyclopropanecarboxamide: 70 mg, ¹H-NMR (400 MHz, CDCl3): 7.8 (1H, m), 7.77 (1H, m), 7.40 (2H, m), 7.30 (2H, m), 5.90 (1H, m), 5.50 (1H, m), 4.90 (1H, d), 4.45 (1H, d), 3.85 (1H, d), 3.50 (1H, d), 3.00 (1H, m), 2.90 (1H, m), 2.6 (1H, m), 1.8 (1H, m). 1.0 (2H, m), 0.8 (2H, m), LC-MS (methanol, ESI): m/z=481 (M+H, retention time=2.22)

(d) N-[5-[3-(3,5-dichlorophenyl)-3-(trifluoromethyl)-2,4-dihydropyrrol-5-yl]indan-1-yl]-2,2,2-trifluoro-acetamide: 120 mg, ¹H-NMR (400 MHz, CDCl3): 7.9 (1H, m), 7.8 (1H, m), 7.35 (2H, m), 7.25 (2H, m), 6.60 (1H, m), 5.50 (1H, m), 4.90 (1H, d), 4.50 (1H, d), 3.80 (1H, d), 3.50 (1H, d), 3.10 (1H, m), 3.00 (1H, m), 2.70 (1H, m), 2.00 (1H, m), LC-MS (methanol, ESI): m/z=509 (M+H, retention time=2.37)

### EXAMPLE 20

### Preparation of 5-[3-[3,5-bis(trifluoromethyl)phenyl]-3-(trifluoromethyl)pyrrolidin-1-yl]indan-1-amine

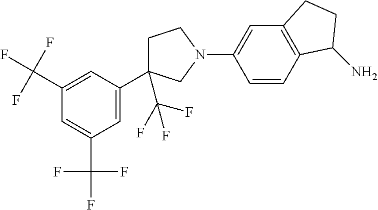 [SMILES: NC1CCc2cc(N3CCC(c4cc(C(F)(F)F)cc(C(F)(F)F)c4)(C(F)(F)F)C3)ccc21]

To a solution of 3-[3,5-bis(trifluoromethyl)phenyl]-3-(trifluoromethyl)pyrrolidine (0.260 g, prepared as described in WO 2008/128711) and tert-butyl N-(5-bromoindan-1-yl)carbamate (0.231 g) in toluene (5.2 mL) stirred under argon were added tris(dibenzylideneacetone)dipalladium(0) (13.6 mg), 4,5-bis(diphenylphosphino)-9,9-dimethylxanthene (22.1 mg) and sodium tert-butoxide (147 mg). The mixture was heated in the microwave at 130° C. for 15 min. The reaction was then diluted with ethylacetate and water then brine and then the mixture was extracted with ethyl acetate. The organic layers were combined and dried over magnesium sulphate, filtered then concentrated under reduced pressure to give a brown oil which was purified by chromatography on column (cyclohexane/EtOAc as solvent) to afford the desired product as a white foam (245 mg). 1H NMR (CDCl3, 400 MHz): d=7.92 (s, 1H), 7.86 (s, 2H), 7.27 (m, 1H), 6.47-6.58 (m, 2H), 4.35 (m, 1H), 4.05-4.23 (m, 1H), 3.85 (m, 1H), 3.60 (m, 1H), 3.51 (m, 1H), 2.90-3.03 (m, 2H), 2.71-2.86 (m, 1H), 2.55-2.66 (m, 1H), 2.50 (dt, J=7.2, 4.3 Hz, 1H), 1.83-2.13 (m, 2H), 1.72 ppm (dd, J=12.5, 8.1 Hz, 1H)

### EXAMPLE 21

### Preparation of N-[5-[3-[3,5-bis(trifluoromethyl)phenyl]-3-(trifluoromethyl)pyrrolidin-1-yl]indan-1-yl]propanamide

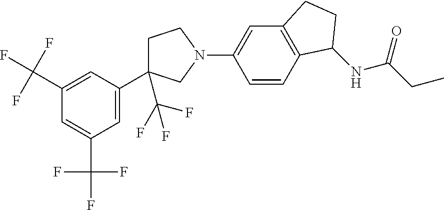 [SMILES: CCC(=O)NC1CCc2cc(N3CCC(c4cc(C(F)(F)F)cc(C(F)(F)F)c4)(C(F)(F)F)C3)ccc21]

To a solution of 5-[3-[3,5-bis(trifluoromethyl)phenyl]-3-(trifluoromethyl)pyrrolidin-1-yl]indan-1-amine (100 mg), in dichloromethane (3.0 mL) and triethylamine (0.088 mL) stirred at room temperature, was added propanoyl chloride (0.027 mL) and the solution was stirred for 3 hours. The reaction was then diluted with ethylacetate and water and then the mixture was extracted with ethyl acetate. The organic layers were combined and dried over magnesium sulphate, filtered then concentrated under reduced pressure to give a yellow solid which was purified by chromatography on column (cyclohexane/EtOAc as solvent) to afford the desired product as a beige solid (87 mg). 1H NMR (CDCl3, 400 MHz): d=7.92 (s, 1H), 7.86 (s, 2H), 7.21 (d, J=8.1 Hz, 1H), 6.45-6.58 (m, 2H), 5.56 (d, J=8.1 Hz, 1H), 5.37-5.49 (m, 1H), 4.16 (d, J=9.2 Hz, 1H), 3.85 (d, J=10.6 Hz, 1H), 3.46-3.60 (m, 2H), 2.91-3.04 (m, 2H), 2.78-2.91 (m, 1H), 2.52-2.70 (m, 2H), 2.24 (q, J=7.7 Hz, 2H), 1.76-1.87 (m, 1H), 1.13-1.24 ppm (m, 3H)

### EXAMPLE 22

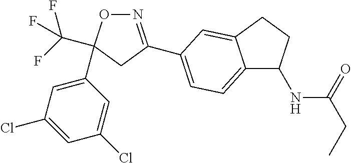 [SMILES: CCC(=O)NC1CCc2cc(C3=NOC(c4cc(Cl)cc(Cl)c4)(C(F)(F)F)C3)ccc21]

The synthesis was as described in WO2009/112275.

Melting point: 103-105° C.

LC-MS: retention time=1.95, (M⁺H)⁺=471 (measured) (Method B)

### EXAMPLE 23

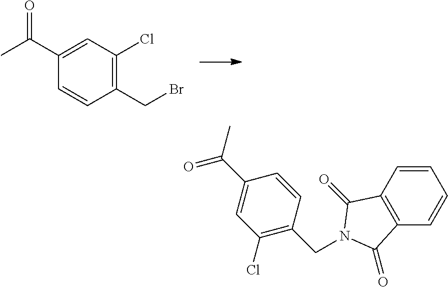 [SMILES: CC(=O)c1ccc(CBr)c(Cl)c1 ; CC(=O)c1ccc(CN2C(=O)c3ccccc3C2=O)c(Cl)c1]

To a solution of 1-[4-(bromomethyl)-3-chloro-phenyl]ethanone (1 g, 4.04 mmol) in N,N-dimethylformamide was added (1,3-dioxoisoindolin-2-yl) potassium (823 mg, 4.44 mmol). The reaction mixture was refluxed for 5-6 hours, while being monitored by Thin Layer Chromatography (TLC). The reaction mixture was then diluted with water (20 mL), extracted with ethylacetate (3×30 mL). The combined organic layers were dried over sodium sulphate then concentrated under reduced pressure. The residue obtained was purified by column chromatography to afford the desired product 2-[(4-acetyl-2-chloro-phenyl)methyl]isoindoline-1,3-dione (870 mg)

ESI-MS: m/z=313.9 [M+H]⁺.

¹H NMR (400 MHz, CDCl₃): δ 7.85-7.90 (m, 1H), 7.80-7.85 (m, 1H), 7.68-7.71 (m, 4H), 7.21-7.23 (m, 1H), 4.96 (s, 2H), 2.50 (s, 3H)

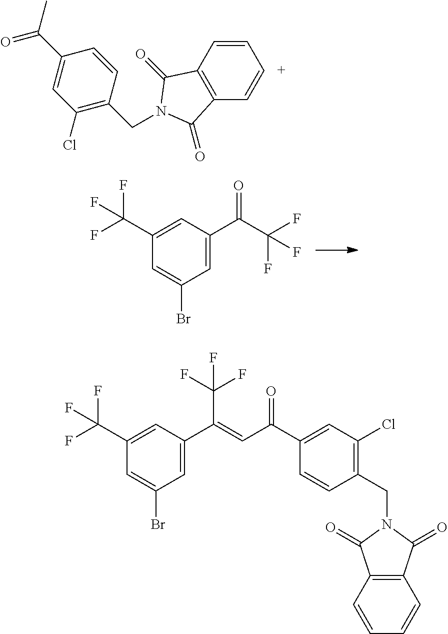 [SMILES: CC(=O)c1ccc(CN2C(=O)c3ccccc3C2=O)c(Cl)c1 ; O=C(c1cc(Br)cc(C(F)(F)F)c1)C(F)(F)F ; O=C(/C=C(/c1cc(Br)cc(C(F)(F)F)c1)C(F)(F)F)c1ccc(CN2C(=O)c3ccccc3C2=O)c(Cl)c1]

To a solution of 2-[(4-acetyl-2-chloro-phenyl)methyl]isoindoline-1,3-dione (670 mg, 2.13 mmol) and 1-[3-bromo-5-(trifluoromethyl)phenyl]-2,2,2-trifluoro-ethanone (685 mg, 2.13 mmol) in acetonitrile (10 mL) was added potassium carbonate (150 mg, 1.06 mmol). The reaction mixture was refluxed at 120° C. overnight. It was then diluted with water (10 mL) and extracted with ethylacetate (3×30 mL). The combined organic layers were dried over sodium sulphate then concentrated under reduced pressure. The residue obtained was purified by column chromatography to afford the desired product 2-[[4-[(Z)-3-[3-bromo-5-(trifluoromethyl)phenyl]-4,4,4-trifluoro-but-2-enoyl]-2-chloro-phenyl]methyl]isoindoline-1,3-dione (550 mg)

ESI-MS: m/z=614.76 [M−H]⁺.

¹H NMR (400 MHz, CDCl₃): δ 7.87-7.93 (m, 3H), 7.73-7.83 (m, 4H), 7.52-7.65 (m, 2H), 7.38-7.42 (m, 1H), 7.27-7.31 (m, 1H), 5.00-5.02 (d, 2H)

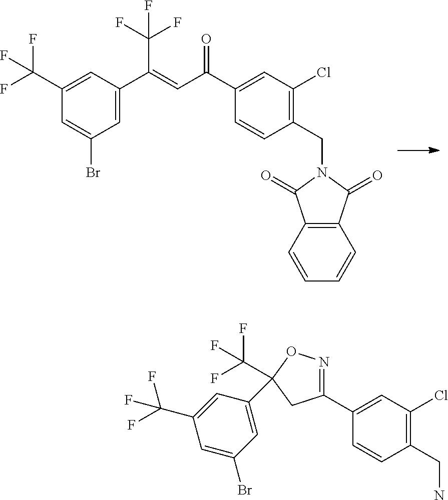 [SMILES: O=C(/C=C(/c1cc(Br)cc(C(F)(F)F)c1)C(F)(F)F)c1ccc(CN2C(=O)c3ccccc3C2=O)c(Cl)c1 ; NCc1ccc(C2=NOC(c3cc(Br)cc(C(F)(F)F)c3)(C(F)(F)F)C2)cc1Cl]

To a solution of 2-[[4-[(Z)-3-[3-bromo-5-(trifluoromethyl)phenyl]-4,4,4-trifluoro-but-2-enoyl]-2-chloro-phenyl]methyl]isoindoline-1,3-dione (500 mg, 0.813 mmol) in isopropyl alcohol (5 mL) was added a solution of sodium hydroxide (98 mg, 2.44 mmol) in water (2 mL) followed by 50% hydroxylamine solution (322 mg, 4.88 mmol). The reaction mixture was refluxed for 6-7 hours, while being monitored by TLC. The reaction mixture was concentrated under reduced pressure, diluted with water (10 mL) and extracted with ethylacetate (3×30 mL). The combined organic layers were dried over sodium sulphate then concentrated under reduced pressure. The residue obtained was purified by column chromatography to afford the desired product [4-[5-[3-bromo-5-(trifluoromethyl)phenyl]-5-(trifluoromethyl)-4H-isoxazol-3-yl]-2-chloro-phenyl]methanamine (170 mg) ESI-MS: m/z=500.84 [M+H]⁺.

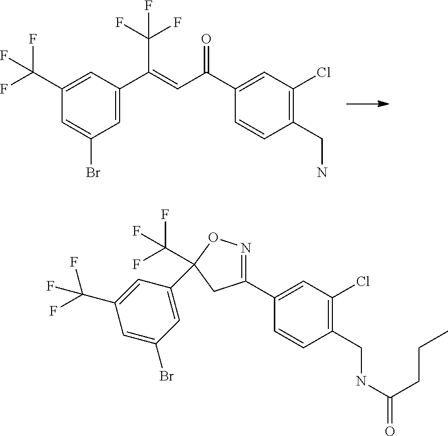 [SMILES: NCc1ccc(C(=O)/C=C(/c2cc(Br)cc(C(F)(F)F)c2)C(F)(F)F)cc1Cl ; CCCC(=O)NCc1ccc(C2=NOC(c3cc(Br)cc(C(F)(F)F)c3)(C(F)(F)F)C2)cc1Cl]

To a solution of [4-[5-[3-bromo-5-(trifluoromethyl)phenyl]-5-(trifluoromethyl)-4H-isoxazol-3-yl]-2-chloro-phenyl]methanamine (100 mg, 0.2 mmol) in dichloromethane (4 mL) at 0° C. was added triethylamine (0.05 mL, 0.3 mmol), followed by butyryl chloride (32 mg, 0.3 mmol). The reaction mixture was stirred at room temperature for 1 hour. It was then diluted with water (5 mL) and extracted with dichloromethane (3×20 mL). The combined organic layers were dried over sodium sulphate then concentrated under reduced pressure. The residue obtained was purified by column chromatography to afford the desired product N-[[4-[5-[3-bromo-5-(trifluoromethyl)phenyl]-5-(trifluoromethyl)-4H-isoxazol-3-yl]-2-chloro-phenyl]methyl]butanamide (50 mg)

ESI-MS: m/z=570.88 [M+H]⁺.

¹H NMR (400 MHz, CDCl₃): δ 7.88 (m, 1H), 7.77 (m, 1H), 7.72 (m, 1H), 7.62-7.63 (m, 1H), 7.38-7.46 (m, 2H), 5.89 (br s, 1H), 4.46-4.48 (d, 2H), 4.01-4.05 (d, 1H), 3.60-3.64 (d, 1H), 2.12-2.15 (m, 2H), 1.57-1.65 (m, 2H), 0.85-0.89 (m, 3H)

### BIOLOGICAL EXAMPLES

TABLE A

| | |
|---|---|
| | (Ia) |
| 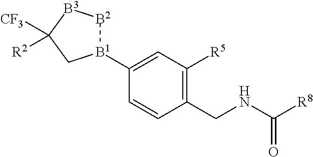 | |

| | B1—B2—B3 | R2 | R5 | R8 |
|---|---|---|---|---|
| A1 | C═N—O | 3,5-dichlorophenyl | chloro | methylsulfanyl- |
| | | | | methyl |
| A2 | C═N—O | 3,5-dichlorophenyl | chloro | isopropyl |
| A3 | C═N—O | 3,5-dichlorophenyl | chloro | ethyl |
| A4 | N—CH₂—CH₂ | 3,5-dichlorophenyl | methyl | methyl |
| A5 | C═N—CH₂ | 3,5-dichlorophenyl | bromo | methyl |
| A6 | C═N—CH₂ | 3,5-dichlorophenyl | bromo | ethyl |
| A7 | C═N—CH₂ | 3,5-dichlorophenyl | bromo | cyclopropyl |
| A8 | C═N—O | 3,5-dichlorophenyl | methyl | methyl |
| A9 | C═N—O | 3,5-dichlorophenyl | methyl | isopropyl |
| A10 | C═N—O | 3,5-dichlorophenyl | methyl | methylsulfanyl- |
| | | | | methyl |
| A11 | C═N—O | 3,5-dichlorophenyl | methyl | tetrahydro- |
| | | | | furan-2-yl |
| A12 | C═N—O | 3,5-dichlorophenyl | hydrogen | isopropyl |
| A13 | C═N—O | 3,5-dichlorophenyl | hydrogen | methylsulfanyl- |
| | | | | methyl |
| A14 | C═N—O | 3,5-dichlorophenyl | chloro | 1-bromo- |
| | | | | propan-1-yl |
| A15 | C═N—O | 3,5-dichlorophenyl | chloro | tetrahydro- |
| | | | | furan-2-yl |
| A16 | C═N—O | 3,5-dichlorophenyl | hydrogen | ethyl |
| A17 | C═N—O | 3,5-dichlorophenyl | hydrogen | 1-bromo- |
| | | | | propan-1-yl |
| A18 | C═N—O | 3,5-dichlorophenyl | hydrogen | tetrahydro- |
| | | | | furan-2-yl |
| A19 | C═N—O | 3,5-dichlorophenyl | chloro | methylsulfonyl- |
| | | | | methyl |
| A20 | C═N—O | 3,5-dichlorophenyl | chloro | methylsulfinyl- |
| | | | | methyl |
| A21 | C═N—O | 3-bromo-5- | chloro | n-propyl |
| | | trifluorophenyl | | |

Tables A provides compounds of formula Ia wherein B¹, B², B³, R², R⁵, and R⁸ are as described below

TABLE B

| | |
|---|---|
| | (Ib*) |
| 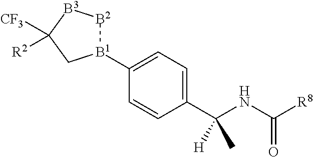 | |

| | B1—B2—B3 | R2 | R8 |
|---|---|---|---|
| B1 | N—CH₂—CH₂ | 3,5-dichlorophenyl | cyclopropyl |
| B2 | C═N—O | 3,5-dichlorophenyl | —CH₃ |
| B3 | C═N—CH₂ | 3,4,5-trichlorophenyl | cyclopropyl |
| B4 | N—CH₂—CH₂ | 3,5-di- | cyclopropyl |
| | | trifluoromethylphenyl | |

Tables B provides compounds of formula Ib* wherein B¹, B², B³, R² and R⁸ are as described below

TABLE C

| | |
|---|---|
| | (Ic) |
| 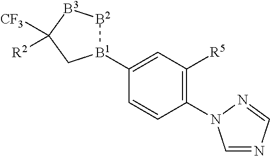 | |

| | B1—B2—B3 | R2 | R5 |
|---|---|---|---|
| C1 | N—CH₂—CH₂ | 3,5-dichlorophenyl | cyano |
| C2 | C═N—CH₂ | 3,4,5-trichlorophenyl | cyano |
| C3 | C═N—O | 3,5-dichloro-4-fluorophenyl | cyano |
| C4 | C═N—O | 3,5-dichlorophenyl | cyano |
| C5 | C═N—O | 3,5-dichlorophenyl | methyl |
| C6 | C═N—O | 3,4,5-trichlorophenyl | cyano |
| C7 | C═N—O | 3,5-dichlorophenyl | cyano |

Tables C provides compounds of formula Ic wherein B¹, B², B³, R² and R⁵ are as described below

TABLE D

| | |
|---|---|
| | (Id) |
| 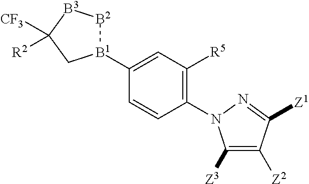 | |

| | B1—B2—B3 | R2 | R5 | Z1 | Z2 | Z3 |
|---|---|---|---|---|---|---|
| D1 | C═N—O | 3,5-dichloro-4- | cyano | H | cyano | H |
| | | fluorophenyl | | | | |
| D2 | C═N—O | 3,5-dichloro-4- | cyano | H | F | H |
| | | fluorophenyl | | | | |
| D3 | C═N—O | 3,5-dichloro- | cyano | H | H | H |
| | | phenyl | | | | |
| D4 | C═N—O | 3,5-dichloro- | amino | H | H | H |
| | | phenyl | | | | |
| D5 | C═N—O | 3,5-dichloro- | methyl | H | H | H |
| | | phenyl | | | | |
| D6 | C═N—O | 3,5-dichloro-4- | cyano | H | chloro | H |
| | | fluorophenyl | | | | |
| D7 | C═N—O | 3,5-dichloro-4- | cyano | H | bromo | H |
| | | fluorophenyl | | | | |
| D8 | C═N—O | 3,5-dichloro-4- | cyano | H | iodo | H |
| | | fluorophenyl | | | | |
| D9 | C═N—O | 3,5-dichloro-4- | cyano | H | nitro | H |
| | | fluorophenyl | | | | |
| D10 | C═N—O | 3,5-dichloro-4- | cyano | CF3 | H | CF3 |
| | | fluorophenyl | | | | |
| D11 | C═N—O | 3,5-dichloro-4- | cyano | CF3 | H | H |
| | | fluorophenyl | | | | |

Tables D provides compounds of formula Id wherein B¹, B², B³, R², R⁵, Z¹, Z² and Z³ are as described below

TABLE E

| | |
|---|---|
| | (Ie) |
| 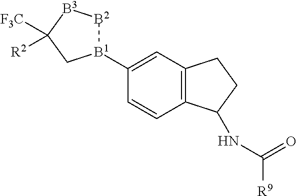 | |

| | B¹—B²—B³ | R² | R⁹ |
|---|---|---|---|
| E1 | C═N—O | 3,5-dichlorophenyl | ethyl |
| E2 | C═N—CH₂ | 3,5-dichlorophenyl | methyl |
| E3 | C═N—CH₂ | 3,5-dichlorophenyl | ethyl |
| E4 | C═N—CH₂ | 3,5-dichlorophenyl | cyclopropyl |
| E5 | C═N—CH₂ | 3,5-dichlorophenyl | trifluoromethyl |
| E6 | N—CH₂—CH₂ | 3,5-bis(trifluoromethyl)phenyl | Ethyl |
| E7 | C═N—O | 3,5-dichlorophenyl | 2,2,2-trifluoroethyl |
| E8 | C═N—O | 3,5-dichlorophenyl | 1-chloropropan-1-yl |

Tables E provides compounds of formula Ie wherein B¹, B², B³, R², and R⁹ are as described below

Anthonomus grandis (Cotton Boll Weevil)

Cotton plants are treated (100 mL spray volume) and 10 adult weevils are added to each plant (2 replicates per concentration). 5 days after infestation the mortality is assessed. The following compounds were tested and gave at least 80% control at 50 ppm: B3, B4, C2, C3, C4, E1, E7

### COMPARATIVE EXAMPLE

Compounds are tested according to the above method. The results show that the compounds of the invention are significantly more active against Anthonomus grandis (Cotton boll weevil) than structurally similar compounds, particularly at low rates of application.

COMPARATIVE TABLE 1

| 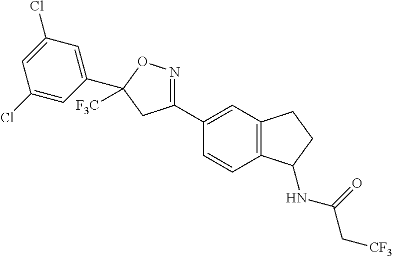 | | Compound of the invention | |
|---|---|---|---|
| 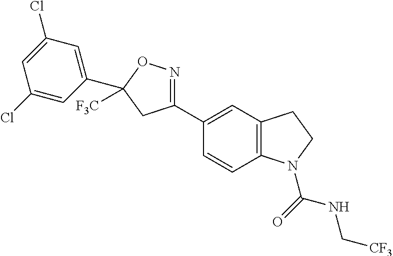 [SMILES: O=C(NCC(F)(F)F)N1CCc2cc(C3=NOC(c4cc(Cl)cc(Cl)c4)(C(F)(F)F)C3)ccc21] | | Reference compound | |
| | | Application rate/ | Control/ |
| Compound | Test | ppm | % |
| Compound of the invention | Anthonomus grandis (Cotton | 50 | 100 |
| | boll weevil) | 12.5 | 100 |
| Reference compound | Anthonomus grandis (Cotton | 50 | 10 |
| | boll weevil) | 12.5 | 0 |

The compound of the invention and reference compound are compounds 3-6 and 3-643 respectively from WO 2009/112275.

COMPARATIVE TABLE 2

| 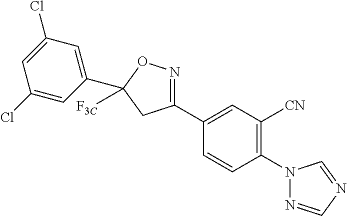 [SMILES: N#Cc1cc(C2=NOC(c3cc(Cl)cc(Cl)c3)(C(F)(F)F)C2)ccc1-n1cncn1] | | Compound of the invention | |
|---|---|---|---|
| 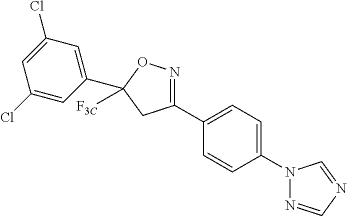 [SMILES: FC(F)(F)C1(c2cc(Cl)cc(Cl)c2)CC(c2ccc(-n3cncn3)cc2)=NO1] | | Reference compound | |
| | | Application rate/ | Control/ |
| Compound | Test | ppm | % |
| Compound of the invention | Anthonomus grandis (Cotton | 50 | 90 |
| | boll weevil) | 12.5 | 10 |
| Reference compound | Anthonomus grandis (Cotton | 50 | 10 |
| | boll weevil) | 12.5 | 5 |

The compound of the invention and reference compound are compounds 42 and 9 respectively from WO 2007/075459.

1. A method comprising applying to a crop of cotton plants, the locus thereof, or propagation material thereof, a compound of formula IC 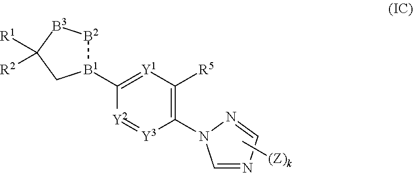
wherein -B¹-B²-B³- is —C═N—O—, —C═N—CH₂—, or —N—CH₂—CH₂—;
R¹ is trifluoromethyl, difluoromethyl or chlorodifluoromethyl;
R² is group X 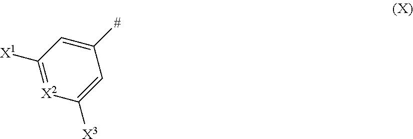
X² is C—X⁶ or nitrogen;
X¹, X³ and X⁶ are independently hydrogen, halogen or trihalomethyl, wherein at least one of X¹, X³ and X⁶ is not hydrogen;
Y¹ is C—R⁶, CH or nitrogen;
Y² and Y³ are independently CH or nitrogen;
wherein no more than two of Y¹, Y² and Y³ are nitrogen and wherein Y² and Y³ are not both nitrogen;
R⁵ is halogen, cyano, nitro, NH₂, C₁-C₄alkyl, C₁-C₄haloalkyl, C₃-C₅cycloalkyl, C₃-C₅halocycloalkyl, C₁-C₂alkoxy, or C₁-C₂haloalkoxy;
R⁶ when present together with R⁵ forms a —CH═CH—CH═CH— bridge;
each Z is independently halogen, C₁-C₁₂alkyl or C₁-C₁₂alkyl substituted by one to five R¹², nitro, C₁-C₁₂alkoxy or C₁-C₁₂alkoxy substituted by one to five R¹², cyano, C₁-C₁₂alkylsulfinyl, C₁-C₁₂alkylsulfonyl, C₁-C₁₂haloalkylsulfinyl, C₁-C₁₂haloalkylsulfonyl, hydroxyl or thiol;
each R¹² is halogen, cyano, nitro, hydroxy, C₁-C₈alkoxy-, C₁-C₈haloalkoxy-, mercapto, C₁-C₈alkylthio-, or C₁-C₈haloalkylthio; and
k is 0, 1, 2 or 3.

2. A method according to claim 1, wherein
R⁵ is cyano;
k is 0 or 1; and
Z is cyano or trifluoromethyl.

3. A method according to claim 1 or claim 2, wherein
R¹ is CF₃;
-B¹-B²-B³- is —C═N—O— or —C═N—CH₂—;
Y¹, Y² and Y³ are CH; and
k is 0.

4. A method comprising applying to a crop of cotton plants, the locus thereof, or propagation material thereof, a compound of formula ID 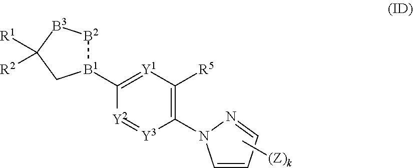
wherein -B¹-B²-B³- is —C═N—O—, —C═N—CH₂—, or —N—CH₂—CH₂—;
R¹ is trifluoromethyl, difluoromethyl or chlorodifluoromethyl;
R² is group X 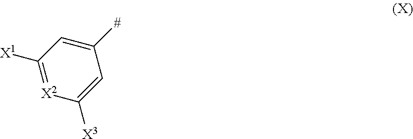
X² is C—X⁶ or nitrogen;
X¹, X³ and X⁶ are independently hydrogen, halogen or trihalomethyl, wherein at least one of X¹, X³ and X⁶ is not hydrogen;
Y¹ is C—R⁶, CH or nitrogen;
Y² and Y³ are independently CH or nitrogen;
wherein no more than two of Y¹, Y² and Y³ are nitrogen and wherein Y² and Y³ are not both nitrogen;
R⁵ is halogen, cyano, nitro, NH₂, C₁-C₄alkyl, C₁-C₄haloalkyl, C₃-C₅cycloalkyl, C₃-C₅halocycloalkyl, C₁-C₂alkoxy, or C₁-C₂haloalkoxy;
R⁶ when present together with R⁵ forms a —CH═CH—CH═CH— bridge;
each Z is independently halogen, C₁-C₁₂alkyl or C₁-C₁₂alkyl substituted by one to five R¹², nitro, C₁-C₁₂alkoxy or C₁-C₁₂alkoxy substituted by one to five R¹², cyano, C₁-C₁₂alkylsulfinyl, C₁-C₁₂alkylsulfonyl, C₁-C₁₂haloalkylsulfinyl, C₁-C₁₂haloalkylsulfonyl, hydroxyl or thiol;
each R¹² is halogen, cyano, nitro, hydroxy, C₁-C₈alkoxy-, C₁-C₈haloalkoxy-, mercapto, C₁-C₈alkylthio-, or C₁-C₈haloalkylthio; and
k is 0, 1, 2 or 3.

5. A method according to claim 4, wherein
wherein
R⁵ is cyano;
k is 0 or 1; and
Z is cyano or trifluoromethyl;
and when k is 1, Z is attached to the 4 position of the imidazole moiety.

6. A method according to claim 4, wherein
R¹ is CF₃;
-B¹-B²-B³- is —C═N—O— or —C═N—CH₂—;
Y¹, Y² and Y³ are CH;
k is 1; and
Z is cyano or trifluoromethyl;
and Z is attached to the 4 position of the imidazole moiety.

7. A method comprising applying to a crop of cotton plants, the locus thereof, or propagation material thereof, a compound of formula IE 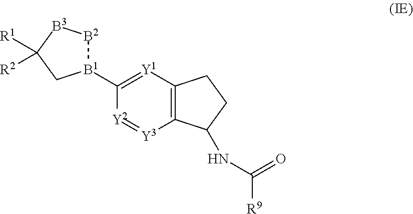
wherein -B¹-B²-B³- is —C═N—O—, —C═N—CH₂—, or —N—CH₂—CH₂—;
R¹ is trifluoromethyl, difluoromethyl or chlorodifluoromethyl;
R² is group X 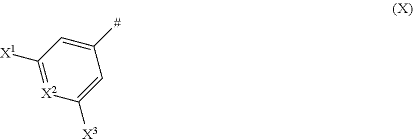
X² is C—Xe or nitrogen;
X¹, X³ and X⁶ are independently hydrogen, halogen or trihalomethyl, wherein at least one of X¹, X³ and X⁶ is not hydrogen;
Y¹ is CH or nitrogen;
Y² and Y³ are independently CH or nitrogen;
wherein no more than two of Y¹, Y² and Y³ are nitrogen and wherein Y² and Y³ are not both nitrogen; and
R⁹ is C₁-C₄alkyl, C₁-C₄haloalkyl, C₁-C₄alkyl-O—CH₂—, C₁-C₄haloalkyl-O—CH₂—, C₃-C₆cycloalkyl, C₃-C₆cycloalkyl-CH₂—, C₁-C₄alkyl-S—CH₂—, C₁-C₄alkyl-S(O)—CH₂—, or C₁-C₄alkyl-S(O₂)—CH₂.

8. A method according to claim 7, wherein R⁹ is methyl, ethyl, propyl, CF₃CH₂— or cyclopropyl.

9. A method according to claim 7 or claim 8, wherein
R¹ is CF₃;
-B¹-B²-B³- is —C═N—O— or —C═N—CH₂—; and
Y¹, Y² and Y³ are CH.

10. A method comprising applying to a crop of cotton plants, the locus thereof, or propagation material thereof, a compound of formula IA 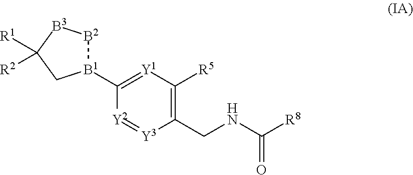
wherein -B¹-B²-B³- is —C═N—O—, —C═N—CH₂—, or —N—CH₂—CH₂—;
R¹ is trifluoromethyl, difluoromethyl or chlorodifluoromethyl;
R² is group X 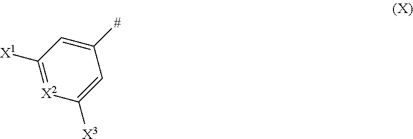
X² is C—X⁶ or nitrogen;
X¹, X³ and X⁶ are independently hydrogen, halogen or trihalomethyl, wherein at least one of X¹, X³ and X⁶ is not hydrogen;
Y¹ is C—R⁶, CH or nitrogen;
Y² and Y³ are independently CH or nitrogen;
wherein no more than two of Y¹, Y² and Y³ are nitrogen and wherein Y² and Y³ are not both nitrogen;
R⁵ is hydrogen, halogen, cyano, nitro, NH₂, C₁-C₄alkyl, C₁-C₄haloalkyl, C₃-C₅cycloalkyl, C₃-C₅halocycloalkyl, C₁-C₂alkoxy, or C₁-C₂haloalkoxy;
R⁶ when present together with R⁵ forms a —CH═CH—CH═CH— bridge; and
R⁸ is C₁-C₄alkyl, C₁-C₄haloalkyl, C₁-C₄alkoxy(C₁-C₄)alkyl, C₁-C₄alkylthio(C₁-C₄)alkyl, C₁-C₄alkylsulfinyl(C₁-C₄)alkyl, C₁-C₄alkylsulfonyl(C₁-C₄)alkyl, C₃-C₆cycloalkyl, C₃-C₆cycloalkyl(C₁-C₄)alkyl-, or tetrahydrofuranyl.

11. A method according to claim 10, wherein
R⁵ is chloro, bromo, fluoro or methyl; and
R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—.

12. A method or use according to claim 10,
wherein
R¹ is CF₃;
-B¹-B²-B³- is —C═N—O— or —C═N—CH₂—;
Y¹, Y² and Y³ are CH; and
R⁵ is chloro or methyl.

13. A method according to claim 10, wherein
-B¹-B²-B³- is —C═N—O—, R¹ is trifluoromethyl, R² is 3-bromo-5-trifluoromethylphenyl, Y¹, Y² and Y³ are CH; R⁵ is chloro, and R⁸ is n-propyl.

14. A method comprising applying to a crop of cotton plants, the locus thereof, or propagation material thereof, a compound of formula IB 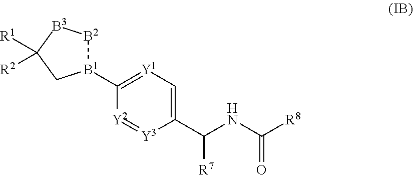
wherein -B¹-B²-B³- is —C═N—O—, —C═N—CH₂—, or —N—CH₂—CH₂—;
R¹ is trifluoromethyl, difluoromethyl or chlorodifluoromethyl;
R² is group X 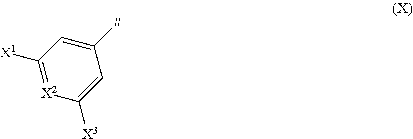
X² is C—X⁶ or nitrogen;
X¹, X³ and X⁶ are independently hydrogen, halogen or trihalomethyl, wherein at least one of X¹, X³ and X⁶ is not hydrogen;
Y¹ is CH or nitrogen;
Y² and Y³ are independently CH or nitrogen;
wherein no more than two of Y¹, Y² and Y³ are nitrogen and wherein Y² and Y³ are not both nitrogen;
R⁷ is C₁-C₄alkyl; and
R⁸ is C₁-C₄alkyl, C₁-C₄haloalkyl, C₁-C₄alkoxy(C₁-C₄)alkyl, C₁-C₄alkylthio(C₁-C₄)alkyl, C₁-C₄alkylsulfinyl(C₁-C₄)alkyl, C₁-C₄alkylsulfonyl(C₁-C₄)alkyl, C₃-C₆cycloalkyl, C₃-C₆cycloalkyl(C₁-C₄)alkyl-, or tetrahydrofuranyl.

15. A method according to claim 14, wherein
R⁷ is methyl; and
R⁸ is methyl, ethyl, n-propyl, isopropyl, CH₃—O—CH₂—, CH₃—S—CH₂—, CH₃—S(O)—CH₂—, CH₃—SO₂—CH₂—, cyclobutyl, cyclopropyl or cyclopropyl-CH₂—.

16. A method according to claim 14, wherein
R¹ is CF₃;
-B¹-B²-B³- is —C═N—O—, —C═N—CH₂— or —N—CH₂—CH₂—; and
Y¹, Y² and Y³ are CH.

17. A method according to claim 1, wherein R² is 3,5-dichlorophenyl-, 3,5-dichloro-4-fluorophenyl-, 3,4,5-trichlorophenyl- or 3,5-bis(trifluoromethyl)phenyl.

18. A method according to claim 1, wherein the method is a method of controlling and/or preventing infestation of insects the family Curculionidae in cotton comprising applying to a crop of cotton plants, the locus thereof, or propagation material thereof, a compound of formula I.

19. A method of controlling and/or preventing infestation of insects the family Curculionidae in a crop of useful plants comprising applying to a crop of useful plants, the locus thereof, or propagation material thereof, a compound of formula I as defined in claim 1.

20. (canceled)

21. A method according to claim 1, wherein said insects are chosen from a member of the genus Anthonomus and Anthonomus grandis

22. A method according to claim 1, wherein said insects are selected from the group consisting of Anthonomus corvulus, Anthonomus elutus, Anthonomus elongatus, Anthonomus eugenii, Anthonomus consors, Anthonomus haematopus, Anthonomus lecontei, Anthonomus molochinus, Anthonomus morticinus, Anthonomus musculus, Anthonomus nigrinus, Anthonomus phyllocola, Anthonomus pictus, Anthonomus pomorum, Anthonomus quadrigibbus, Anthonomus rectirostris, Anthonomus rubi, Anthonomus santacruzi, Anthonomus signatus, Anthonomus subfasciatus, and Anthonomus tenebrosus.

23. A method according to claim 1, wherein the compound of formula I is a mixture of the compound of formula I* and the compound of formula I** 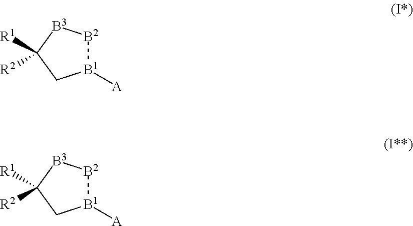
wherein A represents A1, A2, A3 A4 or A5 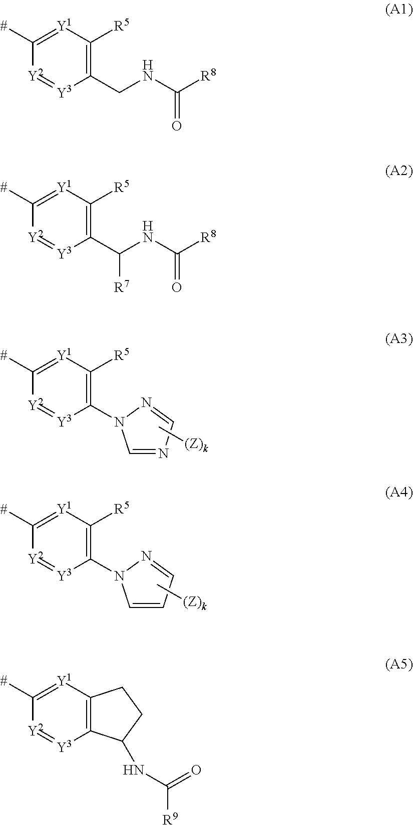
and wherein the remaining substituents are as defined in claim 1, providing that R⁵ is hydrogen when A represents A2, and wherein said mixture is enriched for the compound of formula I**.

24. A method according to claim 7, wherein the compound of formula IE is a mixture of compounds IE-a and IE-b 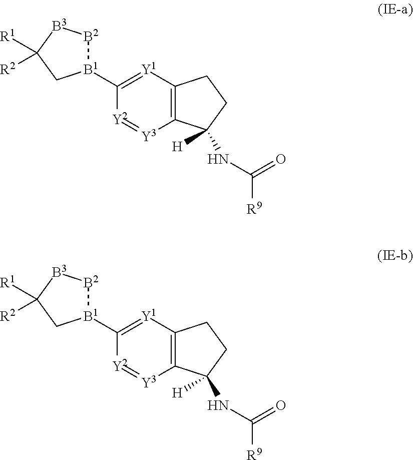
wherein the molar proportion of compound IE-a compared to the total amount of IE-a and IE-b is greater than 50%.

25. (canceled)

26. (canceled)
